SEQUENCE LISTING (62 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 3 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 4 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 70 aa, Homo sapiens
FVNQHLCGSHLVEALYLVCGERGFFYTPKTRREAEDLQVGQVELGGGPGAGSLQPLALEGSLQKRGIVEQ

SEQ ID NO: 6 - protein, 62 aa, Homo sapiens
SHLVEALYLVCGERGFFYTPKTRREAEDLQVGQVELGGGPGAGSLQPLALEGSLQKRGIVEQ

SEQ ID NO: 7 - protein, 30 aa, Homo sapiens
SHLVEALYLVCGERGFFYTPKTRREAEDLQ

SEQ ID NO: 8 - protein, 38 aa, Homo sapiens
FVNQHLCGSHLVEALYLVCGERGFFYTPKTRREAEDLQ

SEQ ID NO: 9 - protein, 15 aa, Homo sapiens
SHLVEALYLVCGERG

SEQ ID NO: 10 - protein, 27 aa, Homo sapiens
GGGPGAGSLQPLALEGSLQKRGIVEQC

SEQ ID NO: 11 - protein, 11 aa, Homo sapiens
LALEGSLQKRG

SEQ ID NO: 12 - protein, 18 aa, Homo sapiens
GSLQPLALEGSLQKRGIV

SEQ ID NO: 13 - protein, 20 aa, Homo sapiens
GGGPGAGSLQPLALEGSLQK

SEQ ID NO: 14 - protein, 28 aa, Homo sapiens
SHLVEALYLVCGERGFFYTPKTRREAED

SEQ ID NO: 15 - protein, 17 aa, Homo sapiens
QPLALEGSLQKRGIVEQ

SEQ ID NO: 16 - protein, 65 aa, Homo sapiens
AYQAEPNTCATAQGEGNIKKNRHPDFLPYDHARIKLKVESSPSRSDYINASPIIEHDPRMPAYIA

SEQ ID NO: 17 - protein, 35 aa, Homo sapiens
GPLSHTIADFWQMVWESGCTVIVMLTPLVEDGVKQ

SEQ ID NO: 18 - protein, 56 aa, Homo sapiens
GASLYHVYEVNLVSEHIWCEDFLVRSFYLKNVQTQETRTLTQFHFLSWPAEGTPAS

SEQ ID NO: 19 - protein, 39 aa, Homo sapiens
EHVRDQRPGLVRSKDQFEFALTAVAEEVNAILKALPQCG

SEQ ID NO: 20 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 21 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 22 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 23 - protein, 20 aa, Homo sapiens
KYLATASTMDHARHGFLPRH

SEQ ID NO: 24 - protein, 16 aa, Homo sapiens
ENPWHFFKNIVTPRTP

SEQ ID NO: 25 - protein, 19 aa, Homo sapiens
LSRFSWGAEGQRPGFGYGG

SEQ ID NO: 26 - protein, 25 aa, Homo sapiens
AQGTLSKIFKLGGRDSRSGSPMARR

SEQ ID NO: 27 - protein, 20 aa, Homo sapiens
GQFRVIGPRHPIRALVGDEV

SEQ ID NO: 28 - protein, 20 aa, Homo sapiens
MEVGWYRPPFSRWHLYRNGK

SEQ ID NO: 29 - protein, 16 aa, Homo sapiens
HCLGKWLGHPDKFVGI

SEQ ID NO: 30 - protein, 31 aa, Homo sapiens
KNATGMEVGWYRSPFSRVVHLYRNGKDQDAE

SEQ ID NO: 31 - protein, 17 aa, Homo sapiens
ENPVVHFFKNIVTPRTP

SEQ ID NO: 32 - protein, 21 aa, Homo sapiens
MEVGWYRPPFSRVVHLYRNGK

SEQ ID NO: 33 - protein, 17 aa, Homo sapiens
DENPVVHFFKNIVTPRT

SEQ ID NO: 34 - protein, 18 aa, Homo sapiens
DENPVVHFFKNIVTPRTP

SEQ ID NO: 35 - protein, 25 aa, Homo sapiens
DENPVVHFFKNIVTPRTPPPSQGKG

SEQ ID NO: 36 - protein, 20 aa, Homo sapiens
VHFFKNIVTPRTPPPSQGKG

SEQ ID NO: 37 - protein, 25 aa, Homo sapiens
ASDYKSAHKGLKGVDAQGTLSKIFK

SEQ ID NO: 38 - protein, 18 aa, Homo sapiens
GTEKLIETYFSKNYQDYE

SEQ ID NO: 39 - protein, 18 aa, Homo sapiens
GFYTTGAVRQIFGDYKTT

SEQ ID NO: 40 - protein, 22 aa, Homo sapiens
AVRQIFGDYKTTICGKGLSATV

SEQ ID NO: 41 - protein, 20 aa, Homo sapiens
NTWTTCQSIAFPSKTSASIG

SEQ ID NO: 42 - protein, 20 aa, Homo sapiens
SKTSASIGSLCADARMYGVL

SEQ ID NO: 43 - protein, 20 aa, Homo sapiens
PIRALVGDEVELPCRISPGK

SEQ ID NO: 44 - protein, 20 aa, Homo sapiens
ELPCRISPGKNATGMEVGWY

SEQ ID NO: 45 - protein, 23 aa, Homo sapiens
EYRGRTELLKDAIGEGKVTLRIR

SEQ ID NO: 46 - protein, 62 aa, Homo sapiens
GQFRVIGPRHPIRALVGDEVELPCRISPGKNATGMEVGWYRPPFSRVVHLYRNGKDQDGDQA

SEQ ID NO: 47 - protein, 63 aa, Homo sapiens
SHGRTQDENPVVHFFKNIVTPRTPPPSQGKGRGLSLSRFSWGAEGQRPGFGYGGRASDYKSCG

SEQ ID NO: 48 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 49 - protein, 529 aa, Homo sapiens [sequence omitted]

SEQ ID NO: 50 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 51 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 52 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 53 - protein, 1247 aa, Homo sapiens
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 54 - protein, 33 aa, Homo sapiens
LQLQPFPQPQLPYPQPQLPYPQPQLPYPQPQPF

SEQ ID NO: 55 - protein, 33 aa, Homo sapiens
LQLQPFPQPELPYPQPELPYPQPELPYPQPQPF

SEQ ID NO: 56 - protein, 18 aa, Homo sapiens
QQYPSGQGSFQPSQQNPQ

SEQ ID NO: 57 - protein, 12 aa, Homo sapiens
QPFPQPEQPFPW

SEQ ID NO: 58 - protein, 15 aa, Homo sapiens
ELQPFPQPELPYPQP

SEQ ID NO: 59 - protein, 18 aa, Homo sapiens
GCRGGGPQPQPFPSQQPY

SEQ ID NO: 60 - protein, 51 aa, Homo sapiens
GCRGGGPQPQPFPSQQPYLQLQPFPQPQLPYPQPQLPYPQPQLPYPQPQPF

SEQ ID NO: 61 - protein, 51 aa, Homo sapiens
GCRGGGPQPQPFPSQQPYLQLQPFPQPELPYPQPELPYPQPELPYPQPQPF

SEQ ID NO: 62 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2026/0151477 A1
(43) Pub. Date: Jun. 4, 2026

(54) COMPOSITIONS AND METHODS CONCERNING IMMUNE TOLERANCE

(71) Applicants: THE UNIVERSITY OF CHICAGO, Chicago, IL (US); ANOKION SA, Ecublens (CH)

(72) Inventors: Jeffrey A. HUBBELL, Chicago, IL (US); David Scott WILSON, Chicago, IL (US); Kym BRÜNGGEL, Chicago, IL (US); Kristen Marie LORENTZ, Ecublens (CH)

(73) Assignees: THE UNIVERSITY OF CHICAGO, Chicago, IL (US); Anokion SA, Ecublens (CH)

(21) Appl. No.: 19/276,090

(22) Filed: Jul. 22, 2025

Related U.S. Application Data
(63) Continuation of application No. 17/250,015, now Pat. No. 12,383,617.
(60) Provisional application No. 62/669,044, filed on May 9, 2018.

(51) Int. Cl. A61K 39/385; A61K 39/00; A61K 40/11; A61K 40/42; A61K 47/54; A61K 47/60; A61P 37/06; C07D 249/16; C08F 220/58; C08G 65/332; C08G 65/334; C08G 81/02

(52) U.S. Cl. CPC A61K 39/385; A61K 39/0008; A61K 39/001154; A61K 39/001158; A61K 40/11; A61K 40/42; A61K 47/549; A61K 47/60; A61P 37/06; C07D 249/16; C08F 220/58; C08G 65/332; C08G 65/3344; C08G 81/025; A61K 2039/6087; A61K 2039/6093; A61K 2239/38

(57) ABSTRACT

The present disclosure provides compositions comprising mannose-fused antigens to target mannose receptors. The compositions may be used to prevent immunity or reduce an immune response protein-based drugs that would otherwise elicit an immune response.

### CROSS-REFERENCE TO RELATED APPLICATIONS

This application is a continuation of U.S. patent application Ser. No. 17/250,015, filed Nov. 3, 2020, which is a national phase application under 35 U.S.C. § 371 of International Application No. PCT/US2019/031440, filed May 9, 2019, which claims priority to U.S. Provisional Patent Application No. 62/669,044, filed May 9, 2018, all of which are incorporated in their entirety by reference.

### SEQUENCE LISTING

The instant application contains a Sequence Listing which has been submitted in XML format and is hereby incorporated by reference in its entirety. Said XML copy, created on Mar. 14, 2025, is named ARCDP00659USCI-Sequence-Listing.xml and is 60,455 bytes in size.

### BACKGROUND OF THE INVENTION

### 1. Field

Embodiments of the present invention relate generally to the fields of organic chemistry, biochemistry, and immunology.

### 2. Background

Small molecules, e.g., non-peptide or nucleic acid-based molecules having a molecular weight of less than 500 Daltons, have historically dominated the FDA's new molecular entity (NME) approvals. Since the FDA's approval of insulin as the first drug produced by recombinant DNA technology in 1982, protein-based drug approvals have experienced an upward trend, as demonstrated by the almost yearly increase in FDA biologics license application (BLA) approvals in the last 23 years.

A primary difference between a small molecule drug and a protein-based drug lies in the ability of antigen presenting cells to take up proteins, including protein-based drugs, process them, and present them as peptides to the major histocompatibility complex (MHC) class I and II to the immune system. Protein-based drugs are expressed and purified to be free of any pathogen-associated molecular patterns (PAMPs) and danger associated molecules (DAMPs). Notwithstanding the anti-immunogenic design of protein-based drugs, a significant portion of patients who are treated with these drugs develop antibodies against the drugs (anti-drug antibodies).

These and other examples demonstrate that protein-based drug immunogenicity can revert the effect of therapy for certain patients or even render a new approach to treat a disease inefficient. The negative effects provided by anti-drug antibodies highlight the need to develop a platform that allows clinicians induce tolerance to foreign and partly foreign proteins.

Technologies to induce antigen-specific immunological tolerance are still in their infancy, but are needed to prevent immunity to many protein-based drugs, to reverse immunity to allergens, and to prevent and reverse immunity to autoimmune antigens.

### SUMMARY

A useful tolerogenic drug would consist of an antigen and a tolerance inducing component. The tolerance inducing component can be a chemical conjugated entity, part of a fusion protein, nanoparticles, or cells that are pulsed with the antigen. Ideally, a tolerance-inducing drug is based on the drug it tries to tolerize against, and can easily be modified.

Disclosed herein are compositions and methods for inducing tolerance towards therapeutic proteins, e.g., protein-based drugs. In some aspects, the present disclosure provides therapeutic, polymer-tethered antigens that include mannose monomers or derivatives thereof. In some embodiments, derivatives of mannose include optionally substituted mannose substituents. In several embodiments, the mannose derivative comprises a mannose with a phosphate at the C1, C2, C3, C4, C5, and/or C6 position. In several embodiments, the targeting moiety comprises mannose-6-phosphate. In some embodiments, compositions including a mannose or mannose-derived moiety induce tolerance to immunogenic protein-based therapeutics, delete antigen-specific CD4 and CD8 T cells, elevate levels of regulatory T cell responses and IL-10 producing Breg cells, and/or reduce antigen-specific plasma cells and memory B cells. The compositions disclosed herein may be targeted for delivery to antigen-presenting cells including, but not limited to, hepatocytes, LSECs, Kupffer cells, and stellate cells. In some aspects, the compositions disclosed herein exhibit affinity for and specifically bind to mannose-binding receptors.

In several embodiments, there are provided compounds, as well as compositions comprising such compounds, of Formula 1:

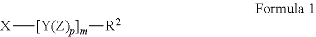

where X comprises an antigen, a tolerogenic portion thereof, or a mimetic thereof, Y comprises a linker moiety, Z comprises a moiety that specifically targets a mannose receptor, p is an integer from about 2 to about 250, m is an integer from about 1 to about 100, and R² is any of functional groups I-III:

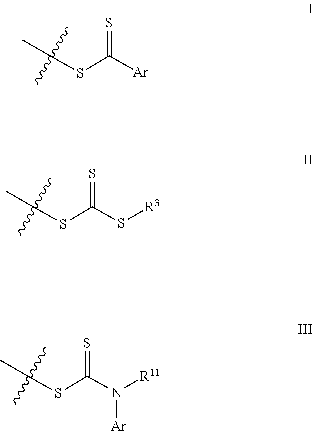

where Ar is a substituted or unsubstituted aromatic group, where R³ is any carbon-containing linear or heterocyclic moiety, and R¹¹ is hydrogen or an alkyl group.

In several embodiments, the moiety that specifically targets a mannose receptor is selected from the group consisting of α-linked mannose, β-linked mannose, substituted mannose, mannose-6-phosphate, N-acetyl mannosamine, and mannan having β(1-4), α(1-6), α(1-2), and/or α(1-3) linkages. In several embodiments, a plurality of these targeting moieties are used in combination in order to enhance targeting of and/or binding to the mannose receptor.

In several embodiments, Y is a linker resulting from reaction of at least one of a N-hydroxysuccinamidyl linker, maleimide linker, PEG linker, vinylsulfone linker, pyridyl di-thiol-poly(ethylene glycol) linker, pyridyl di-thiol linker, n-nitrophenyl carbonate linker, NHS-ester linker, and nitrophenoxy poly(ethylene glycol)ester linker. In some embodiments, Y is covalently bound to X.

Depending on the embodiment, the following portion of Formula 1 (—[Y(Z)p]-) is represented by one of Formula Ya to Yr:

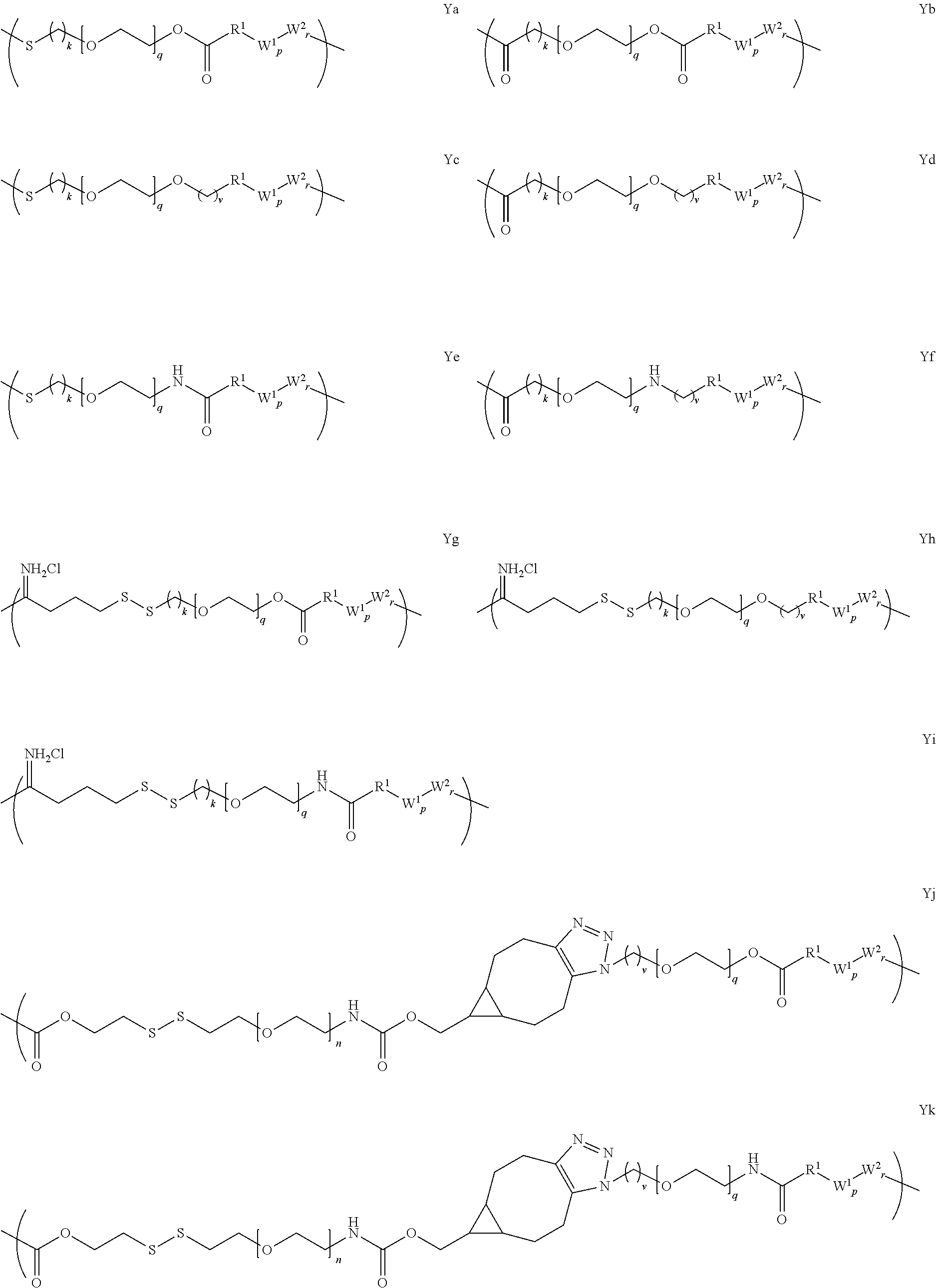

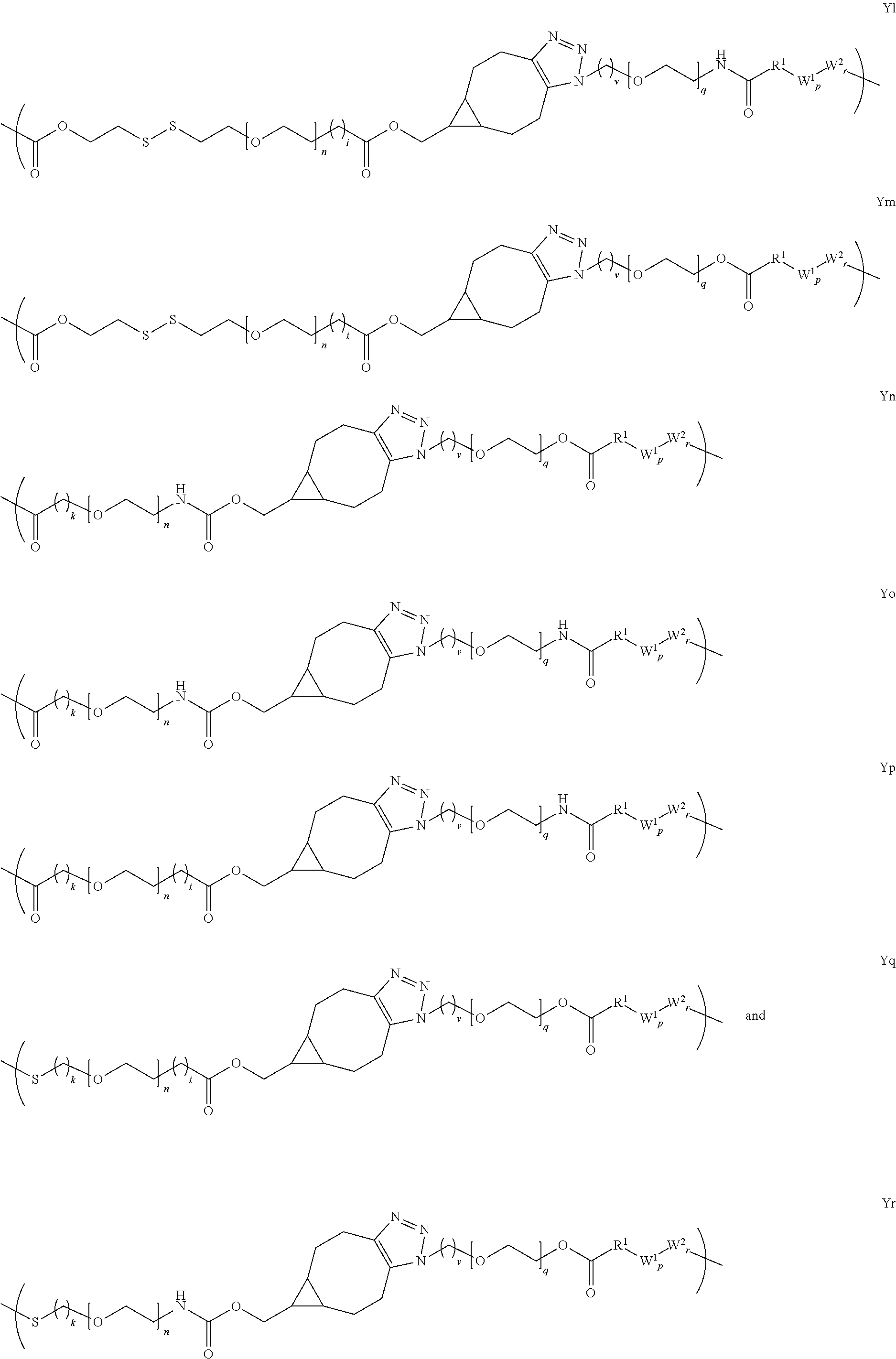

where
   n is an integer from about 1 to about 100;
   q is an integer from about 1 to about 44;
   k is an integer from about 1 to about 12;
   i is an integer from 0 to about 20;
   v is an integer from about 1 to about 4;
   p is an integer from about 2 to about 250;
   r is an integer from 0 to about 250;
   R₁ is —CH₂—, —(CH₂)₂—C(CH₃(CN)—, —(CH₂)₂—C(CH₃)(CH₃)—, —(CH₂)₂—CH(CH₃)— or —CH(CH₃)—;
   W¹ and W² are as defined below:

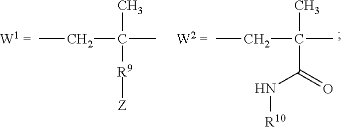

R⁹ is a direct bond, —(CH₂)₂—NH—C(O)— (an ethylacetamido group or “EtAcN”) or —(CH₂)₂—(O—CH₂—CH)—NH—C(O)— (a pegylated ethylacetamido group or “Et-PEGt-AcN”)
t is an integer from 1 to 5, Z is mannose or a mannose receptor-targeting moiety; and R¹⁰ is an aliphatic group, an alcohol, an aliphatic amine-containing group, or an aliphatic alcohol.

In several embodiments, Y is an antibody, antibody fragment, peptide or other ligand that binds to X.

In several embodiments, X is an antigen against which a patient may develop or has developed an unwanted immune response. For example, depending on the embodiment, the antigen may be a foreign transplant antigen, an alloantigen, an autoimmune antigen, a food antigen, an animal antigen, a plant antigen, an environmental antigen, a therapeutic antigen, a synthetic self-antigen, or a tolerogenic (e.g., immunogenic, or capable of inducing an immune response) portion thereof. In several embodiments, X is an asparaginase antigen or an ovalbumin antigen. In several embodiments, the antigen is comprised in a vesicle, cell fragment, or cell.

In several embodiments, the antigen comprises at least one autoimmune antigen or tolerogenic portion thereof. In several embodiments, the at least one autoimmune antigen comprises at least one of an immunogenic fragment or fragments of myelin basic protein (MPB), an immunogenic fragment or fragments of myelin oligodendrocyte glycoprotein (MOG), an immunogenic fragment or fragments of myelin proteolipid protein (PLP), MBP, MOG, or PLP. In several embodiments, the at least one autoimmune antigen comprises at least one of SEQ ID Nos. 23-47. In several embodiments, the at least one autoimmune antigen comprises at least one of SEQ ID Nos. 24, 25, 27, 28, 31, 32, 33, 34, 35, 36, 43, 44, 45, 46, and 47. In some embodiments, the compound optionally further comprises at least one of SEQ ID NOs: 29, 38, 39, 40, 41, and 42. In several embodiments, such compounds are for use in treatment of or prevention of multiple sclerosis. In some embodiments, such compounds are administered for the use of preventing multiple sclerosis in a subject predicted to have multiple sclerosis. In some aspects, such compounds are administered to a subject presenting one or more symptoms of multiple sclerosis.

In several embodiments, the at least one autoimmune antigen comprises at least one of insulin, proinsulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5, or an immunogenic fragment of any of these antigens. In several embodiments, the autoimmune antigen comprises an immunogenic fragment of proinsulin. Optionally an immunogenic fragment of IA-2, GAD-65, GAD-67, insulin, and/or IGRP is included. In several embodiments, the at least one autoimmune antigen comprises at least one of SEQ ID NOs: 1-19, or an immunogenic fragment of any of SEQ ID NOs: 1-19. In several embodiments, the at least one autoimmune antigen comprises at least one of SEQ ID NOs: 4-19. In several embodiments, such compounds are for use in the treatment or prevention of Type 1 Diabetes. In some embodiments, such compounds are administered for the use of preventing Type 1 Diabetes in a subject predicted to have Type 1 Diabetes. In some aspects, such compounds are administered to a subject presenting one or more symptoms of Type I Diabetes.

In several embodiments, the antigen comprises a food antigen, or a tolerogenic portion thereof. In several embodiments, the antigen comprises at least one of tissue transglutaminase, high molecular weight glutenin, low molecular weight glutenin, gluten, alpha-gliadin, gamma-gliadin, omega-gliadin, hordein, secalin, avenin, and deamidated forms thereof. In several embodiments, the antigen comprises a tolerogenic portion of at least one of tissue transglutaminase, high molecular weight glutenin, low molecular weight glutenin, gluten, alpha-gliadin, gamma-gliadin, omega-gliadin, hordein, secalin, avenin, and deamidated forms thereof. In several embodiments, the antigen comprises at least one of SEQ ID NOs. 54-61. In several embodiments, such compounds are for use in the treatment or prevention of Celiac Disease. In some embodiments, such compounds are administered for the use of preventing Celiac Disease in a subject predicted to have Celiac Disease. In some aspects, such compounds are administered to a subject presenting one or more symptoms of Celiac Disease.

In several embodiments, the mannose receptor is mannose-6-phosphate receptor. In several embodiments, Y and X are connected through a bond configured to cleave when the compound reaches a target area. Advantageously, this cleavage, in several embodiments, is triggered when the compound is at a target site (e.g., site where the mannose receptor is bound). This allows, in several embodiments, delivery of the free antigen to the target site.

In several embodiments, Ar is selected from:

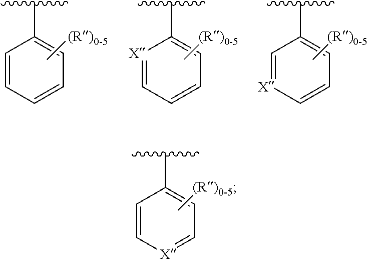

where each instance of R″, when present, is independently selected from an optionally substituted C1-6-alkyl, optionally substituted C1-6 alkoxy, optionally substituted amino, OH, or halogen and wherein, X″ is a heteroatom. In several embodiments, X″ is N. In several embodiments, R11 is C1-6-alkyl. In several embodiments, R11 is —CH3. In several embodiments, R3 is C1-6-alkyl.

There are also provided herein, compositions comprising the compounds as described above, or elsewhere herein. Likewise, there are provided for herein uses of these compounds for inducing tolerance (or treating an unwanted immune response) to an antigen, a tolerogenic portion (or portions) of one or more antigens, and/or to mimetics of the antigens or portions of antigens. Also provided are uses of such compounds in the preparation of a medicament for inducing tolerance (or treating an unwanted immune response) to an antigen, a tolerogenic portion (or portions) of one or more antigens, and/or to mimetics of the antigens or portions of antigens.

Certain aspects of the disclosure are directed towards compositions comprising a compound of Formula 1:

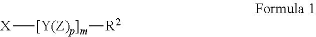

where X comprises an antigen or a tolerogenic portion thereof, Y comprises a linker moiety, Z comprises a moiety that specifically targets a mannose receptor, p is an integer from about 2 to about 250, m is an integer from about 1 to about 100, R² is any of functional groups I-III:

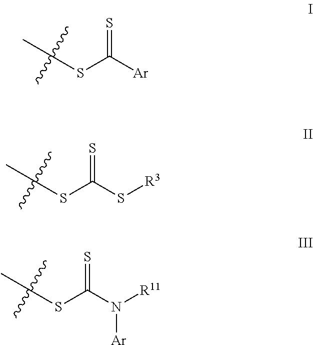

where Ar is a substituted or unsubstituted aromatic group, R³ is any carbon-containing linear or heterocyclic moiety, and R¹¹ is hydrogen or an alkyl group. In some embodiments, R² comprises an end-capping group. In some embodiments, R² when disconnected from the construct, forms a stable or substantially stable free radical. In some embodiments, R² is a reversible addition-fragmentation chain transfer (RAFT) agent for a living polymerization. In some embodiments, R² can be reversibly added and removed to the construct to lengthen the linker region. In some embodiments, R² is a RAFT agent. In some embodiments, R² is not a RAFT agent. In some embodiments, R² is H or is absent. In some embodiments, R² is an optionally substituted dithiobenzoate, a trithiocarbonate, or a xanthate. In some embodiments, R³ or R¹¹ may be hydrogen, optionally substituted alkyl, optionally substituted cycloalkyl, optionally substituted aryl, or optionally substituted heteroaryl. In some embodiments, In some embodiments, R³ is hydrogen, optionally substituted C₆-aryl, or C₁₋₆-alkyl (optionally substituted with halogen, or hydroxyl). In some embodiments, Ar as provided above is phenyl (optionally substituted with one or more OH groups, NH₂ groups, and/or halogens). In some embodiments, R² is one of the functional groups:

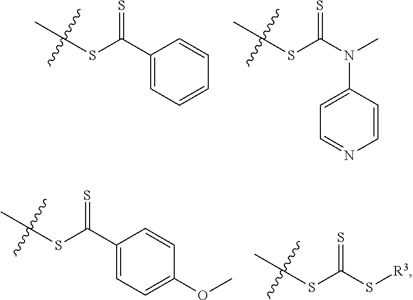

where R³ is as defined above. In several embodiments, Ar in any of functional groups I or II is an optionally substituted C₆-C₁₄ aryl or an optionally substituted heteroaryl having 6 to 14 ring members of which 1-4 are heteroatoms. In several embodiments, the optionally substituted C₆-C₁₄ aryl is optionally substituted with one or more functional groups selected from C₁-C₆alkyl, amino, halogen, —OH, or combinations thereof. In several embodiments, Ar is selected from the group consisting of:

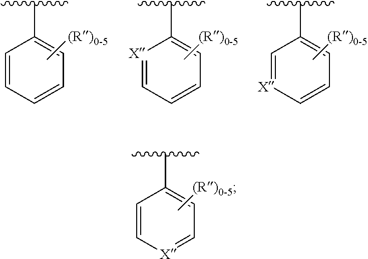

where each instance of R″, when present, is independently selected from an optionally substituted C₁₋₆-alkyl, optionally substituted C₁₋₆ alkoxy, optionally substituted amino, OH, or halogen. In several embodiments, X″ is a heteroatom. In several embodiments, X″ is N. In several embodiments, R¹¹ is C₁₋₆-alkyl. In several embodiments, R¹¹ is —CH₃. In several embodiments, R³ is C₁₋₆-alkyl.

In some embodiments, Y and X are connected via a bond that cleaves or is configured to cleave at a target site for the compound. In several embodiments, Y and X are bonded through a disulfanyl ethyl ester or a disulfide bond. In several embodiments, the bond between Y and X is configured to cleave when the compound of Formula 1 reaches its biological target (e.g., the liver, liver cells, and/or the cytosol of cells in the liver) in a patient. In several embodiments, the Y—X bond cleaves in the presence of a cellular reducing agent (e.g., glutathione). In some embodiments, advantageously, once the bond between X and Y is cleaved, X is left in its native form and/or an active form. In some embodiments, once X is cleaved from Y, it is in a form that is more active than when bound to Y. In some embodiments, di-thiol-containing compounds, particularly disulfanylethyl carbamate-containing links between X and Y (named including a free amine of X, otherwise named a “disulfanyl ethyl ester” without including the free amine of X) are advantageous as having the ability to cleave and release an antigen in its original form once inside a cell, for example as illustrated below (where Y′ indicates the remaining portion of the linker and X and Z are as defined)

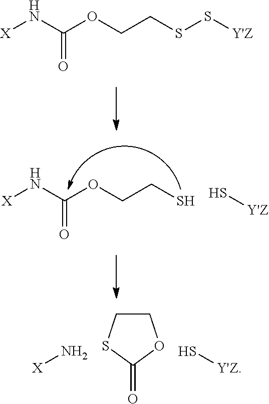

In several embodiments, the liver targeting moiety of the compound of Formula 1 is not galactose, galactosamine, N-acetylgalactosamine, glucose, glucosamine and/or N-acetylglucosamine.

In some embodiments, Y is a linker. In several embodiments, Y is a reaction product resulting from one or more reactions involving at least one of the following: N-hydroxysuccinamidyl (NHS) linker, NHS ester linker, PEG linker, maleimide linker, vinylsulfone linker, pyridyl di-thiol-poly(ethylene glycol) linker, pyridyl di-thiol linker, n-nitrophenyl carbonate linker, or a nitrophenoxy poly(ethylene glycol)ester linker. The linker may have one or more mannose moieties or mannose receptor-targeting moieties bound to it. In several embodiments, Y comprises an antibody, an antibody fragment, a peptide, or a disulfanyl ethyl ester to which one or more mannose moieties or mannose receptor-targeting moieties are bound. In some aspects, —[Y(Z)ₚ]— is a group represented by one of Formula Ya to Yr:

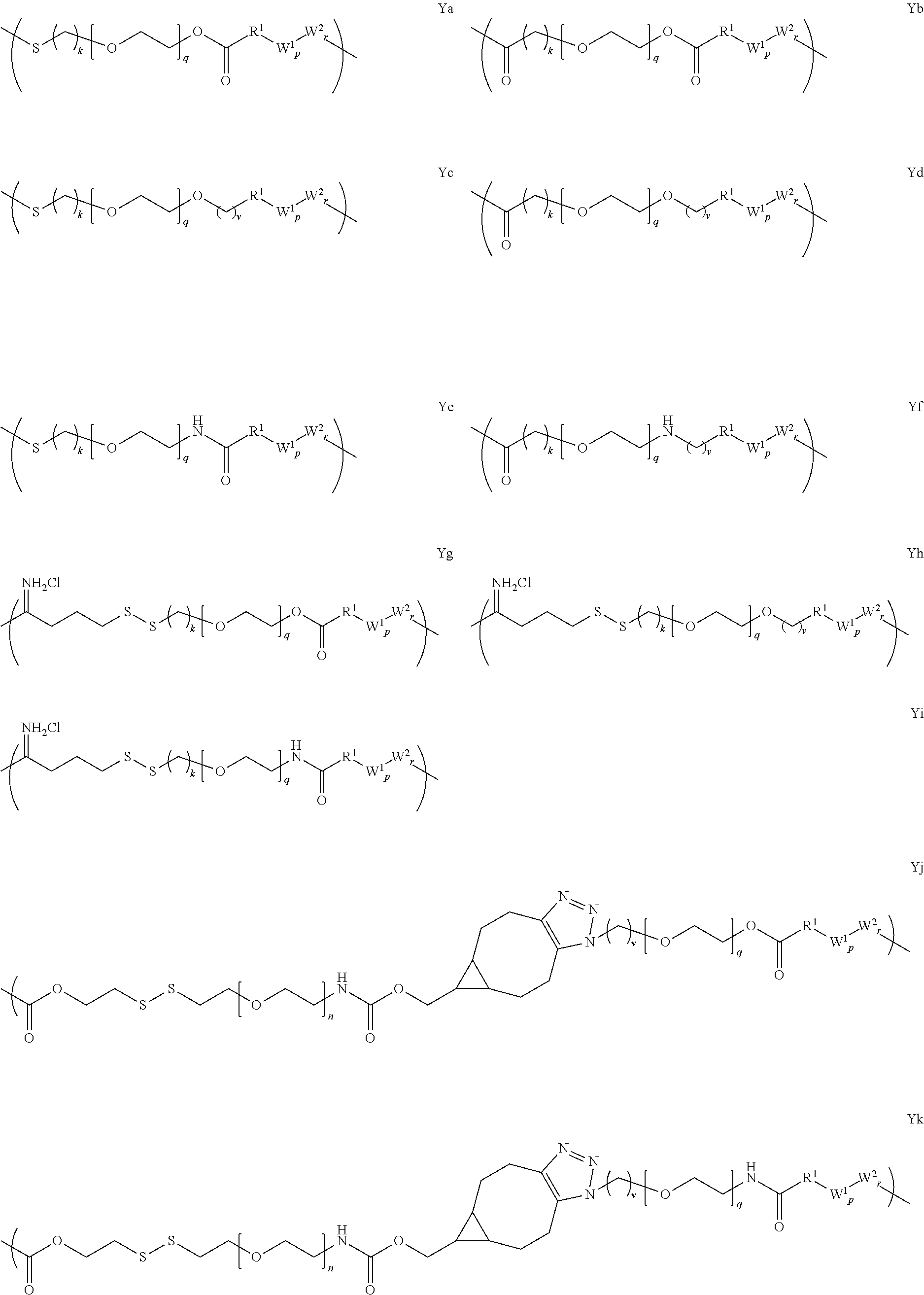

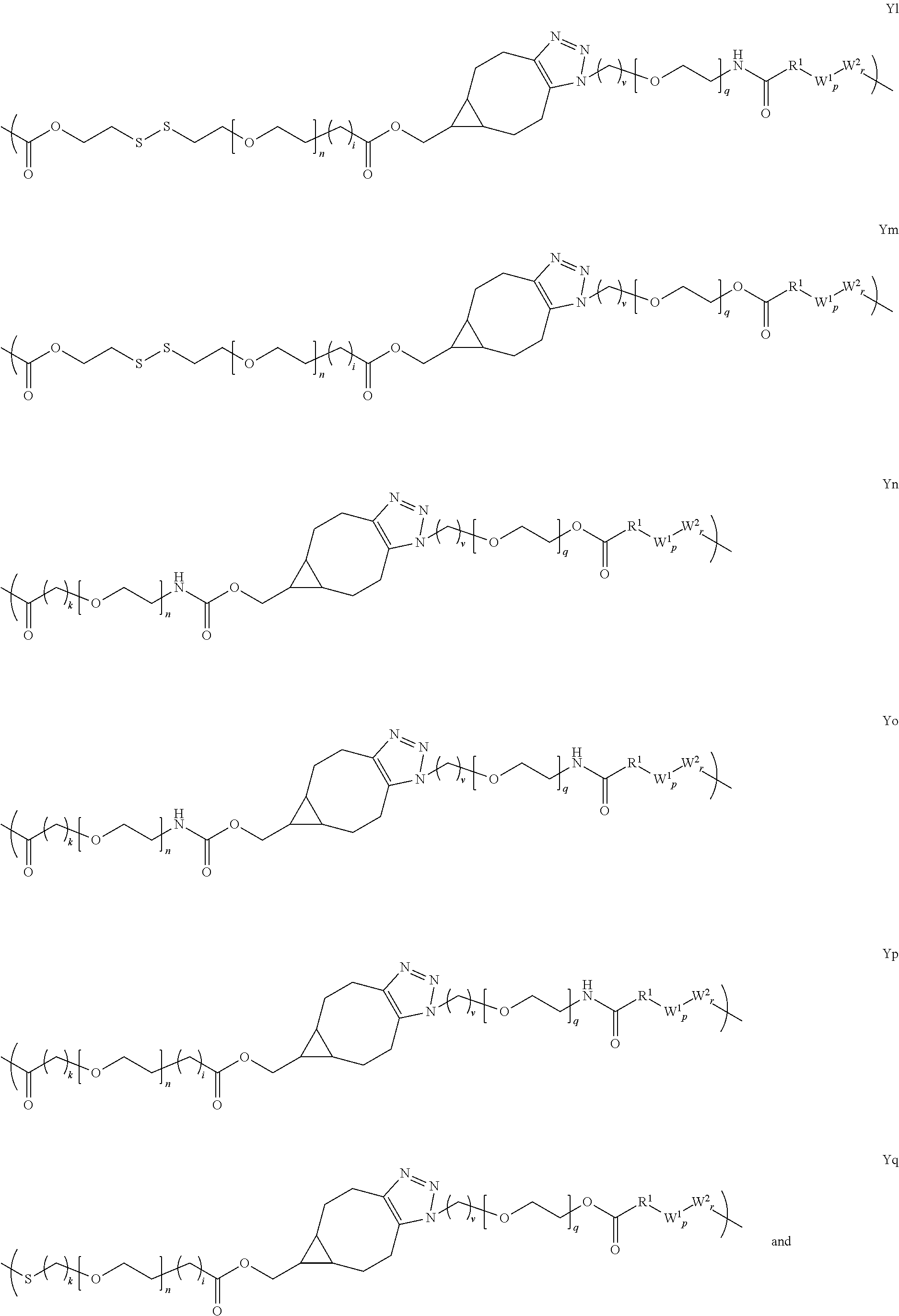

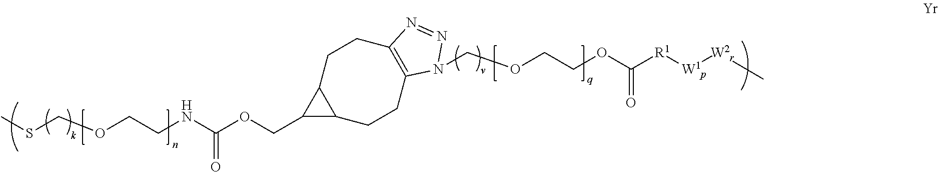

where the left, opening parentheses “(” signifies the location of the bond between X and Y, the right, closing parentheses “)” signifies the location of the bond between Y and R². In several embodiments, n is an integer greater than or equal to about: 1, 10, 20, 40, 50, 75, 100, 150 or ranges including and/or spanning the aforementioned values. In several embodiments, n is an integer greater than or equal to about: 1, 2, 3, 4, 5, 10, 15, 20, 30, 40, 50, or ranges including and/or spanning the aforementioned values. In several embodiments, q is an integer greater than or equal to about: 1, 10, 20, 40, 50, 75, 100, 150 or ranges including and/or spanning the aforementioned values. In several embodiments, q is an integer greater than or equal to about: 1, 2, 3, 4, 5, 10, 15, 20, 30, 40, 50, 75, 100, 150, or ranges including and/or spanning the aforementioned values. In several embodiments, k is an integer greater than or equal to about: 1, 2, 3, 4, 5, 10, 15, 20, or ranges including and/or spanning the aforementioned values. In several embodiments, v is an integer greater than or equal to about: 1, 2, 3, 4, 5, 10, 15, 20, or ranges including and/or spanning the aforementioned values. In several embodiments, k is 2. In several embodiments, v is 2. In several embodiments, n is 4. In several embodiments, n is 44. In several embodiments, q is 3. As used herein, variables disclosed as having structure, a value, or a range of values for one embodiment, may also have those values when the variable is used in another embodiment (even where the variable is not defined with respect to that other embodiment). In several embodiments, n is an integer from 1 to 100. In several embodiments, q is an integer from 1 to 44. In several embodiments, k is an integer from 1 to 12. In several embodiments, i is an integer from 0 to 20. In several embodiments, v is an integer from 1 to 4. In several embodiments, R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—. In several embodiments, W¹ and W² are as depicted below:

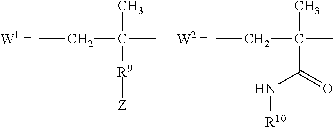

where Z is mannose or a mannose receptor-targeting moiety, R⁹ is a direct bond, —(CH₂)₂—NH—C(O)— (an ethylacetamido group or “EtAcN”) or —(CH₂)₂—(O—CH₂—CH)ₜ—NH—C(O)— (a pegylated ethylacetamido group or “Et-PEGₜ-AcN”), t is an integer from 1 to 5, p is an integer from 2 to 250, R¹⁰ is an aliphatic group, an alcohol, an aliphatic amine-containing group, or an aliphatic alcohol, and r is an integer from 0 to 250. In several embodiments, —W¹ₚ—W²ᵣ— (e.g., as provided in —[Y(Z)ₚ]— or in linker structures) is a random copolymer or block copolymer of W₁ and W₂. In several embodiments, the number of repeat units of W¹ is denoted as p and wherein p is an integer of at least about 1. In several embodiments, the number of repeat units of W² is denoted as r and wherein r is an integer of at least about 1. In some embodiments, R¹⁰ is a C_{f}alkyl or C_{f}alkylOH_{B}, where f represents the number of carbons in the alkyl group and is an integer between 0 and 10, and g represents the number of hydroxyl groups present on the alkyl group and is an integer between 0 and 10. In some embodiments, R¹⁰ is 2-hydroxyethyl. In some aspects —W¹ₚ—W²_{q}— represents a block copolymer or a random copolymer of W¹ and W² monomers.

In several embodiments, the linker comprises a polymeric chain with pendant liver targeting moieties decorating the polymeric chain. In some embodiments, the polymeric chain (or Y) comprises Y′ as disclosed elsewhere herein. In several embodiments, the polymeric chain comprises an acrylate portion (e.g., acrylate-based polymers and/or acrylate-based copolymers). In several embodiments, the acrylate portion comprises one or more acrylate units (e.g., acrylate derivatives, including methacrylates and derivatives thereof) comprising a pendant liver targeting agent. In several embodiments, the polymeric chain comprises a hydrophilic portion and/or region. In several embodiments, the hydrophilic portion comprises a length of one or more regions having —(CH₂CH₂O)ₛ— where s is an integer from about 1 to about 44. In several embodiments, s is an integer greater than or equal to about: 1, 2, 3, 4, 5, 10, 15, 20, 30, 40, 50, 75, 100, 150, or ranges including and/or spanning the aforementioned values. In some embodiments, the hydrophilic portion comprises one or more polyethylene glycol (PEG) regions. In some embodiments, the PEG may have polydispersity as measured by the weight average molecular weight in g/mol (Mw) of the PEG divided by the number average molecular weight in g/mol (Mw) of the PEG (e.g., Mw/Mn). In some embodiments, the PEG chains have a number average or weight average molecular weight (g/mol) of equal to or at least about: 500, 1000, 2000, 5000, 10000, or ranges including and/or spanning the aforementioned values. In several embodiments, the polymeric chain is optionally substituted. In some embodiments, the polymeric chain comprises pendant hydrophilic groups such as a —OH, —SO(OH)₂, optionally substituted polyether, optionally substituted polyamino, and the like.

In several embodiments, the antigen and liver targeting portion of the compound are joined using click chemistry, for example, by functionalizing the antigen with a first linker arm comprising an alkynyl group (or an azide), functionalizing the liver targeting moiety with a second linker arm comprising an azide (or an alkynyl group), and clicking them together via “click” chemistry. In some embodiments, an alkynyl group that can be clicked in copper-free conditions is used. In some embodiments, —[Y(—Z)ₚ]— is a group represented by one or more of Formulae AI-AIV:

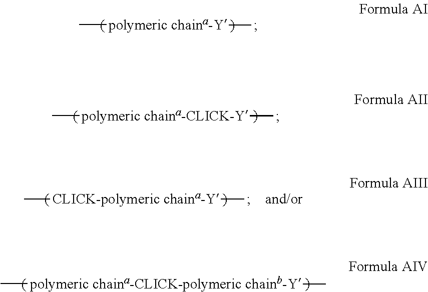

where the left, opening parentheses “(” signifies the location of the bond between X and Y, the right, closing parentheses “)” signifies the location of the bond between Y and R², Y′ is a random copolymer or block copolymer of two or more different types of repeat units, wherein at least one type of repeat unit comprises a pendant Z group, (or plurality of pendant Z groups) where Z is mannose and/or a mannose receptor-targeting moiety. In some embodiments, Y′ is a random copolymer or block copolymer of W¹ and W², where W¹ and W² are as depicted below:

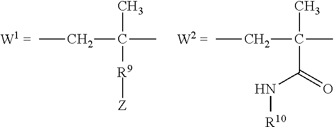

where Z is mannose and/or a mannose receptor-targeting moiety (including, but not limited to, one or more of mannosamine, N-acetylmannosamine, or N-acetylglucosamine), R⁹ is a direct bond, optionally substituted —C(O)—NH—(CH₂)₂— (an ethylacetamido group or “EtAcN”) or optionally substituted —C(O)—NH—(CH₂)₂—(O—CH₂—CH)ₜ— (a pegylated ethylacetamido group or “Et-PEGₜ-AcN”), t is an integer from 1 to 5. In some embodiments, t is an integer of equal to or at least about: 1, 2, 3, 4, 5, 10, 20, or ranges including and/or spanning the aforementioned values. In several embodiments, R¹⁰ is an aliphatic group, an alcohol, an aliphatic amine-containing group, or an aliphatic alcohol. In some embodiments, R⁹ or R¹⁰ are independently optionally substituted alkyl, an optionally substituted polyether, or optionally substituted polyamino. In some embodiments, R¹⁰ is an optionally substituted C_{f}alkyl, optionally substituted C_{f}alkylOH_{g}, or an optionally substituted —(C_{f}alkylOH_{g})—O)ₑ—H where f represents the number of carbons in the alkyl group and is an integer between 0 and 10, g represents the number of hydroxyl groups present on the alkyl group and is an integer between 0 and 10, and e represents the number of alkyl/ether repeat units and is an integer between 0 and 10. In some embodiments, e, f, and g are independently selected integers of equal to or at least about: 0, 1, 2, 3, 4, 5, 10, or ranges including and/or spanning the aforementioned values. In some embodiments, R¹⁰ is a 2-hydroxyethyl (e.g., —CH₂CH₂OH). In some embodiments, R¹⁰ is an optionally substituted 2-hydroxyethyl. In some embodiments, R¹⁰ is an optionally substituted polyether.

In some embodiments, Y′ is represented as —W¹ₚ—W²ᵣ—. As noted elsewhere herein, —W¹ₚ—W²ᵣ— may represent a block copolymer or a random copolymer of W¹ and W² monomers having p repeat units of W¹ and r repeat units of W². In some embodiments, p is an integer equal to or greater than about: 1, 50, 85, 100, 150, 165, 200, 225, 250, 300, 400, or ranges including and/or spanning the aforementioned values. In some embodiments, r is an integer equal to or greater than about: 1, 50, 85, 100, 150, 165, 200, 225, 250, 300, 400, or ranges including and/or spanning the aforementioned values. In some embodiments, Y′ is a homopolymer of W¹ or W². In some embodiments, r is 0. In some embodiments, the sum of p and r is an integer equal to or greater than about: 1, 50, 85, 100, 150, 165, 170, 200, 225, 250, 300, 400, 600, 800, or ranges including and/or spanning the aforementioned values.

In some embodiments, polymeric chain^{a} and polymeric chain^{b} are present or optionally not present. In some embodiments, where present, polymeric chain^{a} and polymeric chain^{b} can independently comprise hydrophilic polymers. In some embodiments, where present, polymeric chain^{a} and polymeric chain^{b} can independently comprise one or more optionally substituted —(CH₂CH₂O)ₛ—, optionally substituted —(CH₂)ᵤ—, or optionally substituted alkylene. In several embodiments, u is an integer less than or equal to about: 1, 5, 10, 20, or ranges including and/or spanning the aforementioned values. In some embodiments, polymeric chain^{a} and polymeric chain^{b} comprise or consist of one or more of the following structures, or a portion thereof:

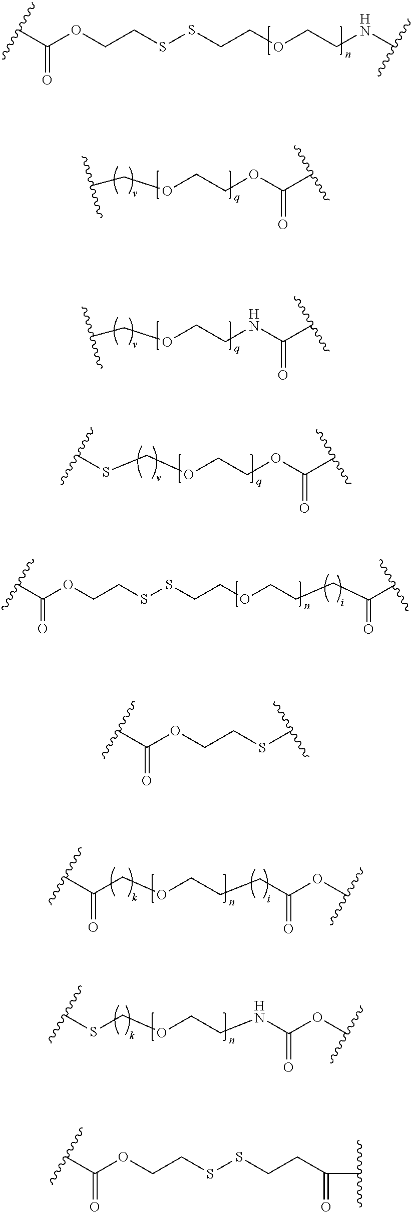

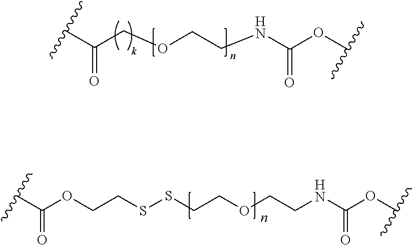

wherein the variables (e.g., i, k, n, q, v, etc.) are as disclosed elsewhere herein. In several embodiments, for example, n is an integer from about 1 to about 100, q is an integer from about 1 to about 100, k is an integer from about 1 to about 20, i is an integer from about 0 to about 20, and v is an integer from about 1 to about 20. In several embodiments, n or q represents the number of repeat units in a PEG chain. In some embodiments, the PEG chain may have some polydispersity. In some embodiments, n and q do not indicate a number of repeat units but instead independently indicate the presence of a PEG polymer chain having a Mn (in g/mol) or Mw (in g/mol) of equal to or at least about 500, 1000, 2000, 5000, 10000, or ranges including and/or spanning the aforementioned values. In some embodiments, k, i, and v can each independently comprise an optionally substituted alkylene.

In several embodiments, n is an integer greater than or equal to about: 1, 10, 20, 40, 50, 75, 100, 150 or ranges including and/or spanning the aforementioned values. In several embodiments, n is an integer greater than or equal to about: 1, 2, 3, 4, 5, 10, 15, 20, 30, 40, 50, or ranges including and/or spanning the aforementioned values. In several embodiments, q is an integer greater than or equal to about: 1, 10, 20, 40, 50, 75, 100, 150 or ranges including and/or spanning the aforementioned values. In several embodiments, q is an integer greater than or equal to about: 1, 2, 3, 4, 5, 10, 15, 20, 30, 40, 50, 75, 100, 150, or ranges including and/or spanning the aforementioned values. In several embodiments, k is an integer greater than or equal to about: 1, 2, 3, 4, 5, 10, 15, 20, or ranges including and/or spanning the aforementioned values. In several embodiments, v is an integer greater than or equal to about: 1, 2, 3, 4, 5, 10, 15, 20, or ranges including and/or spanning the aforementioned values. In several embodiments, k is 2. In several embodiments, v is 2. In several embodiments, n is 4. In several embodiments, n is 44. In several embodiments, q is 3. As used herein, variables disclosed as having structure, a value, or a range of values for one embodiment, may also have those values when the variable is used in another embodiment (even where the variable is not defined with respect to that other embodiment).

In several embodiments, the “CLICK” group and/or —[Y(—Z)ₚ]—, more generally, comprises the following functional unit:

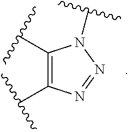 [SMILES: *c1nnn(*)c1*]

In several embodiments, the “CLICK” group and/or —[Y(—Z)ₚ]—, more generally, comprises one or more of the following units (each of which may be optionally substituted):

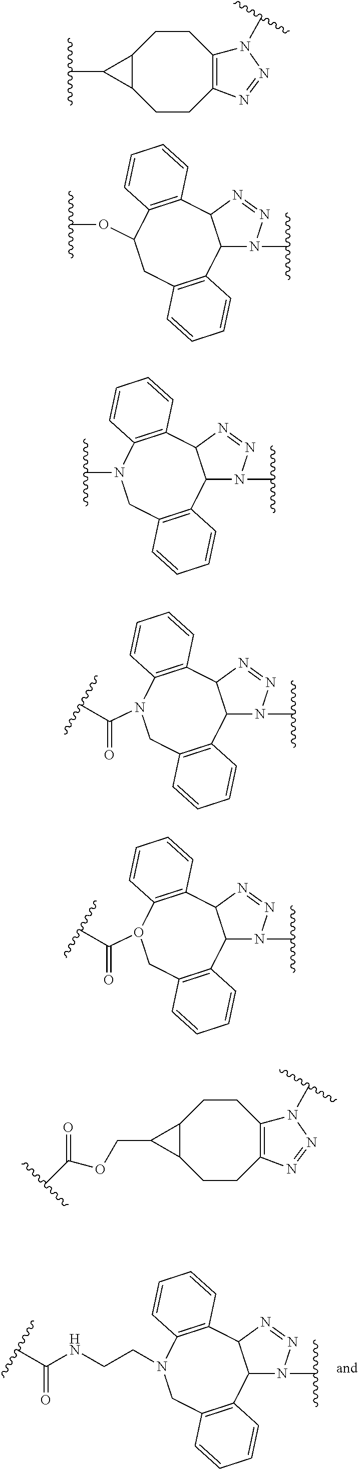

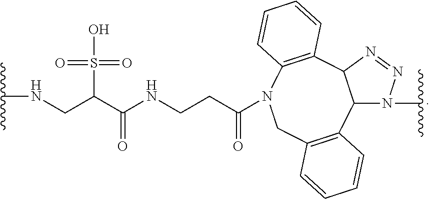 [SMILES: *NCC(C(=O)NCCC(=O)N1Cc2ccccc2C2C(N=NN2*)c2ccccc21)S(=O)(=O)O]

In several embodiments, —[Y(—Z)p]- comprises the one or more of the following functional units:

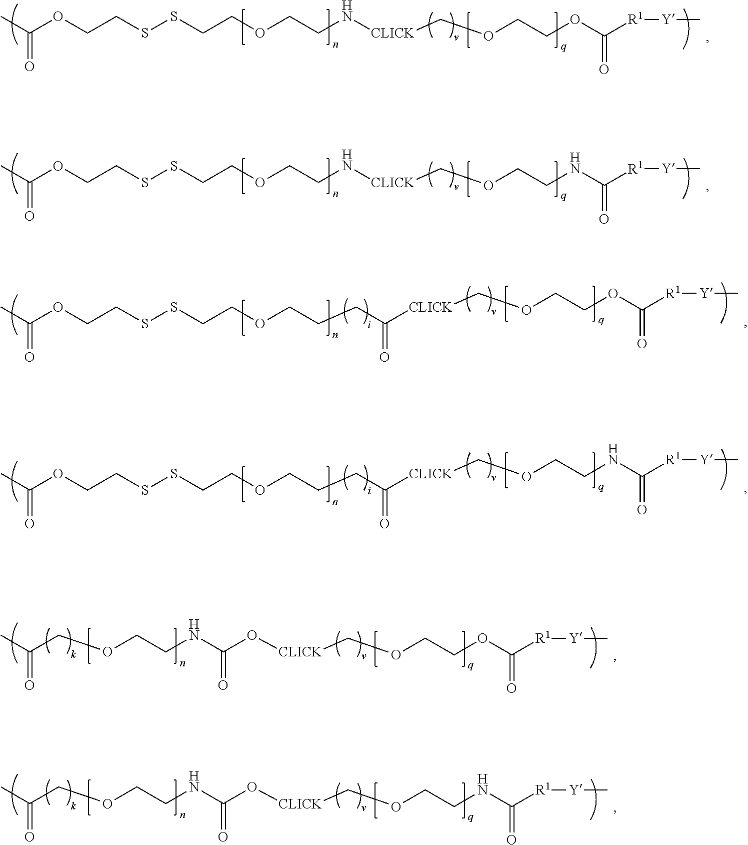

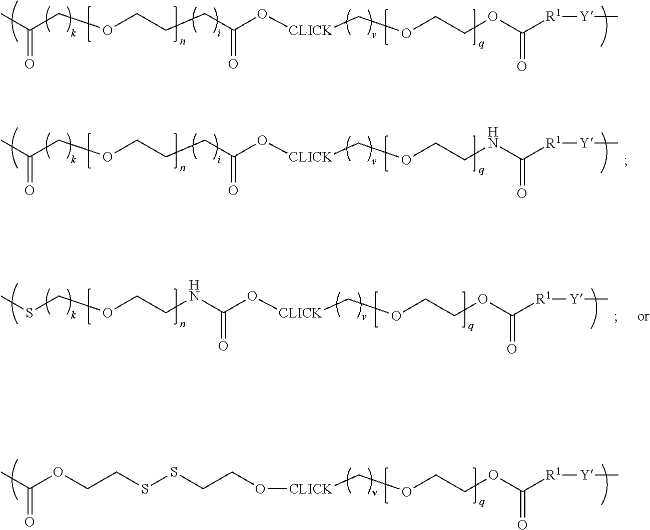

wherein each variable (e.g., i, k, n, q, v, CLICK, R¹, Y′, etc.) is as disclosed elsewhere herein. In some embodiments, for example, n is an integer from about 1 to about 44, q is an integer from about 1 to about 44, k is an integer from about 1 to about 12, i is an integer from about 0 to about 20, v is an integer from about 1 to about 4, and R₁ is —CH₂—, —(CH₂)₂—C(CH₃)(CN)—, —(CH₂)₂—C(CH₃)(CH₃)—, —(CH₂)₂—CH(CH₃)—, —CH(CH₃)—, or is absent.

In several embodiments, —[Y(Z)ₚ]— is a group represented by any one or more of Formula Ya′ to Yr′:

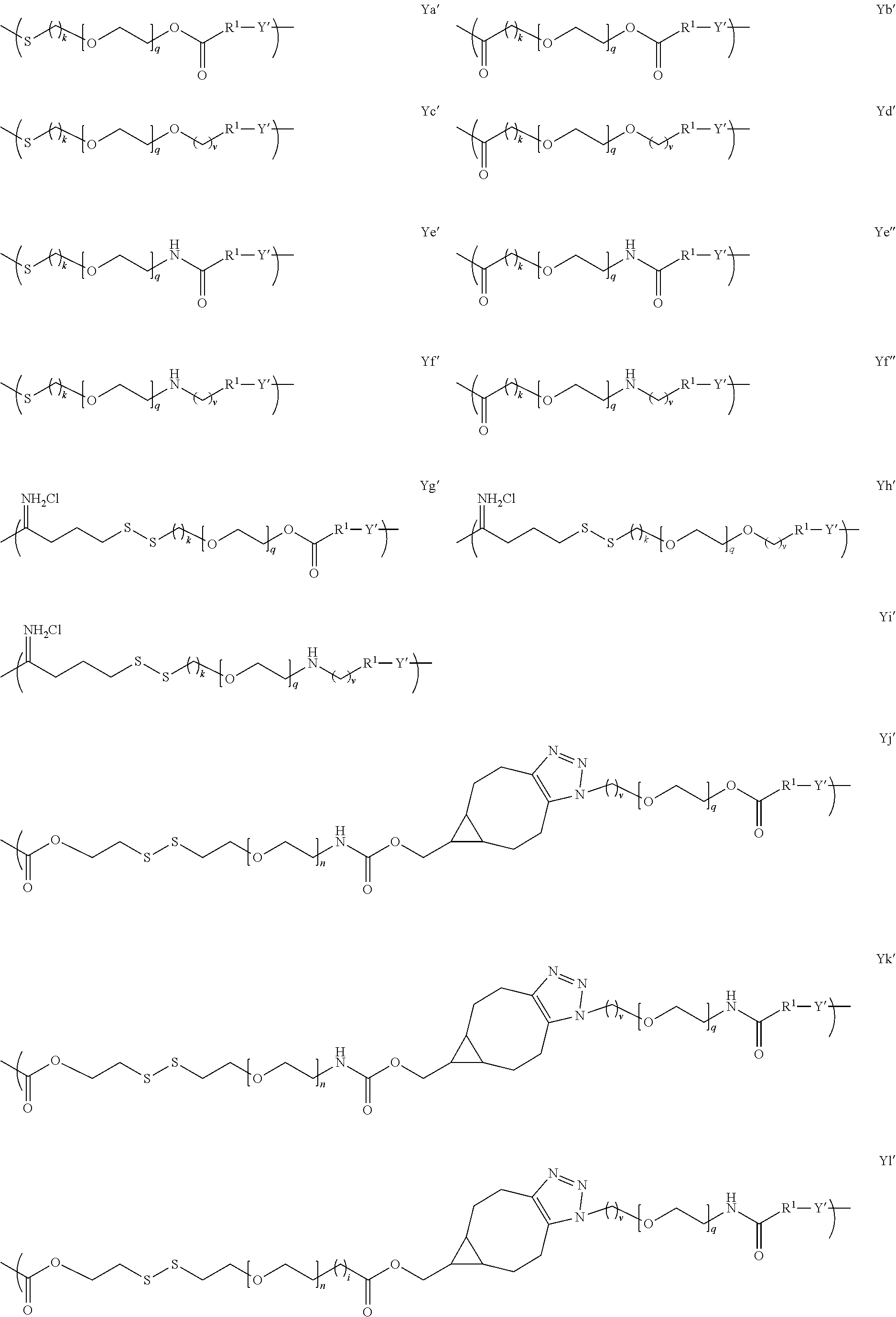

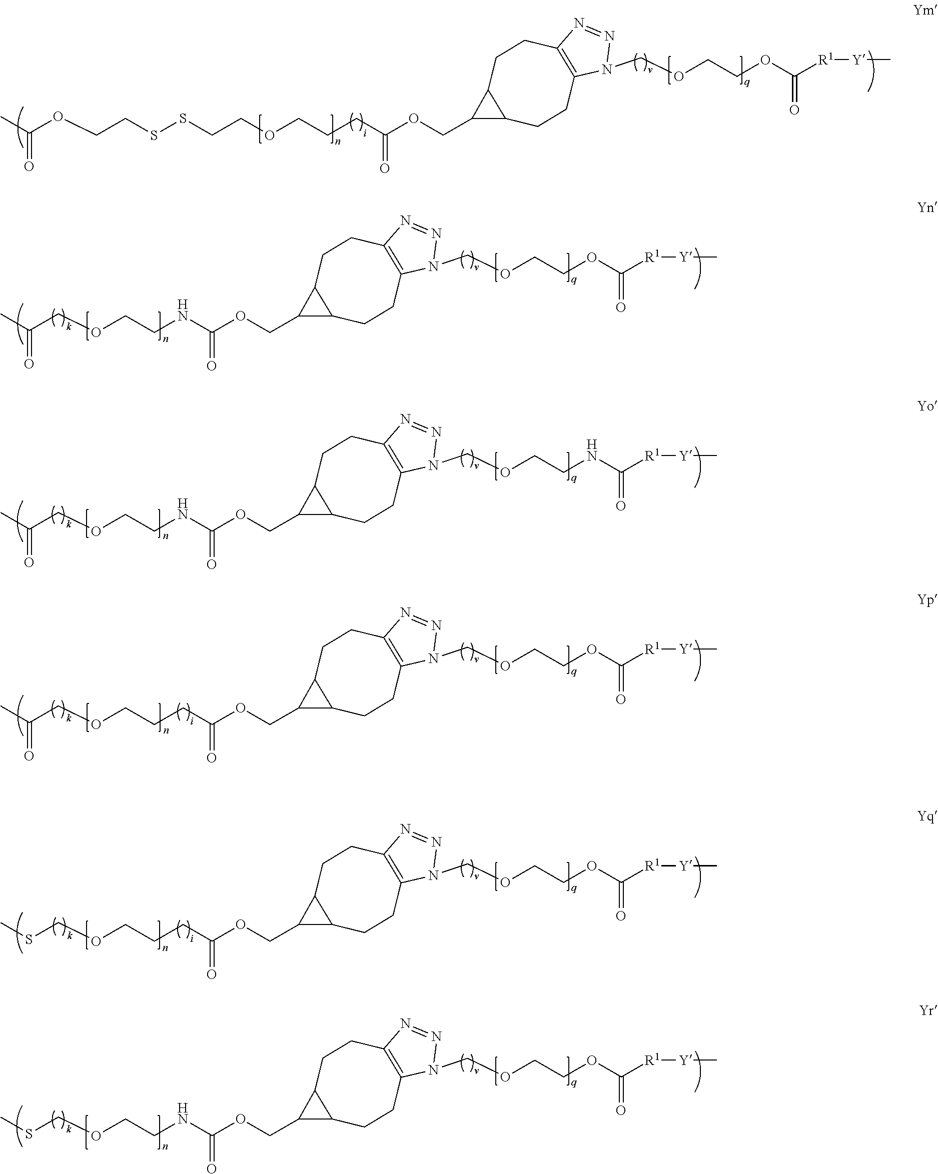

wherein the variables (e.g., i, k, n, q, v, R¹, Y′, etc.) are as disclosed elsewhere herein. For example, in several embodiments, n is an integer greater than or equal to about: 1, 2, 3, 4, 5, 10, 15, 20, 30, 40, 50, or ranges including and/or spanning the aforementioned values. In several embodiments, q is an integer greater than or equal to about: 1, 2, 3, 4, 5, 10, 15, 20, 30, 40, 50, 75, 100, 150, or ranges including and/or spanning the aforementioned values. In several embodiments, k is an integer greater than or equal to about: 1, 2, 3, 4, 5, 10, 15, 20, or ranges including and/or spanning the aforementioned values. In several embodiments, v is an integer greater than or equal to about: 1, 2, 3, 4, 5, 10, 15, 20, or ranges including and/or spanning the aforementioned values. In several embodiments, k is 2. In several embodiments, v is 2. In several embodiments, n is 4. In several embodiments, n is 43 or 44. In several embodiments, q is 3. In several embodiments, R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—. In some embodiments, Y′ is a random copolymer or block copolymer of W¹ and W² having p repeat units of W¹ and r repeat units of W².

In several embodiments, as shown elsewhere herein, the targeting portion comprises one or more pendant liver targeting moieties decorating a portion of the linker. In several embodiments, the portion of the linker is a polymeric chain with pendant targeting agents attached randomly or in blocks along the chain. In some embodiments, the polymeric chain comprises an acrylate portion (e.g., acrylate polymers and/or acrylate copolymers). In several embodiments, the acrylate portion comprises an acrylate unit comprising a pendant liver targeting agent. In several embodiments, the acrylate portion further comprises an acrylate unit not comprising a pendant liver targeting agent.

In some embodiments, Y is a linker resulting from one or more reactions involving at least one of the following: N-hydroxysuccinamidyl (NHS) linker, NHS ester linker, PEG linker, maleimide linker, vinylsulfone linker, pyridyl di-thiol-poly(ethylene glycol) linker, pyridyl di-thiol linker, n-nitrophenyl carbonate linker, or a nitrophenoxy poly(ethylene glycol)ester linker. The linker may have one or more mannose and/or a mannose receptor-targeting moieties (including, but not limited to, one or more of mannosamine, N-acetylmannosamine, or N-acetylglucosamine) bound to it. In embodiments, Y comprises an antibody, an antibody fragment, a peptide, or a disulfanyl ethyl ester to which one or more mannose and/or mannose receptor-targeting moieties (including, but not limited to, one or more of mannosamine, N-acetylmannosamine, or N-acetylglucosamine) are bound.

In some embodiments, —[Y(—Z)ₚ]— comprises one of the following structures:

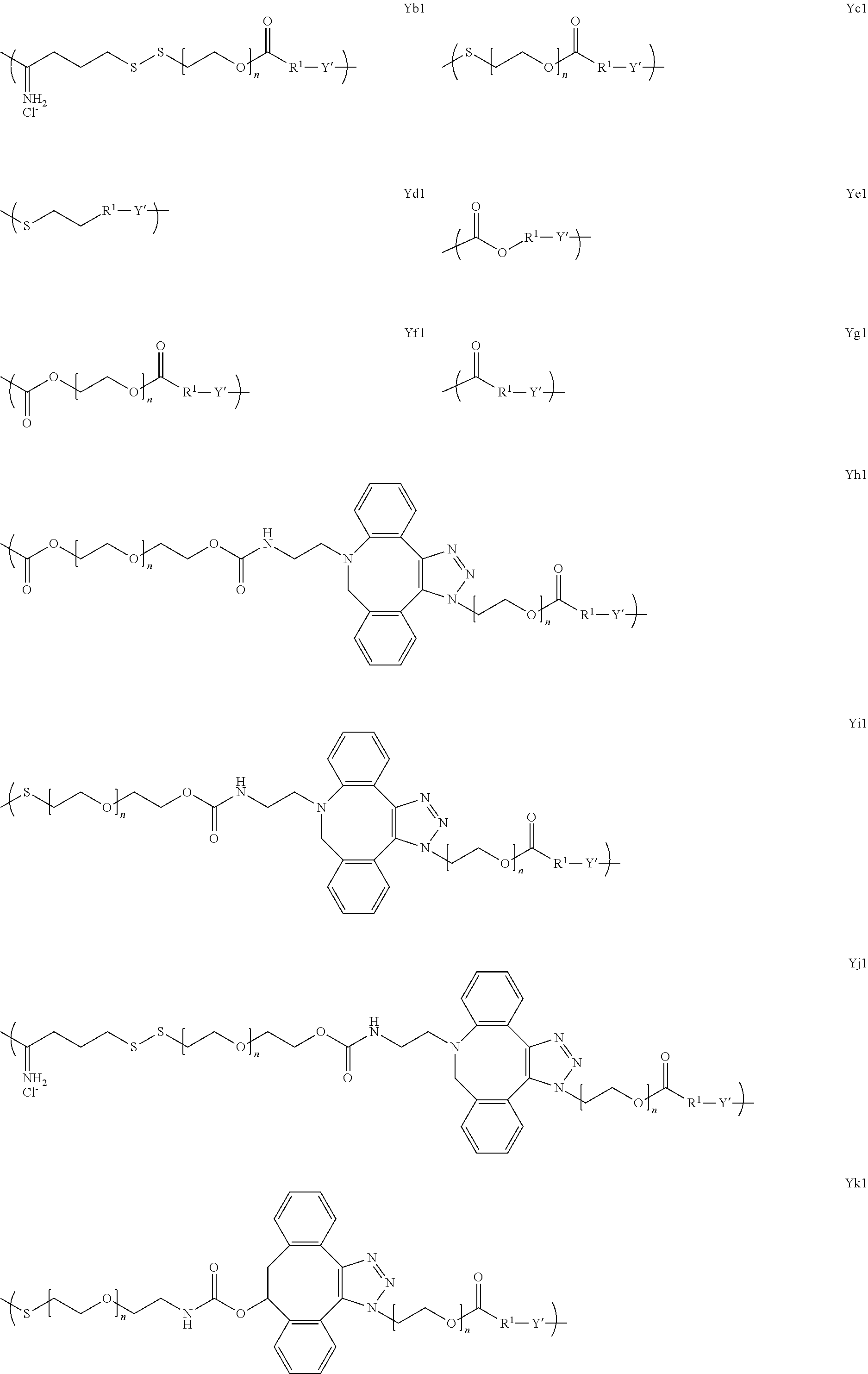

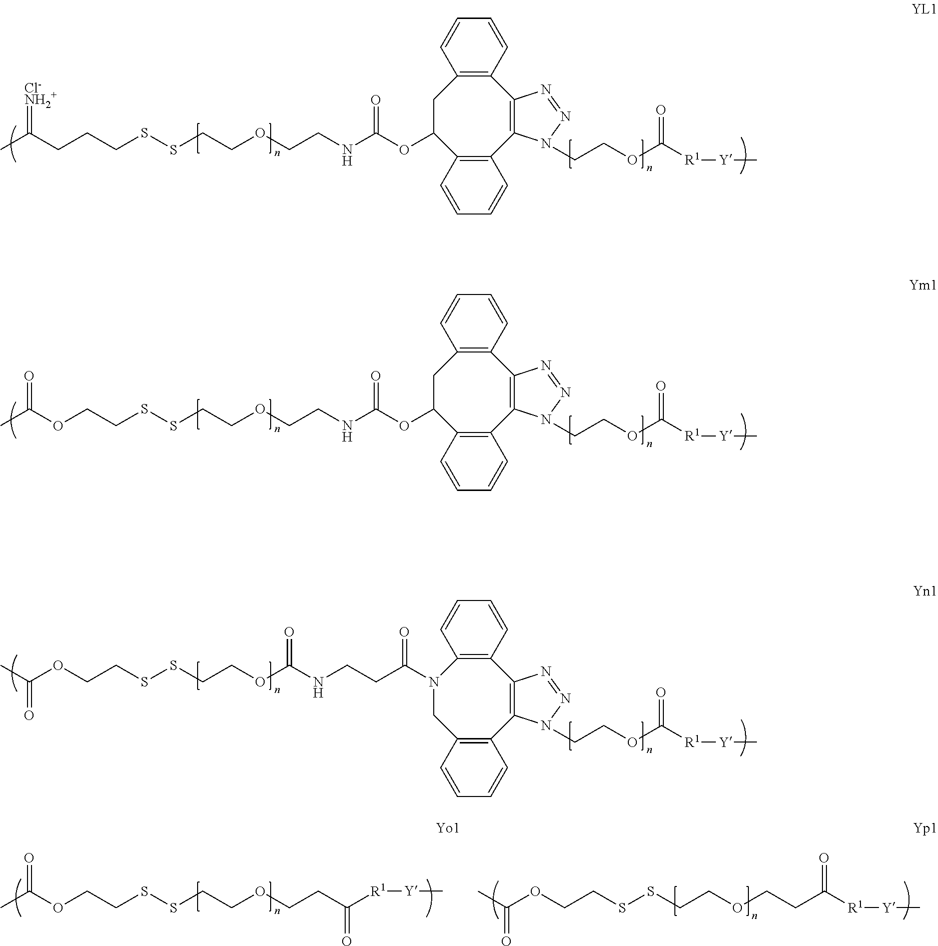

where the variables are as disclosed elsewhere herein.

In some embodiments, other linker structures can be found in U.S. Application Publication Nos. U.S. 2017/0007708A1 and 2016/0243248A1 and International Publication No. WO 2017/046652, each of which is incorporated by reference in its entirety.

In several embodiments, various ratios of W¹ to W² are used (e.g., W¹ and W² as provided in any of the Formulae disclosed elsewhere herein). In some embodiments, a majority of Y′ repeat units comprise W¹. In some embodiments, the ratio of W¹ to W² is equal to or greater than about 50:1, about 25:1, about 10:1, about 5:1, about 4:1, about 2:1, about 1:1, about 1:2, about 1:4, about 1:5, about 1:10, about 1:25, about 1:50, and any ratio in between those listed, including endpoints. In some embodiments, the ratio of p to r is equal to or greater than about 50:1, about 25:1, about 10:1, about 5:1, about 4:1, about 2:1, about 1:1, about 1:2, about 1:4, about 1:5, about 1:10, about 1:25, about 1:50, and any ratio in between those listed, including endpoints. In some embodiments, a homopolymer of W¹ is provided without a W² portion.

X may be a foreign transplant antigen, or alloantigen, or autoimmune antigen. In some aspects, X represents an antigen against which a patient may develop or has developed an unwanted immune response. In some embodiments, X is an antigen against which a subject, such as a transplant recipient or autoimmune patient, develops an unwanted immune response. In several embodiments, X is a foreign extracellular vesicle, cell fragment, or cell containing alloantigens against which transplant recipients or autoimmune patients develop and unwanted immune response. In still further embodiments, X is a foreign food, animal, plant or environmental antigen against which patients develop an unwanted immune response. In certain aspects, X is a foreign therapeutic agent against which patients develop an unwanted immune response. In a further aspect, X is a synthetic self-antigen to which patients develop an unwanted immune response. In several embodiments, X is a tolerogenic portion of a larger antigen. In certain embodiments, X, or a portion of X, is, is at least, or is at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids in length (or any range derivable therein). In some embodiments, X is an asparaginase antigen or an ovalbumin antigen. In several embodiments, X is an immunogenic fragment of one or more of myelin basic protein, myelin oligodendrocyte glycoprotein, proteolipid protein, insulin, proinsulin, preproinsulin, high molecular weight glutenin, low molecular weight glutenin, alpha- or gamma-gliadin, hordein, secalin, or avenin.

In some embodiments, Z, the moiety that specifically targets a mannose receptor, is selected from the group consisting of α-linked mannose, β-linked mannose, substituted mannose, mannose-6-phosphate, N-acetyl mannosamine, and mannan having β(1-4), α(1-6), α(1-2), and/or α(1-3) linkages. In some aspects, the mannose receptor is the mannose-6-phosphate receptor.

Some aspects of the disclosure are directed towards the use of a composition as disclosed herein in any method disclosed herein. Some embodiment provide for the use of any composition disclosed herein for the induction of tolerance to the antigen or a tolerogenic portion thereof. It is specifically contemplated that any step or element of an embodiments may be implemented in the context of any other step(s) or element(s) of a different embodiment disclosed herein.

In some embodiments, a method of inducing immunological tolerance to an antigen target is provided. The method comprises administering a composition of Formula 1:

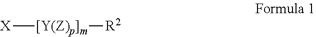

where the variables are as disclosed elsewhere herein. In some embodiments, for example, X comprises an antigen or a tolerogenic portion thereof, Y comprises a linker moiety, Z comprises a moiety that specifically targets a mannose receptor, p is an integer from 2 to 250, m is an integer from 1 to 100, R² is any of functional groups I-III:

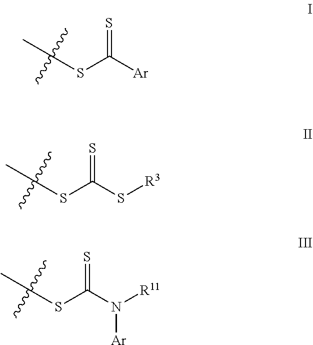

where Ar is a substituted or unsubstituted aromatic group, R³ is any carbon-containing linear or heterocyclic moiety, and R¹¹ is hydrogen or an alkyl group. In some embodiments, R² is one of the functional groups:

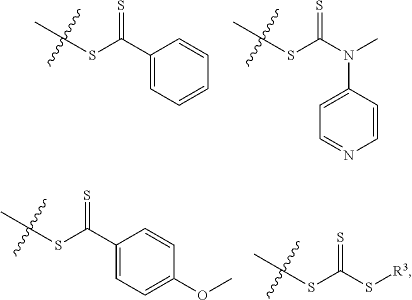

where R³ is as defined above.

In some embodiments, Y is a linker as described elsewhere herein. In several embodiments, Y is a linker resulting from one or more reactions involving at least one of the following N-hydroxysuccinamidyl (NHS) linker, NHS ester linker, PEG linker, maleimide linker, vinylsulfone linker, pyridyl di-thiol-poly(ethylene glycol) linker, pyridyl di-thiol linker, n-nitrophenyl carbonate linker, or a nitrophenoxy poly(ethylene glycol)ester linker. The linker may have one or more mannose moieties or mannose receptor-targeting moieties bound to it. In embodiments, Y comprises an antibody, an antibody fragment, a peptide, or a disulfanyl ethyl ester to which one or more mannose moieties or mannose receptor-targeting moieties are bound. In several embodiments, —[Y(—Z)ₚ]— is as disclosed elsewhere herein. For example, in some aspects, —[Y(Z)ₚ]— is a group represented by one of sequence Formula Ya to Yr:

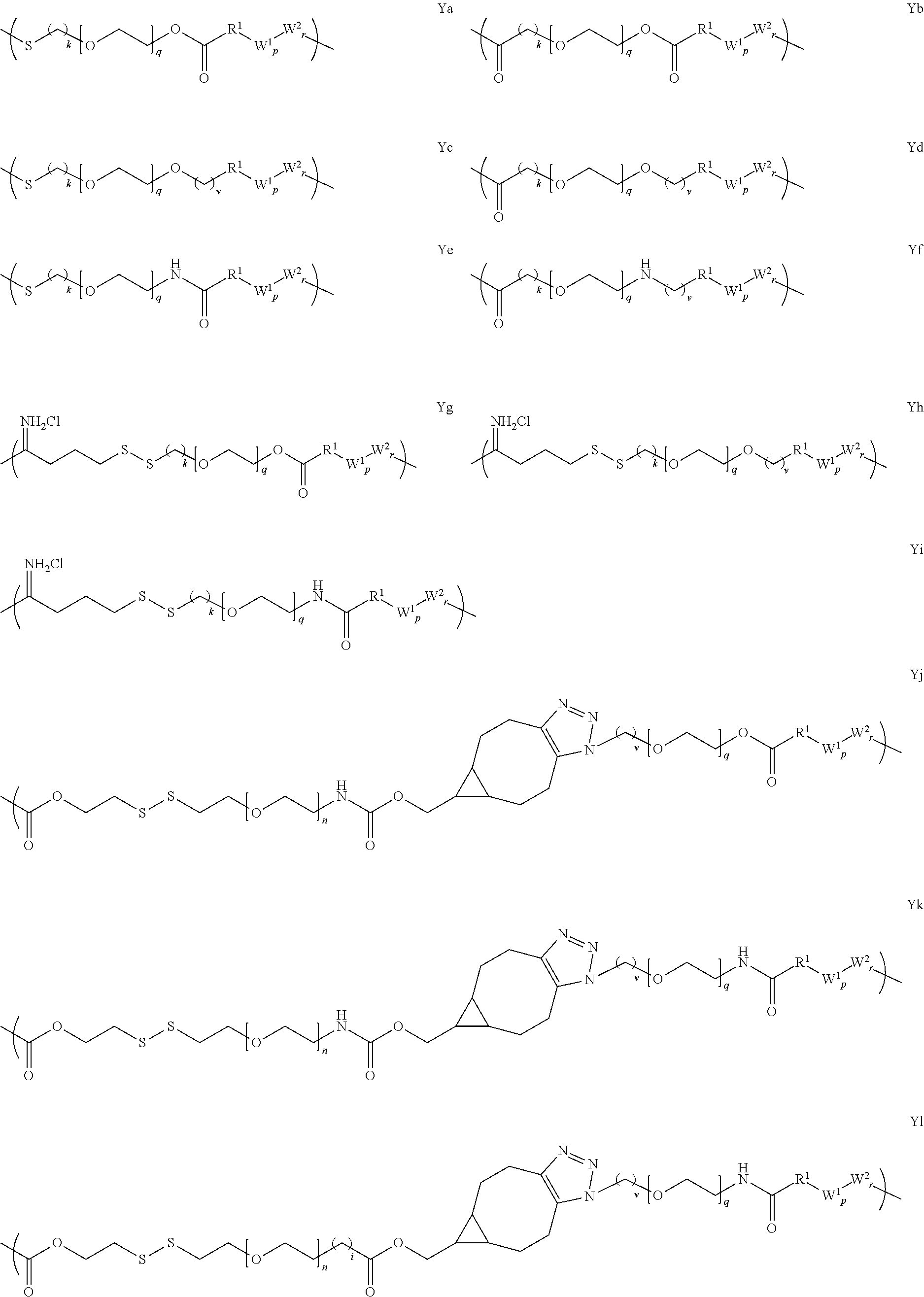

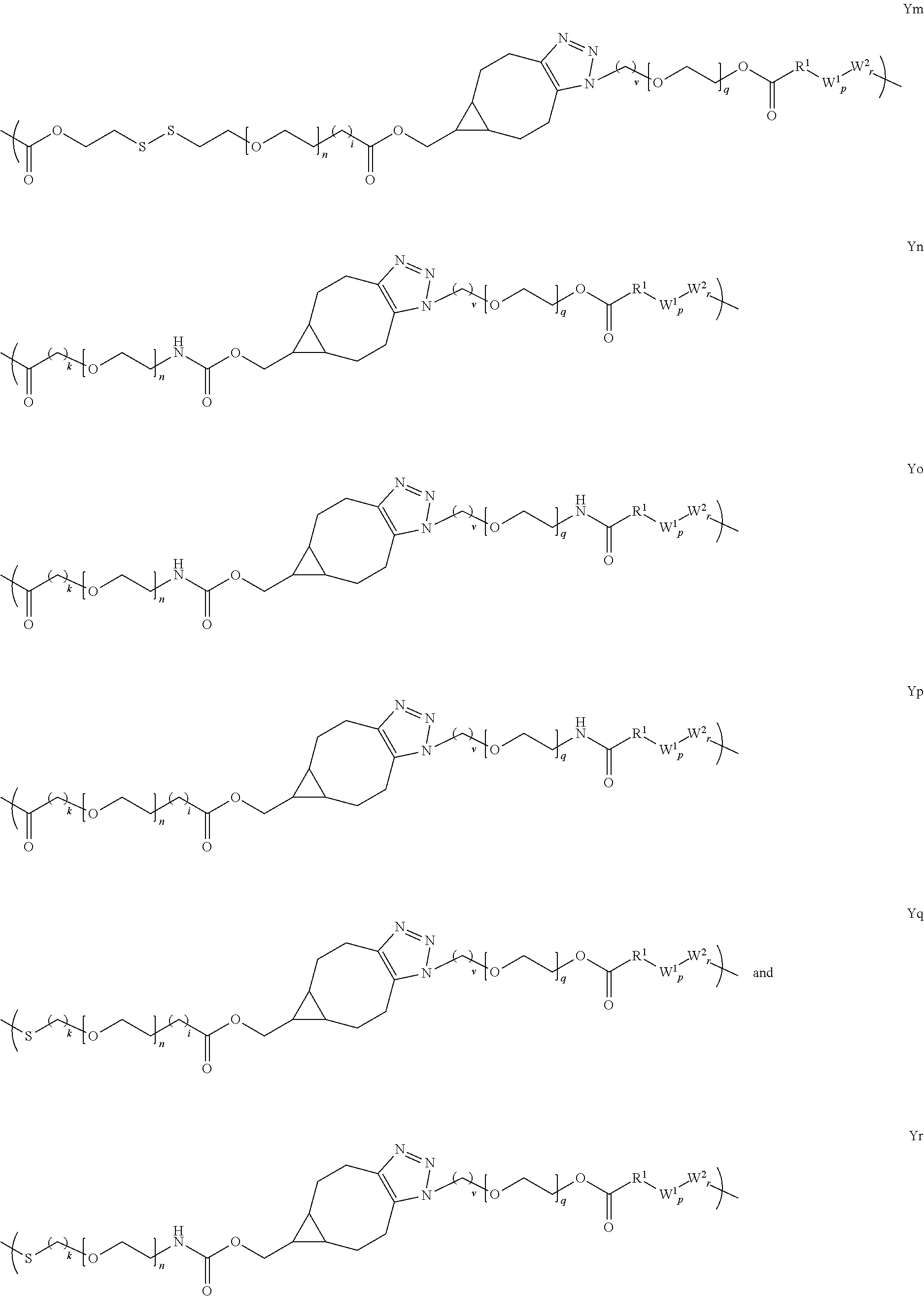

where the left, opening parentheses “(” signifies the location of the bond between X and Y, the right, closing parentheses “)” signifies the location of the bond between Y and R², n is an integer from 1 to 100, q is an integer from 1 to 44, k is an integer from 1 to 12, i is an integer from 0 to 20, v is an integer from 1 to 4, R₁ is —CH₂—, —(CH₂)—C(CH₃)(CN)—, —(CH₂)₂—C(CH₃)(CH₃)—, —(CH₂)₂—CH(CH₃)— or —CH(CH₃)—, W¹ and W² are as depicted below:

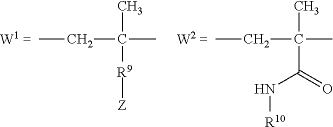

where Z is mannose or a mannose receptor-targeting moiety, R⁹ is a direct bond, —(CH₂)₂—NH—C(O)— (an ethylacetamido group or “EtAcN”) or —(CH₂)₂—(O—CH₂—CH₂)ₜ—NH—C(O)— (a pegylated ethylacetamido group or “Et-PEGₜ-AcN”), t is an integer from 1 to 5, 1 to 3, or 1 or 2, p is an integer from 2 to 250, R¹⁰ is an aliphatic group, an alcohol, an aliphatic amine-containing group, or an aliphatic alcohol, and r is an integer from 0 to 250. In some embodiments, R¹⁰ is a C_{f}alkyl or C_{f}alkylOH_{g}, where f represents the number of carbons in the alkyl group and is an integer between 0 and 10, and g represents the number of hydroxyl groups present on the alkyl group and is an integer between 0 and 10. In some embodiments, R¹⁰ is 2-hydroxyethyl. In some aspects —W¹ₚW²_{q}— represents a block copolymer or a random copolymer of W¹ and W² monomers.

X may be a foreign transplant antigen, or alloantigen, or autoimmune antigen. In some aspects, X represents an antigen against which a patient may develop or has developed an unwanted immune response. In some embodiments, X can be an antigen against which a subject, such as a transplant recipient or autoimmune patient, develops an unwanted immune response. In several embodiments, X can be a foreign extracellular vesicle, cell fragment, or cell containing alloantigens against which transplant recipients or autoimmune patients develop and unwanted immune response. In still further embodiments, X can be a foreign food, animal, plant or environmental antigen against which patients develop an unwanted immune response. In certain aspects X can be a foreign therapeutic agent against which patients develop an unwanted immune response. In a further aspect X can be a synthetic self-antigen to which patients develop an unwanted immune response. In several embodiments, X can be a tolerogenic portion of a larger antigen. In certain embodiments, X, or a portion of X, is at least, or is at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids in length (or any range derivable therein). In some embodiments, X is an asparaginase antigen or an ovalbumin antigen. In specific embodiments, X is an asparaginase antigen or an ovalbumin antigen, Z, the moiety that specifically targets a mannose receptor, may be selected from the group consisting of α-linked mannose, β-linked mannose, substituted mannose, mannose-6-phosphate, N-acetyl mannosamine, and mannan having β(1-4), α(1-6), α(1-2), and/or α(1-3) linkages.

In some aspects, a method of treating or preventing an unwanted immune response against an antigen is provided. The method comprises administering to a subject in need of suppression of an immune response to the antigen an effective amount of a composition comprising a compound of Formula 1. The composition can be administered for clearance of a circulating protein or peptide or antibody that specifically binds to antigen moiety X, which circulating protein or peptide or antibody is causatively involved in transplant rejection, immune response against a therapeutic agent, autoimmune disease, hypersensitivity and/or allergy. The composition can be administered in an amount effective to reduce a concentration of the antibodies that are causatively involved in transplant rejection, immune response against a therapeutic agent, autoimmune disease, hypersensitivity and/or allergy in blood of the patient by at least 50% w/w, as measured at a time between about 12 to about 48 hours after the administration. The composition can administered for tolerization of a patient with respect to antigen moiety X.

Other embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “effective amount” or “therapeutically effective amount” refer to that amount of a composition of the disclosure that is sufficient to effect treatment, as defined herein, when administered to a mammal in need of such treatment. This amount will vary depending upon the subject and disease condition being treated, the weight and age of the subject, the severity of the disease condition, the particular composition of the disclosure chosen, the dosing regimen to be followed, timing of administration, manner of administration and the like, all of which can readily be determined by one of ordinary skill in the art.

The “numerical values” and “ranges” provided for the various substituents are intended to encompass all integers within the recited range. For example, when defining n as an integer representing a mixture including from 1 to 100, where the mixture typically encompasses the integer specified as n±10% (or for smaller integers from 1 to about 25, ±3), it should be understood that n can be an integer from 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, or any between those listed) The terms “±10%” or “±3” should be understood to disclose and provide specific support for equivalent ranges wherever used.

The term “optional” or “optionally” means that the subsequently described event or circumstance may or may not occur, and that the description includes instances where said event or circumstance occurs and instances in which it does not.

A peptide, protein, or fragment that specifically binds a particular target is referred to as a “ligand” for that target.

A “polypeptide” is a term that refers to a chain of amino acid residues, regardless of post-translational modification (e.g., phosphorylation or glycosylation) and/or complexation with additional polypeptides, and/or synthesis into multisubunit complexes with nucleic acids and/or carbohydrates, or other molecules. Proteoglycans therefore also are referred to herein as polypeptides. A long polypeptide (having over 50 amino acids) is referred to as a “protein.” A short polypeptide (having 50 amino acids or fewer) is referred to as a “peptide.” Depending upon size, amino acid composition and three dimensional structure, certain polypeptides can be referred to as an “antigen-binding molecule,” “antibody,” an “antibody fragment” or a “ligand.” Polypeptides can be produced by a number of methods, many of which are well known in the art. For example, polypeptides can be obtained by extraction (e.g., from isolated cells), by expression of a recombinant nucleic acid encoding the polypeptide, or by chemical synthesis. Polypeptides can be produced by, for example, recombinant technology, and expression vectors encoding the polypeptide introduced into host cells (e.g., by transformation or transfection) for expression of the encoded polypeptide.

As used herein, “pharmaceutically acceptable carrier” or “pharmaceutically acceptable excipient” includes any and all solvents, dispersion media, coatings, antibacterial and antifungal agents, isotonic and absorption delaying agents and the like. In several embodiments, these media and agents can be used in combination with pharmaceutically active substances. Except insofar as any conventional media or agent is incompatible with the active ingredient, its use in the therapeutic compositions is contemplated. Supplementary active ingredients can also be incorporated into the compositions.

The term “purified” as used herein with reference to a polypeptide refers to a polypeptide that has been chemically or biologically synthesized and is thus substantially uncontaminated by other polypeptides, or has been separated or isolated from most other cellular components by which it is naturally accompanied (e.g., other cellular proteins, nucleic acids, or cellular components such as lipid membrane). An example of a purified polypeptide is one that is at least 70%, by dry weight, free from the proteins and naturally occurring organic molecules with which it naturally associates. A preparation of a purified polypeptide therefore can be, for example, at least 80%, at least 90%, or at least 99%, by dry weight, the polypeptide. Polypeptides also can be engineered to contain a tag sequence (e.g., a polyhistidine tag, a myc tag, a FLAG® tag, a SNAP® tag, or other affinity tag) that facilitates purification or marking (e.g., capture onto an affinity matrix, visualization under a microscope). Thus a purified composition that comprises a polypeptide refers to a purified polypeptide unless otherwise indicated. The term “isolated” indicates that the polypeptides or nucleic acids of the disclosure are not in their natural environment. Isolated products of the disclosure can thus be contained in a culture supernatant, partially enriched, produced from heterologous sources, cloned in a vector or formulated with a vehicle, etc.

The term “sequence identity” is used with regard to polypeptide or polynucleotide sequence comparisons. This expression in particular refers to a percentage of sequence identity, for example at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Particularly, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide. In several embodiments, despite differing sequences from a reference nucleotide (or corresponding polypeptide) a polynucleotide exhibits at least some degree of functional equivalence to the reference sequence, and in some embodiments, enhanced function.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The term “unwanted immune response” refers to a reaction by the immune system of a subject, which in the given situation is not desirable. The reaction of the immune system is unwanted if such reaction does not lead to the prevention, reduction, or healing of a disease or disorder but instead causes, enhances or worsens a disorder or disease. Typically, a reaction of the immune system causes, enhances or worsens a disease if it is directed against an inappropriate target. Exemplified, an unwanted immune response includes but is not limited to transplant rejection, immune response against a therapeutic agent, autoimmune disease, and allergy or hypersensitivity.

The term “operatively linked” refers to a situation where two components are combined to form the active complex prior to binding at the target site. For example, a molecule conjugated to one-half of a biotin-streptavidin complex and an antigen complexed to the other one-half of the biotin-streptavidin complex are operatively linked through complexation of the biotin and streptavidin molecules. The term operatively linked is also intended to refer to covalent or chemical linkages that conjugate two molecules together.

Throughout this application, the term “about” is used according to its plain and ordinary meaning in the area of protein chemistry to indicate that a value includes the standard deviation of error for the device or method being employed to determine the value.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Methods may involve multiple administrations of one or more compounds, compositions, and/or agents. In certain embodiments, cells or a subject are provided with a tolerance inducing agent prior to administering the composition for which a tolerance is being induced. It is contemplated that compounds, compositions, and/or agents may be formulated in a pharmaceutically acceptable formulation in certain embodiments of the invention.

A “disease” is defined as a pathological condition of a body part, an organ, or a system resulting from any cause, such as infection, genetic defect, or environmental stress. A “health-related condition” is defined herein to refer to a condition of a body part, an organ, or a system that may not be pathological, but for which treatment is sought. Examples include conditions for which cosmetic therapy is sought, such as skin wrinkling, skin blemishes, and the like. The disease can be any disease, and non-limiting examples include hyperproliferative diseases such as cancer and premalignant lesions, wounds, and infections.

A subject may be “predicted to have” a disease if the subject exhibits a characteristic, condition, or behavior that increases the likelihood of getting a disease. The characteristic, condition, or behavior that increases the likelihood of getting a disease is known as a risk factor, and may be behavioral, physiological, demographic, environmental, or genetic in nature. Behavioral risk factors usually relate to actions that a subject has chosen to take. Demographic risk factors are those that relate to the overall population, such as age or gender. Environmental risk factors include those that are related to exposure to objects in an environment, such as air pollution and access to clean water. Genetic risk factors are based on an individual's genetic makeup, and may reflect interaction between the genes of the individual and environmental factors. A subject having a high number of manifestations of risk factors associated with a disease may be at increased risk of developing the disease, and may be “predicted to have” the disease.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

As used herein, an “antigen” is any substance that serves as a target for the receptors of an adaptive immune response, such as the T cell receptor, major histocompatibility complex class I and II, B cell receptor or an antibody. In some embodiments, an antigen may originate from within the body (e.g., “self,” “auto” or “endogenous”). In additional embodiments, an antigen may originate from outside the body (“non-self,” “foreign” or “exogenous”), having entered, for example, by inhalation, ingestion, injection, or transplantation, transdermal, etc. In some embodiments, an exogenous antigen may be biochemically modified in the body. Foreign antigens include, but are not limited to, food antigens, animal antigens, plant antigens, environmental antigens, therapeutic agents, as well as antigens present in an allograft transplant. Non-limiting examples of antigens are provided herein.

An “antigen-binding molecule” as used herein relates to molecules, in particular to proteins such as immunoglobulin molecules, which contain antibody variable regions providing a binding (specific binding in some embodiments) to an epitope. The antibody variable region can be present in, for example, a complete antibody, an antibody fragment, and a recombinant derivative of an antibody or antibody fragment. The term “antigen-binding fragment” of an antibody (or “binding portion”), as used herein, refers to one or more fragments of an antibody that retain the ability to specifically bind a target sequence. Antigen-binding fragments containing antibody variable regions include (without limitation) “Fv”, “Fab”, and “F(ab′)₂” regions, “single domain antibodies (sdAb)”, “nanobodies”, “single chain Fv (scFv)” fragments, “tandem scFvs” (V_{H}A-V_{L}A-V_{H}B-V_{L}B), “diabodies”, “triabodies” or “tribodies”, “single-chain diabodies (scDb)”, and “bi-specific T-cell engagers (BiTEs)”.

An “epitope”, also known as antigenic determinant, is the segment of a macromolecule, e.g. a protein, which is recognized by the adaptive immune system, such as by antibodies, B cells, major histocompatibility complex molecules, or T cells. An epitope is that part or segment of a macromolecule capable of binding to an antibody or antigen-binding fragment thereof. In this context, the term “binding” in particular relates to a specific binding. In the context of several embodiments of the present invention it is preferred that the term “epitope” refers to the segment of protein or polyprotein that is recognized by the immune system.

The term mannose or mannosylating moiety refers to a monosaccharide sugar that exists both in open-chain form and in cyclic form, having D- and L-isomers. In the cyclic form, there are two anomers, namely alpha and beta. In the alpha form, the C1 alcohol group is in the axial position, whereas in the beta form, the C1 alcohol group is in the equatorial position. In particular, “mannose” refers to the cyclic six-membered pyranose, more in particular the D-isomer and even more particularly the alpha-D-form (α-D-mannosee). The structure and numbering of mannose on non-limiting examples of stereochemical illustration. In the current formulation, the mannose residue is a single sugar that is connected to the backbone of the polymer via a single site such as the primary alcohols that attach to C1-C4 and C6. Thus, one of the alcohols is used to connect the mannose to the polymer while the other alcohols remain OH groups at neutral pH. In several embodiments, the advantageous polyfunctionality of the approaches disclosed herein comes, at least in part, from the multiple monomers that are used to decorate the side of the polymer.

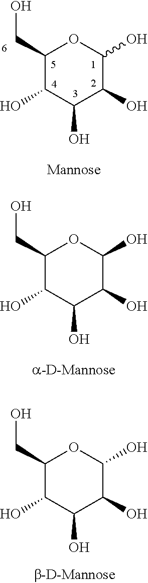 [SMILES: OC[C@H]1OC(O)[C@@H](O)[C@@H](O)[C@@H]1O ; OC[C@H]1O[C@@H](O)[C@@H](O)[C@@H](O)[C@@H]1O ; OC[C@H]1O[C@H](O)[C@@H](O)[C@@H](O)[C@@H]1O]

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from C₁₋₆ alkyl, C₁₋₆ alkenyl, C₁₋₆ alkynyl, C₁₋₆ cycloalkyl, C₁₋₆ aryl, C₁₋₆ heteroaryl, C₁₋₆ heterocyclyl, C₁₋₆ alkoxy, C₁₋₆ acyl, cyano, hydroxyl, an amino, halogen substituted C₁₋₆alkyl, halogen substituted C₁₋₆alkoxy, and halogen.

As used herein, a “chemical modification” refers to a change in the naturally-occurring chemical structure of one or more amino acids of a polypeptide. Such modifications can be made to a side chain or a terminus, e.g., changing the amino-terminus or carboxyl terminus. In some embodiments, the modifications are useful for creating chemical groups that can conveniently be used to link the polypeptides to other materials, or to attach a therapeutic agent.

“Conservative changes” can generally be made to an amino acid sequence without altering activity. These changes are termed “conservative substitutions” or mutations; that is, an amino acid belonging to a grouping of amino acids having a particular size or characteristic can be substituted for another amino acid. Substitutes for an amino acid sequence can be selected from other members of the class to which the amino acid belongs. For example, the nonpolar (hydrophobic) amino acids include alanine, leucine, isoleucine, valine, proline, phenylalanine, tryptophan, methionine, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such substitutions are not expected to substantially affect apparent molecular weight as determined by polyacrylamide gel electrophoresis or isoelectric point. Conservative substitutions also include substituting optical isomers of the sequences for other optical isomers, specifically d amino acids for 1 amino acids for one or more residues of a sequence. Moreover, all of the amino acids in a sequence can undergo a d to 1 isomer substitution. Exemplary conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free —NH₂. Yet another type of conservative substitution constitutes the case where amino acids with desired chemical relativities are introduced to impart reactive sites for chemical conjugation reactions, if the need for chemical derivatization arises. Such amino acids include but are not limited to Cys (to insert a sulfhydryl group), Lys (to insert a primary amine), Asp and Glu (to insert a carboxylic acid group), or specialized noncanonical amino acids containing ketone, azide, alkyne, alkene, and tetrazine side-chains. Conservative substitutions or additions of free —NH₂ or —SH bearing amino acids can be particularly advantageous for chemical conjugation with the linkers and mannosylating moieties of Formula 1. Moreover, point mutations, deletions, and insertions of the polypeptide sequences or corresponding nucleic acid sequences can in some cases be made without a loss of function of the polypeptide or nucleic acid fragment. Substitutions can include, e.g., 1, 2, 3, 4, 5, 6, 7, 8, 9, 10, 15, 20, 25, 30, 35, 40, 45, 50 or more residues (including any number of substitutions between those listed). A variant usable in embodiments herein may exhibit a total number of up to 200 (e.g., up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200, including any number in between those listed) changes in the amino acid sequence (e.g., exchanges, insertions, deletions, N-terminal truncations, and/or C-terminal truncations). In several embodiments, the number of changes is greater than 200. Additionally, in several embodiments, the variants include polypeptide sequences or corresponding nucleic acid sequences that exhibit a degree of functional equivalence with a reference (e.g., unmodified or native sequence). In several embodiments, the variants exhibit about 80%, about 85%, about 90%, about 95%, about 97%, about 98%, about 99% functional equivalence to an unmodified or native reference sequence (and any degree of functional equivalence between those listed). The amino acid residues described herein employ either the single letter amino acid designator or the three-letter abbreviation in keeping with the standard polypeptide nomenclature, J. Biol. Chem., (1969), 243, 3552-3559. All amino acid residue sequences are represented herein by formulae with left and right orientation in the conventional direction of amino-terminus to carboxy-terminus.

The term “liver-targeting moiety” refers to mannose moieties having the ability to direct, e.g., a polypeptide, to the cells of a liver expressing mannose receptors. The liver comprises different cell types, including but not limited to hepatocytes, sinusoidal epithelial cells, Kupffer cells, stellate cells, and/or dendritic cells. Typically, a liver-targeting moiety directs a polypeptide to one or more of these cells. On the surface of the respective liver cells, receptors are present which recognize and specifically bind the liver-targeting moiety. Liver-targeting can be achieved by chemical conjugation of an antigen or ligand to a mannosylating or mannansylating moiety. Naturally occurring desialylated proteins are not encompassed within the scope of certain embodiments of the present disclosure.

The term “random copolymer” refers to the product of simultaneous polymerization of two or more monomers in admixture, where the probability of finding a given monomeric unit at any given site in a polymer chain is independent of the nature of the neighboring units at that position (Bemoullian distribution). Thus, when the variable group identified as Wp represents a random copolymer, the chain can comprise any sequence from 2 up to about 150 W1 and W2 groups, such as: —W1-W2-W1-W2-; —W2-W1-W2-W1-; —W1-W1-W1-W2-; —W1-W1-W2-W2-; —W1-W2-W2-W1-; —W1-W2-W1-W2-W2-W1-W2-W1-; —W1-W1-W2-W2-W1-W2-W2-W1-; and W2-W2-W1-W2-W1-W1-W1-W2-W2-W1-W2-W2-W1; ad infinitum, where Z attached to the various W1 groups and the W1 and W2 groups themselves can be the same or different.

The term “sequence identity” is used with regard to polypeptide (or nucleic acid) sequence comparisons. This expression in particular refers to a percentage of sequence identity, for example at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Particularly, the polypeptide in question and the reference polypeptide exhibit the indicated sequence identity over a continuous stretch of 20, 30, 40, 45, 50, 60, 70, 80, 90, 100 or more amino acids (or any range derivable therein) or over the entire length of the reference polypeptide.

“Specific binding,” as that term is commonly used in the biological arts, refers to a molecule that binds to a target with a relatively high affinity as compared to non-target tissues, and generally involves a plurality of non-covalent interactions, such as electrostatic interactions, van der Waals interactions, hydrogen bonding, and the like. Specific binding interactions characterize antibody-antigen binding, enzyme-substrate binding, and certain protein-receptor interactions; while such molecules might bind tissues besides their specific targets from time to time, to the extent that such non-target binding is inconsequential, the high-affinity binding pair can still fall within the definition of specific binding.

The term “unwanted immune response” refers to a reaction by the immune system of a subject, which in the given situation is not desirable. The reaction of the immune system is unwanted if such reaction does not lead to the prevention, reduction, or healing of a disease or disorder but instead causes, enhances or worsens, or is otherwise associated with induction or worsening of a disorder or disease. Typically, a reaction of the immune system causes, enhances or worsens a disease if it is directed against an inappropriate target. Exemplified, an unwanted immune response includes but is not limited to transplant rejection, immune response against a therapeutic agent, autoimmune disease, and allergy or hypersensitivity.

The term “variant” is to be understood as a protein (or nucleic acid) which differs in comparison to the protein from which it is derived by one or more changes in its length, sequence, or structure. The polypeptide from which a protein variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence or structure in comparison to the parent molecule. Also encompassed are modified molecules such as but not limited to post-translationally modified proteins (e.g. glycosylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Naturally occurring and artificially constructed variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, e.g., is functionally active. A variant can be characterized by a certain degree of sequence identity to the parent polypeptide from which it is derived. More precisely, a protein variant in the context of the present disclosure may exhibit at least 80% sequence identity to its parent polypeptide. Preferably, the sequence identity of protein variants is over a continuous stretch of 20, 30, 40, 45, 50, 60, 70, 80, 90, 100 or more amino acids. As discussed above, in several embodiments variants exhibit about 80%, about 85%, about 90%, about 95%, about 97%, about 98%, about 99% functional equivalence to an unmodified or native reference sequence (and any degree of functional equivalence between those listed).

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-1G. OVA-p(Man) induces T cell deletion. BLK6 mice were treated with saline, or 10 μg of OVA in the form of free OVA or OVA conjugated to p(Man) (OVA-p(Man)) one day and 7 days after an adoptive transfer of 7.0×105 OTI and OTII T cells. The mice were challenged with an intradermal injection of LSP and OVA on 14 days after the initial OTI and OTII T cell transfer, then the immune response in the draining lymph nodes (dLNs) was assessed on day 19 via flow cytometry. FIG. 1A Fraction of OTII T cells in the dLNs on day 19. FIG. 1B Fraction of OTI T cells in the dLNs on day 19. FIG. 1C Fraction of OTII T cells in the liver on day 19. FIG. 1D Fraction of OTI T cells in the liver on day 19. FIG. 1E Fraction of Tregs in the dLN on day 19. FIG. 1F Fraction of Tregs in the liver on day 19. FIG. 1G Fraction of T follicular helper cells (Tfh) as a fraction of total CD4 T cells in the dLNs on day 19.

FIGS. 2A-2B: OVA-p(Man) induces T cells Anergy in DLs. BLK6 mice were treated with saline, or 10 μg of OVA in the form of free OVA or OVA conjugated to p(Man) (OVA-p(Man)) one day and 7 days after an adoptive transfer of 7.0×105 OTI and OTII T cells. The mice were challenged with an intradermal injection of LPS and OVA on 14 days after the initial OTI and OTII T cell transfer, and immune response in the draining lymph nodes (dLNs) was assessed on day 19. Cells from the dLN were restimulated with OVA (A) or SIINFEKL (peptide) for 6 hours, then the percentage of IFN-γ producing cells was determined by flow cytometry. FIG. 2A Percentage of IFN-γ producing CD4+ T cells in the dLNs. FIG. 2B Percentage of IFN-γ producing CD8+ T cells in the dLNs.

FIGS. 3A-3E. Tolerance induction to protein therapeutics. Five BALB/c mice per group were injected with 2.5 μg of asparaginase formulated as free asparaginase (ASNase) or conjugated to p(Man) (ASNase-p(Man)) once a week for 3 weeks and then were switched to 15 μg of ASNase i.v. one a week for 8 weeks. During the initial 3 weeks ASNase-p(Man) was administered via either i.v. or subcutaneous injection. Sera was taken from the mice and monitored weekly for the presence of αASNase. FIG. 3A Pan IgG αASNase titers of mice treated with ASNase and ASNase-p(Man) via i.v. and subcutaneous injection. FIG. 3B αASNase IgG1 titers of treatment groups on day 38. FIG. 3C αASNase IgG2a titers of treatment groups on day 38. FIG. 3D αASNase IgG2b titers of treatment groups on day 38. FIG. 3E. αASNase IgG3 titers of treatment groups on day 38.

FIGS. 4A-4B. Response to p(Man) conjugates. FIG. 4A Bone marrow of animals treated with ASNase-p(Man) had fewer αASNase plasma cells than animals that were treated with ASNase. FIG. 4B Spleens of animals treated with ASNase-p(Man) had a greater percentage of IL-10 producing B regulatory cells.

FIG. 5. Tolerance induction to protein therapeutics. FIG. 5 is a graph depicting tolerance induction to the protein therapeutic asparaginase conjugated to p(Man) linker and mannose. Five BALB/c mice per group were injected with 2.5 μg of asparaginase formulated as free asparaginase (ASNase) or conjugated to p(Man) (ASNase-p(Man)) once a week for 3 weeks and then were switched to 15 μg of ASNase i.v. one a week for 7 weeks. Sera was taken from the mice and monitored weekly for the presence of anti-ASNase. Over the entire time of analysis none of the animals treated with ASNase-p(Man) developed measureable levels of antibodies against ASNase.

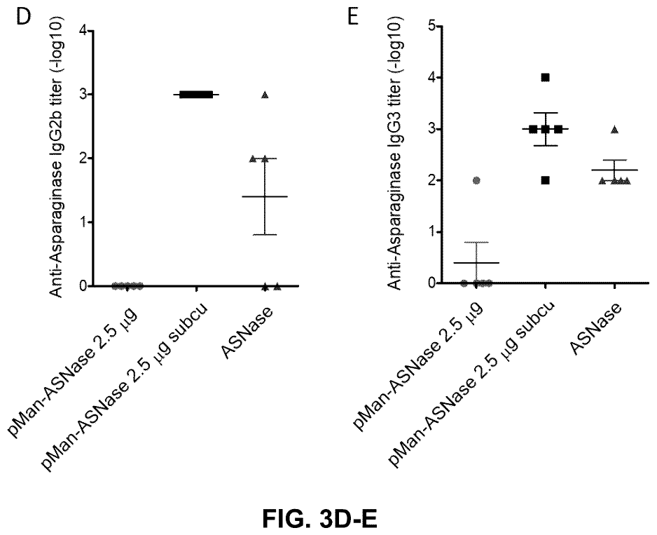

FIG. 6 p(Man)-ASNase administration regimen for assessment of p(Man)-protein conjugates on anti-asparaginase (anti-ASNase) humoral immune response.

FIGS. 7A-7B. Assessment of p(Man)-protein conjugates on anti-asparaginase (anti-ASNase) humoral immune response. FIG. 7A is a graph depicting serum asparagine concentration for days 71, 73, and 76. Mice treated with only saline or p(Man)-ASNase have a significantly lower serum asparagine concentration as compared to animals that had been treated with saline and then administered wt ASNase. FIG. 7B is a graph depicting serum asparagine concentration vs the anti-ASNase titer for each animal in the study. A strong correlation (r=0.8) between serum asparagine concentration and anti-ASNase titer is evident.

### DETAILED DESCRIPTION

Several embodiments disclosed herein overcome the deficiencies of the prior art by providing compositions comprising mannose-fused antigens. The compositions may be used to prevent immunity or reduce an immune response protein-based drugs that would otherwise elicit an immune response.

Multiple mannose binding receptors are expressed by antigen presenting cells (APCs) and serve as gateways for antigen uptake and antigen cross presentation by these cells to T cells. Antigens taken up by APCs and presented to T cells in the absence of co-stimulation leads to T cell deletion, inactivity, and the formation of T regulator cells, that control antigen specific immune responses.

The present disclosure provides, in several embodiments, certain therapeutic compositions that are targeted for delivery to (and for uptake by) antigen presenting cells, particularly hepatocytes, LSECs, Kupffer cells and/or stellate cells, more particularly hepatocytes and/or LSECs, and even more particularly to specifically bind mannose-binding receptors.

Liver targeting facilitates two mechanisms of treatment: tolerization and clearance. Tolerization takes advantage of the liver's role in clearing apoptotic cells and processing their proteins to be recognized by the immune system as “self,” as well as the liver's role in sampling peripheral proteins for immune tolerance. Clearance takes advantage of the liver's role in blood purification by rapidly removing and breaking down toxins, polypeptides and the like. Targeting of these compositions to the liver is accomplished by a mannosating moiety. The mannosylating moiety is chemically conjugated. The antigen can be endogenous (a self-antigen) or exogenous (a foreign antigen), including but not limited to: a foreign transplant antigen against which transplant recipients develop an unwanted immune response (e.g., transplant rejection), a foreign food, animal, plant or environmental antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease), or a tolerogenic portion (e.g., a fragment or an epitope) thereof; these compositions are useful for inducing tolerization to the antigen. Accordingly, the compositions of the present disclosure can be used for treating an unwanted immune response, e.g., transplant rejection, an immune response against a therapeutic agent, an autoimmune disease, and/or an allergy, depending on the embodiment. Also provided are pharmaceutical compositions containing a therapeutically effective amount of a composition of the disclosure admixed with at least one pharmaceutically acceptable excipient. In another aspect, the disclosure provides methods for the treatment of an unwanted immune response, such as transplant rejection, response against a therapeutic agent, autoimmune disease or allergy.

### B. Chemical Definitions

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

As used herein, the term “amino” means —NH2; the term “nitro” means —NO2; the term “halo” or “halogen” designates —F, —Cl, —Br or —I; the term “mercapto” means —SH; the term “cyano” means —CN; the term “azido” means —N3; the term “silyl” means —SiH3, and the term “hydroxy” means —OH. In certain embodiments, a halogen may be —Br or —I.

As used herein, a “monovalent anion” refers to anions of a −1 charge. Such anions are well-known to those of skill in the art. Non-limiting examples of monovalent anions include halides (e.g., F—, Cl—, Br— and I—), NO2-, NO3-, hydroxide (OH—) and azide (N3-).

As used herein, the structure indicates that the bond may be a single bond or a double bond. Those of skill in the chemical arts understand that in certain circumstances, a double bond between two particular atoms is chemically feasible and in certain circumstances, a double bond is not. The present invention therefore contemplates that a double bond may be formed only when chemically feasible.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C1-C10-alkyl has 1 to 10 carbon atoms. The groups, —CH3 (Me), —CH2CH3 (Et), —CH2CH2CH3 (n-Pr), —CH(CH3)2 (iso-Pr), —CH(CH2)2 (cyclopropyl), —CH2CH2CH2CH3 (n-Bu), —CH(CH3)CH2CH3 (sec-butyl), —CH2CH(CH3)2 (iso-butyl), —C(CH3)3 (tert-butyl), —CH2C(CH3)3 (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C1-C10-alkyl has 1 to 10 carbon atoms. The following groups are all non-limiting examples of heteroatom-substituted alkyl groups: trifluoromethyl, —CH2F, —CH2Cl, —CH2Br, —CH2OH, —CH2OCH3, —CH2OCH2CF3, —CH2OC(O)CH3, —CH2NH2, —CH2NHCH3, —CH2N(CH3)2, —CH2CH2Cl, —CH2CH2OH, CH2CH2OC(O)CH3, —CH2CH2NHCO2C(CH3)3, and —CH2Si(CH3)3.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

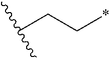 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group

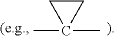

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cn-alkenyl, and heteroatom-substituted Cn-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cn-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C2-C10-alkenyl has 2 to 10 carbon atoms. Heteroatom-unsubstituted alkenyl groups include: —CH═CH2 (vinyl), —CH═CHCH3, —CH═CHCH2CH3, —CH2CH═CH2 (allyl), —CH2CH═CHCH3, and —CH═CH—C6H5. The term “heteroatom-substituted Cn-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C2-C10-alkenyl has 2 to 10 carbon atoms. The groups, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C6-C10-aryl has 6 to 10 carbon atoms. Non-limiting examples of heteroatom-unsubstituted aryl groups include phenyl (Ph), methylphenyl, (dimethyl)phenyl, —C6H4CH2CH3, —C6H4CH2CH2CH3, —C6H4CH(CH3)2, —C6H4CH(CH2)2, —C6H3(CH3)CH2CH3, —C6H4CH═CH2, —C6H4CH═CHCH3, —C6H4CECH, —C6H4C≡CCH3, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C1-C10-heteroaryl has 1 to 10 carbon atoms. Non-limiting examples of heteroatom-substituted aryl groups include the groups: —C6H4F, —C6H4Cl, —C6H4Br, —C6H4I, —C6H40H, —C6H40CH3, —C6H40CH2CH3, —C6H40C(O)CH3, —C6H4NH2, —C6H4NHCH3, —C6H4N(CH3)2, —C6H4CH2OH, —C6H4CH2OC(O)CH3, —C6H4CH2NH2, —C6H4CF3, —C6H4CN, —C6H4CHO, —C6H4CHO, —C6H4C(O)CH3, —C6H4C(O)C6H5, —C6H4CO2H, —C6H4CO2CH3, —C6H4CONH2, —C6H4CONHCH3, —C6H4CON(CH3)2, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents.

The term “aralkyl” includes heteroatom-unsubstituted aralkyl, heteroatom-substituted aralkyl, heteroatom-unsubstituted Cn-aralkyl, heteroatom-substituted Cn-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cn-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C7-C10-aralkyl has 7 to 10 carbon atoms. Non-limiting examples of heteroatom-unsubstituted aralkyls are: phenylmethyl (benzyl, Bn) and phenylethyl. The term “heteroatom-substituted Cn-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C2-C10-heteroaralkyl has 2 to 10 carbon atoms.

The term “acyl” includes straight-chain acyl, branched-chain acyl, cycloakyl, cyclic acyl, heteroatom-unsubstituted acyl, heteroatom-substituted acyl, heteroatom-unsubstituted Cn-acyl, heteroatom-substituted Cn-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cn-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C1-C10-acyl has 1 to 10 carbon atoms. The groups, —CHO, —C(O)CH3, —C(O)CH2CH3, —C(O)CH2CH2CH3, —C(O)CH(CH3)2, —C(O)CH(CH2)2, —C(O)C6H5, —C(O)C6H4CH3, —C(O)C6H4CH2CH3, and —COC6H3(CH3)2, are non-limiting examples of heteroatom-unsubstituted acyl groups. The term “heteroatom-substituted Cn-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C1-C10-acyl has 1 to 10 carbon atoms. The groups, —C(O)CH2CF3, —CO2H, —CO2-, —CO2CH3, —CO2CH2CH3, —CO2CH2CH2CH3, —CO2CH(CH3)2, —CO2CH(CH2)2, —C(O)NH2 (carbamoyl), —C(O)NHCH3, —C(O)NHCH2CH3, —CONHCH(CH3)2, —CONHCH(CH2)2, —CON(CH3)2, and —CONHCH2CF3, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cn-alkoxy, and heteroatom-substituted Cn-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cn-alkoxy” refers to a group, having the structure —OR, in which R is a heteroatom-unsubstituted Cn-alkyl, as that term is defined above. Heteroatom-unsubstituted alkoxy groups include: —OCH3, —OCH2CH3, —OCH2CH2CH3, —OCH(CH3)2, and —OCH(CH2)2. The term “heteroatom-substituted Cn-alkoxy” refers to a group, having the structure —OR, in which R is a heteroatom-substituted Cn-alkyl, as that term is defined above. For example, —OCH2CF3 is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cn-alkenyloxy, and heteroatom-substituted Cn-alkenyloxy. The term “heteroatom-unsubstituted Cn-alkenyloxy” refers to a group, having the structure —OR, in which R is a heteroatom-unsubstituted Cn-alkenyl, as that term is defined above. The term “heteroatom-substituted Cn-alkenyloxy” refers to a group, having the structure —OR, in which R is a heteroatom-substituted Cn-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cn-alkynyloxy, and heteroatom-substituted Cn-alkynyloxy. The term “heteroatom-unsubstituted Cn-alkynyloxy” refers to a group, having the structure —OR, in which R is a heteroatom-unsubstituted Cn-alkynyl, as that term is defined above. The term “heteroatom-substituted Cn-alkynyloxy” refers to a group, having the structure —OR, in which R is a heteroatom-substituted Cn-alkynyl, as that term is defined above.

The term “aryloxy” includes heteroatom-unsubstituted aryloxy, heteroatom-substituted aryloxy, heteroatom-unsubstituted Cn-aryloxy, heteroatom-substituted Cn-aryloxy, heteroaryloxy, and heterocyclic aryloxy groups. The term “heteroatom-unsubstituted Cn-aryloxy” refers to a group, having the structure —OAr, in which Ar is a heteroatom-unsubstituted Cn-aryl, as that term is defined above. A non-limiting example of a heteroatom-unsubstituted aryloxy group is —OC6H5. The term “heteroatom-substituted Cn-aryloxy” refers to a group, having the structure —OAr, in which Ar is a heteroatom-substituted Cn-aryl, as that term is defined above.

The term “aralkyloxy” includes heteroatom-unsubstituted aralkyloxy, heteroatom-substituted aralkyloxy, heteroatom-unsubstituted Cn-aralkyloxy, heteroatom-substituted Cn-aralkyloxy, heteroaralkyloxy, and heterocyclic aralkyloxy groups. The term “heteroatom-unsubstituted Cn-aralkyloxy” refers to a group, having the structure —OAr, in which Ar is a heteroatom-unsubstituted Cn-aralkyl, as that term is defined above. The term “heteroatom-substituted Cn-aralkyloxy” refers to a group, having the structure —OAr, in which Ar is a heteroatom-substituted Cn-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cn-acyloxy, heteroatom-substituted Cn-acyloxy, alkylcarbonyloxy, arylcarbonyloxy, alkoxycarbonyloxy, aryloxycarbonyloxy, and carboxylate groups. The term “heteroatom-unsubstituted Cn-acyloxy” refers to a group, having the structure —OAc, in which Ac is a heteroatom-unsubstituted Cn-acyl, as that term is defined above. For example, —OC(O)CH₃ is a non-limiting example of a heteroatom-unsubstituted acyloxy group. The term “heteroatom-substituted Cn-acyloxy” refers to a group, having the structure —OAc, in which Ac is a heteroatom-substituted Cn-acyl, as that term is defined above. For example, —OC(O)OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cn-alkylamino, and heteroatom-substituted Cn-alkylamino. The term “heteroatom-unsubstituted Cn-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C1-C10-alkylamino has 1 to 10 carbon atoms. The term “heteroatom-unsubstituted Cn-alkylamino” includes groups, having the structure —NHR, in which R is a heteroatom-unsubstituted Cn-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cn-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cn-alkylamino” includes groups, having the structure —NHR, in which R is a heteroatom-substituted Cn-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cn-alkenylamino, heteroatom-substituted Cn-alkenylamino, dialkenylamino, and alkyl(alkenyl)amino groups. The term “heteroatom-unsubstituted Cn-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C2-C10-alkenylamino has 2 to 10 carbon atoms. The term “heteroatom-unsubstituted Cn-alkenylamino” includes groups, having the structure —NHR, in which R is a heteroatom-unsubstituted Cn-alkenyl, as that term is defined above. The term “heteroatom-substituted Cn-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cn-alkenylamino” includes groups, having the structure —NHR, in which R is a heteroatom-substituted Cn-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cn-alkynylamino, heteroatom-substituted Cn-alkynylamino, dialkynylamino, alkyl(alkynyl)amino, and alkenyl(alkynyl)amino groups. The term “heteroatom-unsubstituted Cn-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C2-C10-alkynylamino has 2 to 10 carbon atoms. The term “heteroatom-unsubstituted Cn-alkynylamino” includes groups, having the structure —NHR, in which R is a heteroatom-unsubstituted Cn-alkynyl, as that term is defined above. The term “heteroatom-substituted Cn-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cn-alkynylamino” includes groups, having the structure —NHR, in which R is a heteroatom-substituted Cn-alkynyl, as that term is defined above.

The term “arylamino” includes heteroatom-unsubstituted arylamino, heteroatom-substituted arylamino, heteroatom-unsubstituted Cn-arylamino, heteroatom-substituted Cn-arylamino, heteroarylamino, heterocyclic arylamino, and alkyl(aryl)amino groups. The term “heteroatom-unsubstituted Cn-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cn-arylamino” includes groups, having the structure —NHR, in which R is a heteroatom-unsubstituted Cn-aryl, as that term is defined above. The term “heteroatom-substituted Cn-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cn-arylamino” includes groups, having the structure —NHR, in which R is a heteroatom-substituted Cn-aryl, as that term is defined above.

The term “aralkylamino” includes heteroatom-unsubstituted aralkylamino, heteroatom-substituted aralkylamino, heteroatom-unsubstituted Cn-aralkylamino, heteroatom-substituted Cn-aralkylamino, heteroaralkylamino, heterocyclic aralkylamino groups, and diaralkylamino groups. The term “heteroatom-unsubstituted Cn-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cn-aralkylamino” includes groups, having the structure —NHR, in which R is a heteroatom-unsubstituted Cn-aralkyl, as that term is defined above. The term “heteroatom-substituted Cn-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cn-aralkylamino” includes groups, having the structure —NHR, in which R is a heteroatom-substituted Cn-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cn-amido, heteroatom-substituted Cn-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cn-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cn-amido” includes groups, having the structure —NHR, in which R is a heteroatom-unsubstituted Cn-acyl, as that term is defined above. The group, —NHC(O)CH₃, is a non-limiting example of a heteroatom-unsubstituted amido group. The term “heteroatom-substituted Cn-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C1₀-amido has 1 to 10 carbon atoms. The term “heteroatom-substituted Cn-amido” includes groups, having the structure —NHR, in which R is a heteroatom-unsubstituted Cn-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cn-alkylthio, and heteroatom-substituted Cn-alkylthio. The term “heteroatom-unsubstituted Cn-alkylthio” refers to a group, having the structure —SR, in which R is a heteroatom-unsubstituted Cn-alkyl, as that term is defined above. The group, —SCH3, is an example of a heteroatom-unsubstituted alkylthio group. The term “heteroatom-substituted Cn-alkylthio” refers to a group, having the structure —SR, in which R is a heteroatom-substituted Cn-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cn-alkenylthio, and heteroatom-substituted Cn-alkenylthio. The term “heteroatom-unsubstituted Cn-alkenylthio” refers to a group, having the structure —SR, in which R is a heteroatom-unsubstituted Cn-alkenyl, as that term is defined above. The term “heteroatom-substituted Cn-alkenylthio” refers to a group, having the structure —SR, in which R is a heteroatom-substituted Cn-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cn-alkynylthio, and heteroatom-substituted Cn-alkynylthio. The term “heteroatom-unsubstituted Cn-alkynylthio” refers to a group, having the structure —SR, in which R is a heteroatom-unsubstituted Cn-alkynyl, as that term is defined above. The term “heteroatom-substituted Cn-alkynylthio” refers to a group, having the structure —SR, in which R is a heteroatom-substituted Cn-alkynyl, as that term is defined above.

The term “arylthio” includes heteroatom-unsubstituted arylthio, heteroatom-substituted arylthio, heteroatom-unsubstituted Cn-arylthio, heteroatom-substituted Cn-arylthio, heteroarylthio, and heterocyclic arylthio groups. The term “heteroatom-unsubstituted Cn-arylthio” refers to a group, having the structure —SAr, in which Ar is a heteroatom-unsubstituted Cn-aryl, as that term is defined above. The group, —SC₆H₅, is an example of a heteroatom-unsubstituted arylthio group. The term “heteroatom-substituted Cn-arylthio” refers to a group, having the structure —SAr, in which Ar is a heteroatom-substituted Cn-aryl, as that term is defined above.

The term “aralkylthio” includes heteroatom-unsubstituted aralkylthio, heteroatom-substituted aralkylthio, heteroatom-unsubstituted Cn-aralkylthio, heteroatom-substituted Cn-aralkylthio, heteroaralkylthio, and heterocyclic aralkylthio groups. The term “heteroatom-unsubstituted Cn-aralkylthio” refers to a group, having the structure —SAr, in which Ar is a heteroatom-unsubstituted Cn-aralkyl, as that term is defined above. The group, —SCH₂C₆H₅, is an example of a heteroatom-unsubstituted aralkyl group. The term “heteroatom-substituted Cn-aralkylthio” refers to a group, having the structure —SAr, in which Ar is a heteroatom-substituted Cn-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cn-acylthio, heteroatom-substituted Cn-acylthio, alkylcarbonyloxy, arylcarbonyloxy, alkoxycarbonyloxy, aryloxycarbonyloxy, and carboxylate groups. The term “heteroatom-unsubstituted Cn-acylthio” refers to a group, having the structure —SAc, in which Ac is a heteroatom-unsubstituted Cn-acyl, as that term is defined above. The group, —SCOCH₃, is an example of a heteroatom-unsubstituted acylthio group. The term “heteroatom-substituted Cn-acylthio” refers to a group, having the structure —SAc, in which Ac is a heteroatom-substituted Cn-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cn-alkylsilyl, and heteroatom-substituted Cn-alkylsilyl. The term “heteroatom-unsubstituted Cn-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C1-C10-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cn-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cn-phosphonate, and heteroatom-substituted Cn-phosphonate. The term “heteroatom-unsubstituted Cn-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C₀-C₁₀-phosphonate has 0 to 10 carbon atoms. The groups, —P(O)(OH)2, —P(O)(OH)OCH₃, —P(O)(OH)OCH₂CH₃, —P(O)(OCH₃)₂, and —P(O)(OH)(OC₆H₅) are non-limiting examples of heteroatom-unsubstituted phosphonate groups. The term “heteroatom-substituted Cn-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C₀-C₁₀-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cn-phosphinate, and heteroatom-substituted Cn-phosphinate. The term “heteroatom-unsubstituted Cn-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C0-C10-phosphinate has 0 to 10 carbon atoms. The groups, —P(O)(OH)H, —P(O)(OH)CH3, —P(O)(OH)CH2CH3, —P(O)(OCH3)CH3, and —P(O)(OC6H5)H are non-limiting examples of heteroatom-unsubstituted phosphinate groups. The term “heteroatom-substituted Cn-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C0-C10-phosphinate has 0 to 10 carbon atoms.

Any apparently unfulfilled valency is to be understood to be properly filled by hydrogen atom(s). For example, a compound with a substituent of —O or —N is to be understood to be —OH or —NH2, respectively.

Any genus, subgenus, or specific compound discussed herein is specifically contemplated as being excluded from any embodiment described herein.

Compounds described herein may be prepared synthetically using conventional organic chemistry methods known to those of skill in the art and/or are commercially available (e.g., ChemBridge Co., San Diego, CA).

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Derivatives of compounds of the present invention are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds employed in methods may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include 13C and 14C.

As noted above, compounds described herein may exist in prodrug form. As used herein, “prodrug” is intended to include any covalently bonded carriers which release the active parent drug or compounds that are metabolized in vivo to an active drug or other compounds employed in the methods described herein in vivo when such prodrug is administered to a subject. Since prodrugs are known to enhance numerous desirable qualities of pharmaceuticals (e.g., solubility, bioavailability, manufacturing, etc.), the compounds employed in some methods described herein may, if desired, be delivered in prodrug form. Thus, the invention contemplates prodrugs of compounds described herein as well as methods of delivering prodrugs. Prodrugs of the compounds employed in embodiments may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

It should be recognized that the particular anion or cation forming a part of any salt in any embodiment is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### C. Pharmaceutical Formulations and Administration Thereof

### 1. Pharmaceutical Formulations and Routes of Administration

Pharmaceutical compositions disclosed herein comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds administered according to embodiments disclosed herein may comprise different types of carriers depending on whether it is to be administered in solid, liquid or aerosol form, and whether it need to be sterile for such routes of administration as injection. Embodiments can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, systemically, subcutaneously, subconjunctival, intravesicularly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition of embodiments disclosed herein that are administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a compound of the present disclosure. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin, or combinations thereof.

### 2. Combination Therapy

In some embodiments, it is contemplated that the tolerance-inducing compositions disclosed herein may be used in conjunction with the compositions for which a tolerance is being induced as part of a treatment regimen. This process may involve contacting the cell(s) with the agents at the same time or within a period of time wherein separate administration of the agents produces a desired therapeutic benefit. This may be achieved by contacting the cell, tissue or organism with a single composition or pharmacological formulation that includes two or more agents, or by contacting the cell with two or more distinct compositions or formulations, wherein one composition includes one agent and the other includes another.

Compounds discussed herein may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more tolerance-inducing compositions may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to administering the compositions for which a tolerance is being induced. In some embodiments, more than one course of therapy may be employed. It is contemplated that multiple courses may be implemented.

### D. Organisms and Cell Source

Cells that may be used in some methods can be from a variety of sources. Embodiments include the use of mammalian cells, such as cells from monkeys, chimpanzees, rabbits, mice, rats, ferrets, dogs, pigs, humans, and cows. Alternatively, the cells may be from fruit flies, yeast, or E. coli, which are all model systems for evaluating homologous recombination.

Methods can involve cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord.

Moreover, methods can be employed in cells of the following type: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

Moreover, methods can be implemented with or in plants or parts of plants, including fruit, flowers, leaves, stems, seeds, cuttings. Plants can be agricultural, medicinal, or decorative.

### E. Antigens

The antigen employed as X in the compositions of Formula 1, or in any of the compositions or methods of the current disclosure, can be a protein or a peptide, e.g. the antigen may be a complete or partial therapeutic agent, a full-length transplant protein or peptide thereof, a full-length autoantigen or peptide thereof, a full-length allergen or peptide thereof, and/or a nucleic acid, or a mimetic of an aforementioned antigen. Combinations of multiple fragments may also be used, depending on the embodiment. For example, if a longer peptide identified as P has antigenic regions A, B, C, and D, compositions disclosed herein for induction of tolerance to P can comprise any combination of A, B, C, and D, and repeats of any of A, B, C, and D. A listing of any particular antigen in a category or association with any particular disease or reaction does not preclude that antigen from being considered part of another category or associated with another disease or reaction.

In several embodiments, the antigen comprises one or more therapeutic agents that are proteins, peptides, antibodies and antibody-like molecules (including antibody fragments and fusion proteins with antibodies and antibody fragments), and gene therapy vectors. These include human, non-human (such as mouse) and non-natural (e.g., engineered) proteins, antibodies, chimeric antibodies, humanized antibodies, viruses and virus-like particles, and non-antibody binding scaffolds, such as fibronectins, DARPins, knottins, and the like. In several embodiments, human allograft transplantation antigens against which transplant recipients develop an unwanted immune response are used. In several embodiments, the antigen comprises one or more self-antigens that cause an unwanted, autoimmune response. While self-antigens are of an endogenous origin in an autoimmune disease patient, according to several embodiments, the polypeptides employed in the disclosed compositions are, depending on the embodiment, synthesized exogenously (as opposed to being purified and concentrated from a source of origin).

In several embodiments, the antigen to which tolerance is desired comprises one or more foreign antigens, such as food, animal, plant and environmental antigens, against which a patient experiences an unwanted immune response. While a therapeutic protein can also be considered a foreign antigen due to its exogenous origin, for purposes of clarity in the description of the present disclosure such therapeutics are described as a separate group. Similarly, a plant or an animal antigen can be eaten and considered a food antigen, and an environmental antigen may originate from a plant. They are, however, considered foreign antigens. In the interest of simplicity no attempt will be made to describe distinguish and define all of such potentially overlapping groups, as those skilled in the art can appreciate the antigens that can be employed in the compositions of the disclosure, particularly in light of the detailed description and examples.

In several embodiments, X is selected from the group consisting of insulin, proinsulin, preproinsulin, gluten, gliadin, myelin basic protein, myelin oligodendrocyte glycoprotein and proteolipid protein, Factor VIII, Factor IX, asparaginase, uricase and fragments of any of the preceding. In several embodiments, the antigen X is not a full length protein. For example, in some embodiments, the antigen is not full length gliadin, insulin, or proinsulin. In several embodiments, the antigen is not full length myelin basic protein, not full length myelin oligodendrocyte protein, or not full length proteolipid protein. In several embodiments, the antigen X is not a fragment of a protein. As discussed in more detail below, there exist a variety of antigens to which tolerance may be desired. These may include, but are not limited to, exogenous antigens that result in an adverse immune response when a subject is exposed to the antigen. In several embodiments, the adverse immune response could be a result of ingestion of the antigen, e.g., orally or nasally, or via some other mucosal route. These routes could be the case, for example, with food antigens. In some embodiments, the antigen may be purposefully administered to a subject, for example, with the administration of a therapeutic composition to treat a disease or condition that the subject is affected by. In still additional embodiments, the antigen may be produced by the subject, e.g., an autoimmune antigen. For example, in several embodiments, X comprises a foreign transplant antigen against which transplant recipients develop an unwanted immune response or a tolerogenic portion thereof. In several embodiments, X comprises a foreign food, animal, plant or environmental antigen against which patients develop an unwanted immune response or a tolerogenic portion thereof. In several embodiments, X comprises a foreign therapeutic agent against which patients develop an unwanted immune response or a tolerogenic portion thereof. In several embodiments, X comprises a synthetic self-antigen against the endogenous version of which patients develop an unwanted immune response or a tolerogenic portion thereof.

In further detail to the above, there are provided in several embodiments, compounds where X is a food antigen. In some such embodiments, X is one or more of conarachin (Ara h 1), allergen II (Ara h 2), arachis agglutinin, conglutin (Ara h 6), a-lactalbumin (ALA), lactotransferrin, Pen a 1 allergen (Pen a 1), allergen Pen m 2 (Pen m 2), tropomyosin fast isoform, high molecular weight glutenin, low molecular weight glutenin, alpha-gliadin, gamma-gliadin, omega-gliadin, hordein, seclain, and avenin. Fragment of any of these antigens and/or mimotopes of any of these antigens are also used, in several embodiments. In several embodiments, X is selected from the group consisting of gluten, high molecular weight glutenin, low molecular weight glutenin, alpha-gliadin, gamma-gliadin, omega-gliadin, hordein, seclain, and avenin and fragments thereof. In several embodiments, X is selected from the group consisting of gluten, high molecular weight glutenin, low molecular weight glutenin, alpha-gliadin, gamma-gliadin, and omega-gliadin and fragments thereof. In several embodiments, X is gluten or fragment thereof. In several embodiments, X is gliadin or fragment thereof.

In several embodiments, there are provided compounds where X is a therapeutic agent. In several embodiments, X is selected from the group consisting of Factor VII, Factor IX, asparaginase, and uricase and fragments thereof. In several embodiments, X is a therapeutic agent selected from the group consisting of Factor VII and Factor IX and fragments thereof. In several embodiments, X is a therapeutic agent selected from the group consisting of Factor VIII or fragment thereof. In several embodiments, when X is a therapeutic agent, the compound can be used in the treatment, prevention, reduction, or otherwise amelioration of an immune response developed against a therapeutic agent for hemophilia. As discussed herein, mimotopes of any antigenic portion of the antigens above can be used in several embodiments.

In several embodiments, X comprises asparaginase or a fragment thereof. In several embodiments, X comprises uricase or a fragment thereof. In several such embodiments, the compound can be used in the treatment, prevention, reduction, or otherwise amelioration of an immune response developed against an anti-neoplastic agent. As discussed herein, mimotopes of any antigenic portion of the antigens above can be used in several embodiments.

In several embodiments, X is associated with an autoimmune disease. For example, in several embodiments, the associated autoimmune disease is one or more of Type I diabetes, multiple sclerosis, rheumatoid arthritis, vitiligo, uveitis, pemphis vulgaris and neuromyelitis optica.

In several embodiments, the autoimmune disease is Type I diabetes and X comprises insulin or a fragment thereof. In several embodiments, the autoimmune disease is Type I diabetes and X comprises proinsulin or a fragment thereof. In several embodiments, the autoimmune disease is Type I diabetes and X comprises preproinsulin or a fragment thereof. As discussed herein, mimotopes of any antigenic portion of the antigens above can be used in several embodiments. In several embodiments, combinations of these antigens can be incorporated into the tolerogenic compound which may aid in reducing immune responses to self-antigens at multiple points along the insulin pathway.

In several embodiments, the autoimmune disease is multiple sclerosis and X comprises myelin basic protein or a fragment thereof. In several embodiments, the autoimmune disease is multiple sclerosis and X comprises myelin oligodendrocyte glycoprotein or a fragment thereof. In several embodiments, the autoimmune disease is multiple sclerosis and X comprises proteolipid protein or a fragment thereof. As discussed herein, mimotopes of any antigenic portion of the antigens above can be used in several embodiments. In several embodiments, combinations of these antigens can be incorporated into the tolerogenic compound (e.g., a mixture of antigens or fragments of MOG, MBP and/or PLP) which may aid in reducing immune responses to self-antigens at multiple points along the enzymatic pathways that control myelination or myelin repair.

As discussed herein, mimotopes of any antigenic portion of the self-antigens above (or otherwise disclosed herein) can be used in several embodiments.

In several embodiments, the pharmaceutically acceptable composition consists of, or consists essentially of a compound wherein X is a food antigen, therapeutic agent, a self antigen, or fragment thereof, a linker Y, and a liver targeting moiety Z selected from mannose and/or a mannose receptor-targeting moiety (including, but not limited to, one or more of mannosamine, N-acetylmannosamine, or N-acetylglucosamine).

The tolerogenic antigen can be a complete protein, a portion of a complete protein, a peptide, or the like, and can be derivatized (as discussed above) for attachment to a linker and/or antigen-binding moiety, can be a variant and/or can contain conservative substitutions, particularly maintaining sequence identity, and/or can be desialylated.

In the embodiments where the antigen is a therapeutic protein, peptide, antibody or antibody-like molecule, specific antigens can be selected from: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor VIIa, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin [including recombinant human insulin (“rHu insulin”) and bovine insulin], Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Natalizumab, Octreotide, Ofatumumab, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, vW factor. The therapeutic protein can be obtained from natural sources (e.g., concentrated and purified) or synthesized, e.g., recombinantly, and includes antibody therapeutics that are typically IgG monoclonal or fragments or fusions.

Particular therapeutic protein, peptide, antibody or antibody-like molecules include, but are not limited to, Abciximab, Adalimumab, Agalsidase alfa, Agalsidase beta, Aldeslukin, Alglucosidase alfa, Factor VIII, Factor IX, Infliximab, Insulin (including rHu Insulin), L-asparaginase, Laronidase, Natalizumab, Octreotide, Phenylalanine ammonia-lyase (PAL), or Rasburicase (uricase) and generally IgG monoclonal antibodies in their varying formats.

Some embodiments employ hemostatic agents (e.g., Factor VIII and IX), Insulin (including rHu Insulin), and the non-human therapeutics uricase, PAL and asparaginase.

In several embodiments, therapeutic agents are delivered through the use of, e.g., a gene therapy vector. In some such embodiments, an immune response may be developed against a portion of such vectors and/or their cargo (e.g., the therapeutic agent). Thus, in several embodiments, the antigen to which tolerance is desired comprises a gene therapy vector, including, but are not limited to: adenoviruses and adeno-associated virus (and corresponding variants −1, −2, −5, −6, −8, −9, and/or other parvoviruses), lentiviruses, and retroviruses.

Unwanted immune response in hematology and transplant includes autoimmune aplastic anemia, transplant rejection (generally), and Graft vs. Host Disease (bone marrow transplant rejection). In the embodiments where the tolerogenic antigen is a human allograft transplantation antigen, specific sequences can be selected from: subunits of the various MHC class I and MHC class II haplotype proteins (for example, donor/recipient differences identified in tissue cross-matching), and single-amino-acid polymorphisms on minor blood group antigens including RhCE, Kell, Kidd, Duffy and Ss. Such compositions can be prepared individually for a given donor/recipient pair.

In type 1 diabetes mellitus, antigens include, but are not limited to: insulin, proinsulin, preproinsulin, glutamic acid decarboxylase-65 (GAD-65 or glutamate decarboxylase 2), GAD-67, glucose-6 phosphatase 2 (IGRP or islet-specific glucose 6 phosphatase catalytic subunit related protein), insulinoma-associated protein 2 (IA-2), and insulinoma-associated protein 2p (IA-2p); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100P, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5, or immunogenic fragments or portions of any of such antigens. It should be noted that insulin is an example of an antigen that can be characterized both as a self-antigen and a therapeutic protein antigen. For example, rHu Insulin and bovine insulin are therapeutic protein antigens (that are the subject of unwanted immune attack), whereas endogenous human insulin is a self-antigen (that is the subject of an unwanted immune attack). Because endogenous human insulin is not available to be employed in a pharmaceutical composition, a recombinant form is employed in certain embodiments of the compositions of the disclosure.

Human insulin, including an exogenously obtained form useful in the compositions of the disclosure, has the following sequence (UNIPROT P01308):

(SEQ ID NO: 1)
MALWMRLLPLLALLALWGPDPAAAFVNQHLCGSHLVEALYLVCGERGFF
YTPKTRREAEDLQVGQVELGGGPGAGSLQPLALEGSLQKRGIVEQCCTS
ICSLYQLENYCN.

GAD-65, including an exogenously obtained form useful in the compositions of the disclosure, has the following sequence (UNIPROT Q05329):

(SEQ ID NO: 2)
MASPGSGFWSFGSEDGSGDSENPGTARAWCQVAQKFTGGIGNKLCALLY
GDAEKPAESGGSQPPRAAARKAACACDQKPCSCSKVDVNYAFLHATDLL
PACDGERPTLAFLQDVMNILLQYVVKSFDRSTKVIDFHYPNELLQEYNW
ELADQPQNLEEILMHCQTTLKYAIKTGHPRYFNQLSTGLDMVGLAADWL
TSTANTNMFTYEIAPVFVLLEYVTLKKMREIIGWPGGSGDGIFSPGGAI
SNMYAMMIARFKMFPEVKEKGMAALPRLIAFTSEHSHFSLKKGAAALGI
GTDSVILIKCDERGKMIPSDLERRILEAKQKGFVPFLVSATAGTTVYGA
FDPLLAVADICKKYKIWMHVDAAWGGGLLMSRKHKWKLSGVERANSVTW
NPHKMMGVPLQCSALLVREEGLMQNCNQMHASYLFQQDKHYDLSYDTGD
KALQCGRHVDVFKLWLMWRAKGTTGFEAHVDKCLELAEYLYNIIKNREG
YEMVFDGKPQHTNVCFWYIPPSLRTLEDNEERMSRLSKVAPVIKARMME
YGTTMVSYQPLGDKVNFFRMVISNPAATHQDIDFLIEEIERLGQDL.

IGRP, including an exogenously obtained form useful in the compositions of the disclosure, has the following sequence (UNIPROT QN9QR9):

(SEQ ID NO: 3)
MDFLHRNGVLIIQHLQKDYRAYYTFLNFMSNVGDPRNIFFIYFPLCFQF
NQTVGTKMIWVAVIGDWLNLIFKWILFGHRPYWWVQETQIYPNHSSPCL
EQFPTTCETGPGSPSGHAMGASCVWYVMVTAALSHTVCGMDKFSITLHR
LTWSFLWSVFWLIQISVCISRVFIATHEPHQVILGVIGGMLVAEAFEHT
PGIQTASLGTYLKTNLFLFLFAVGFYLLLRVLNIDLLWSVPIAKKWCAN
PDWIHIDTTPFAGLVRNLGVLFGLGFAINSEMFLLSCRGGNNYTLSFRL
LCALTSLTILQLYHFLQIPTHEEHLFYVLSFCKSASIPLTVVAFIPYSV
HMLMKQSGKKSQ.

In several embodiments, human proinsulin, including an exogenously obtained form useful in the tolerogenic compositions of the disclosure, has the following sequence:

(SEQ ID NO: 4)
FVNQHLCGSHLVEALYLVCGERGFFYTPKTRREAEDLQVGQVELGGGPG
AGSLQPLALEGSLQKRGIVEQCCTSICSLYQLENYCN.

Depending on the embodiment, peptides/epitopes useful in the tolerogenic compositions of the disclosure for treating type 1 diabetes include some or all of the following sequences, individually in a tolerogenic composition or together in a cocktail of tolerogenic compositions:

Human Proinsulin 1-70:
(SEQ ID NO: 5)
FVNQHLCGSHLVEALYLVCGERGFFYTPKTRREAEDLQVGQVELGGGPGA
GSLQPLALEGSLQKRGIVEQ;
Human Proinsulin 9-70:
(SEQ ID NO: 6)
SHLVEALYLVCGERGFFYTPKTRREAEDLQVGQVELGGGPGAGSLQPLAL
EGSLQKRGIVEQ;
Human Proinsulin 9-38:
(SEQ ID NO: 7)
SHLVEALYLVCGERGFFYTPKTRREAEDLQ;
Human Proinsulin 1-38:
(SEQ ID NO: 8)
FVNQHLCGSHLVEALYLVCGERGFFYTPKTRREAEDLQ;
Human Proinsulin 9-23:
(SEQ ID NO: 9)
SHLVEALYLVCGERG;
Human Proinsulin 45-71 (C13-A6):
(SEQ ID NO: 10)
GGGPGAGSLQPLALEGSLQKRGIVEQC;
Human Proinsulin C24-A1:
(SEQ ID NO: 11)
LALEGSLQKRG;
Human Proinsulin C19-A3:
(SEQ ID NO: 12)
GSLQPLALEGSLQKRGIV;
Human Proinsulin C13-32:
(SEQ ID NO: 13)
GGGPGAGSLQPLALEGSLQK;
Human Proinsulin B9-C4:
(SEQ ID NO: 14)
SHLVEALYLVCGERGFFYTPKTRREAED;
Human Proinsulin C22-A5:
(SEQ ID NO: 15)
QPLALEGSLQKRGIVEQ;
Human IA-2 718-782:
(SEQ ID NO: 16)
AYQAEPNTCATAQGEGNIKKNRHPDFLPYDHARIKLKVESSPSRSDYINA
SPIIEHDPRMPAYIA;
Human IA-2 785-819:
(SEQ ID NO: 17)
GPLSHTIADFWQMVWESGCTVIVMLTPLVEDGVKQ;
Human IA-2 828-883:
(SEQ ID NO: 18)
GASLYHVYEVNLVSEHIWCEDFLVRSFYLKNVQTQETRTLTQFHFLSWPA
EGTPAS;
Human IA-2 943-979:
(SEQ ID NO: 19)
EHVRDQRPGLVRSKDQFEFALTAVAEEVNAILKALPQCG.

In autoimmune diseases of the thyroid, including Hashimoto's thyroiditis and Graves' disease, main antigens include, but are not limited to, thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor.

In Addison's Disease, main antigens include, but are not limited to, 21-hydroxylase, 17α-hydroxylase, and P450 side chain cleavage enzyme (P450scc); other antigens include ACTH receptor, P450c21 and P450c17.

In premature ovarian failure, main antigens include, but are not limited to, FSH receptor and a-enolase.

In autoimmune hypophysitis, or pituitary autoimmune disease, main antigens include, but are not limited to, pituitary gland-specific protein factor (PGSF) 1a and 2; another antigen is type 2 iodothyronine deiodinase.

In multiple sclerosis, main antigens include, but are not limited to, myelin basic protein (“MBP”), myelin oligodendrocyte glycoprotein (“MOG”) and myelin proteolipid protein (“PLP”).

MBP, including an exogenously obtained form useful in the compositions of the disclosure, has the following sequence (UNIPROT P02686):

(SEQ ID NO: 20)
MGNHAGKRELNAEKASTNSETNRGESEKKRNLGELSRTTSEDNEVFGEAD
ANQNNGTSSQDTAVTDSKRTADPKNAWQDAHPADPGSRPHLIRLFSRDAP
GREDNTFKDRPSESDELQTIQEDSAATSESLDVMASQKRPSQRHGSKYLA
TASTMDHARHGFLPRHRDTGILDSIGRFFGGDRGAPKRGSGKDSHHPART
AHYGSLPQKSHGRTQDENPVVHFFKNIVTPRTPPPSQGKGRGLSLSRFSW
GAEGQRPGFGYGGRASDYKSAHKGFKGVDAQGTLSKIFKLGGRDSRSGSP
MARR

MOG, including an exogenously obtained form useful in the compositions of the disclosure, has the following sequence (UNIPROT Q16653):

(SEQ ID NO: 21)
MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLIFLCLQYRLRGKLRAEIENLHRTFDPH
FLRVPCWKITLFVIVPVLGPLVALIICYNWLHRRLAGQFLEELRNPF.

PLP, including an exogenously obtained form useful in the compositions of the disclosure, has the following sequence (UNIPROT P60201):

(SEQ ID NO: 22)
MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCHCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF.

Peptides/epitopes useful in the compositions of the disclosure for treating multiple sclerosis include some or all of the following sequences, individually in a tolerogenic composition as disclosed herein or together in a combination (e.g., a cocktail) of tolerogenic compositions:

MBP 13-32:
(SEQ ID NO: 23)
KYLATASTMDHARHGFLPRH;
MBP 83-99:
(SEQ ID NO: 24)
ENPWHFFKNIVTPRTP;
MBP 111-129:
(SEQ ID NO: 25)
LSRFSWGAEGQRPGFGYGG;
MBP 146-170:
(SEQ ID NO: 26)
AQGTLSKIFKLGGRDSRSGSPMARR;
MOG 1-20:
(SEQ ID NO: 27)
GQFRVIGPRHPIRALVGDEV;
MOG 35-55:
(SEQ ID NO: 28)
MEVGWYRPPFSRWHLYRNGK;
PLP 139-154:
(SEQ ID NO: 29)
HCLGKWLGHPDKFVGI;
MOG 30-60:
(SEQ ID NO: 30)
KNATGMEVGWYRSPFSRVVHLYRNGKDQDAE;
MBP 83-99:
(SEQ ID NO: 31)
ENPVVHFFKNIVTPRTP;
MOG 35-55:
(SEQ ID NO: 32)
MEVGWYRPPFSRVVHLYRNGK;
MBP 82-98:
(SEQ ID NO: 33)
DENPVVHFFKNIVTPRT;
MBP 82-99:
(SEQ ID NO: 34)
DENPVVHFFKNIVTPRTP;
MBP 82-106:
(SEQ ID NO: 35)
DENPVVHFFKNIVTPRTPPPSQGKG;
MBP 87-106:
(SEQ ID NO: 36)
VHFFKNIVTPRTPPPSQGKG;
MBP 131-155:
(SEQ ID NO: 37)
ASDYKSAHKGLKGVDAQGTLSKIFK;
PLP 41-58:
(SEQ ID NO: 38)
GTEKLIETYFSKNYQDYE;
PLP 89-106:
(SEQ ID NO: 39)
GFYTTGAVRQIFGDYKTT;
PLP 95-116:
(SEQ ID NO: 40)
AVRQIFGDYKTTICGKGLSATV;
PLP 178-197:
(SEQ ID NO: 41)
NTWTTCQSIAFPSKTSASIG;
PLP 190-209:
(SEQ ID NO: 42)
SKTSASIGSLCADARMYGVL;
MOG 11-30:
(SEQ ID NO: 43)
PIRALVGDEVELPCRISPGK;
MOG 21-40:
(SEQ ID NO: 44)
ELPCRISPGKNATGMEVGWY;
MOG 64-86:
(SEQ ID NO: 45)
EYRGRTELLKDAIGEGKVTLRIR;
MOG 1-62:
(SEQ ID NO. 46)
GQFRVIGPRHPIRALVGDEVELPCRISPGKNATGMEVGWYRPPFSRVVHL
YRNGKDQDGDQA
MBP 76-136:
(SEQ ID NO: 47)
SHGRTQDENPVVHFFKNIVTPRTPPPSQGKGRGLSLSRFSWGAEGQRPGF
GYGGRASDYKSCG

In rheumatoid arthritis, main antigens include, but are not limited to, collagen II, immunoglobulin binding protein, the fragment crystallizable region of immunoglobulin G, double-stranded DNA, and the natural and citrullinated forms of proteins implicated in rheumatoid arthritis pathology, including fibrin/fibrinogen, vimentin, collagen I and II, and alpha-enolase.

In autoimmune gastritis, a main antigen is H+,K+-ATPase.

In pernicious angemis, a main antigen is intrinsic factor.

In celiac disease, main antigens include, but are not limited to, tissue transglutaminase and the natural and deamidated forms of gluten or gluten-like proteins, such as alpha-, gamma-, and omega-gliadin, glutenin, hordein, secalin, and avenin. Those skilled in the art will appreciate, for example, that while the main antigen of celiac disease is alpha gliadin, alpha gliadin turns more immunogenic in the body through deamidation by tissue glutaminase converting alpha gliadin's glutamines to glutamic acid. Thus, while alpha gliadin is originally a foreign food antigen, once it has been modified in the body to become more immunogenic it can be characterized as a self-antigen, depending on the embodiment.

In vitiligo, a main antigen is tyrosinase, and tyrosinase related protein 1 and 2.

MART1, Melanoma antigen recognized by T cells 1, Melan-A, including an exogenously obtained form useful in the compositions of the disclosure, has the following sequence (UNIPROT Q16655):

(SEQ ID NO: 48)
MPREDAHFIYGYPKKGHGHSYTTAEEAAGIGILTVILGVLLLIGCWYCRR
RNGYRALMDKSLHVGTQCALTRRCPQEGFDHRDSKVSLQEKNCEPVVPNA
PPAYEKLSAEQSPPPYSP.

Tyrosinase, including an exogenously obtained form useful in the compositions of the disclosure, has the following sequence (UNIPROT P14679):

(SEQ ID NO: 49)
MLLAVLYCLLWSFQTSAGHFPRACVSSKNLMEKECCPPWSGDRSPCGQLS
GRGSCQNILLSNAPLGPQFPFTGVDDRESWPSVFYNRTCQCSGNFMGFNC
GNCKFGFWGPNCTERRLLVRRNIFDLSAPEKDKFFAYLTLAKHTISSDYV
IPIGTYGQMKNGSTPMENDINIYDLFVWMHYYVSMDALLGGSEIWRDIDF
AHEAPAFLPWHRLFLLRWEQEIQKLTGDENFTIPYWDWRDAEKCDICTDE
YMGGQHPTNPNLLSPASFFSSWQIVCSRLEEYNSHQSLCNGTPEGPLRRN
PGNHDKSRTPRLPSSADVEFCLSLTQYESGSMDKAANFSFRNTLEGFASP
LTGIADASQSSMHNALHIYMNGTMSQVQGSANDPIFLLHHAFVDSIFEQW
LRRHRPLQEVYPEANAPIGHNRESYMVPFIPLYRNGDFFISSKDLGYDYS
YLQDSDPDSFQDYIKSYLEQASRIWSWLLGAAMVGAVLTALLAGLVSLLC
RHKRKQLPEEKQPLLMEKEDYHSLYQSHL.

Melanocyte protein PMEL, gp100, including an exogenously obtained form useful in the compositions of the disclosure, has the following sequence (UNIPROT P40967):

(SEQ ID NO: 50)
MDLVLKRCLLHLAVIGALLAVGATKVPRNQDWLGVSRQLRTKAWNRQLYP
EWTEAQRLDCWRGGQVSLKVSNDGPTLIGANASFSIALNFPGSQKVLPDG
QVIWVNNTIINGSQVWGGQPVYPQETDDACIFPDGGPCPSGSWSQKRSFV
YVWKTWGQYWQVLGGPVSGLSIGTGRAMLGTHTMEVTVYHRRGSRSYVPL
AHSSSAFTITDQVPFSVSVSQLRALDGGNKHFLRNQPLTFALQLHDPSGY
LAEADLSYTWDFGDSSGTLISRALVVTHTYLEPGPVTAQVVLQAAIPLTS
CGSSPVPGTTDGHRPTAEAPNTTAGQVPTTEVVGTTPGQAPTAEPSGTTS
VQVPTTEVISTAPVQMPTAESTGMTPEKVPVSEVMGTTLAEMSTPEATGM
TPAEVSIVVLSGTTAAQVTTTEWVETTARELPIPEPEGPDASSIMSTESI
TGSLGPLLDGTATLRLVKRQVPLDCVLYRYGSFSVTLDIVQGIESAEILQ
AVPSGEGDAFELTVSCQGGLPKEACMEISSPGCQPPAQRLCQPVLPSPAC
QLVLHQILKGGSGTYCLNVSLADTNSLAVVSTQLIMPGQEAGLGQVPLIV
GILLVLMAVVLASLIYRRRLMKQDFSVPQLPHSSSHWLRLPRIFCSCPIG
ENSPLLSGQQV.

In myasthenia gravis, a main antigen is acetylcholine receptor.

In pemphigus vulgaris and variants, main antigens include, but are not limited to, desmoglein 3, 1 and 4; other antigens include pemphaxin, desmocollins, plakoglobin, perplakin, desmoplakins, and acetylcholine receptor.

In bullous pemphigoid, main antigens include BP180 and BP230; other antigens include plectin and laminin 5.

In dermatitis herpetiformis Duhring, main antigens include, but are not limited to, endomysium and tissue transglutaminase.

In epidermolysis bullosa acquisita, a main antigen is collagen VII.

In systemic sclerosis, main antigens include, but are not limited to, matrix metalloproteinase 1 and 3, the collagen-specific molecular chaperone heat-shock protein 47, fibrillin-1, and PDGF receptor; other antigens include Scl-70, U1 RNP, Th/To, Ku, Jo1, NAG-2, centromere proteins, topoisomerase I, nucleolar proteins, RNA polymerase I, II and III, PM-Slc, fibrillarin, and B23.

In mixed connective tissue disease, a main antigen is U1snRNP.

In Sjogren's syndrome, the main antigens include, but are not limited to, nuclear antigens SS-A and SS—B; other antigens include fodrin, poly(ADP-ribose) polymerase and topoisomerase, muscarinic receptors, and the Fc-gamma receptor IIIb.

In systemic lupus erythematosus, main antigens include nuclear proteins including the “Smith antigen,” SS-A, high mobility group box 1 (HMGB1), nucleosomes, histone proteins and double-stranded DNA (against which auto-antibodies are made in the disease process).

In Goodpasture's syndrome, main antigens include, but are not limited to, glomerular basement membrane proteins including collagen IV.

In rheumatic heart disease, a main antigen is cardiac myosin.

In autoimmune polyendocrine syndrome type 1 antigens include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In neuromyelitis optica, a main antigen is AQP4.

Aquaporin-4, including an exogenously obtained form useful in the compositions of the disclosure, has the following sequence (UNIPROT P55087):

(SEQ ID NO: 51)
MSDRPTARRWGKCGPLCTRENIMVAFKGVWTQAFWKAVTAEFLAMLIFVL
LSLGSTINWGGTEKPLPVDMVLISLCFGLSIATMVQCFGHISGGHINPAV
TVAMVCTRKISIAKSVFYIAAQCLGAIIGAGILYLVTPPSVVGGLGVTMV
HGNLTAGHGLLVELIITFQLVFTIFASCDSKRTDVTGSIALAIGFSVAIG
HLFAINYTGASMNPARSFGPAVIMGNWENHWIYWVGPIIGAVLAGGLYEY
VFCPDVEFKRRFKEAFSKAAQQTKGSYMEVEDNRSQVETDDLILKPGVVH
VIDVDRGEEKKGKDQSGEVLSSV.

In uveitis, main antigens include Retinal S-antigen or “S-arrestin” and interphotoreceptor retinoid binding protein (IRBP) or retinol-binding protein 3.

S-arrestin, including an exogenously obtained form useful in the compositions of the disclosure, has the following sequence (UNIPROT P10523):

(SEQ ID NO: 52)
MAASGKTSKSEPNHVIFKKISRDKSVTIYLGNRDYIDHVSQVQPVDGVVL
VDPDLVKGKKVYVTLTCAFRYGQEDIDVIGLTFRRDLYFSRVQVYPPVGA
ASTPTKLQESLLKKLGSNTYPFLLTFPDYLPCSVMLQPAPQDSGKSCGVD
FEVKAFATDSTDAEEDKIPKKSSVRLLIRKVQHAPLEMGPQPRAEAAWQF
FMSDKPLHLAVSLNKEIYFHGEPIPVTVTVTNNTEKTVKKIKAFVEQVAN
VVLYSSDYYVKPVAMEEAQEKVPPNSTLTKTLTLLPLLANNRERRGIALD
GKIKHEDTNLASSTIIKEGIDRTVLGILVSYQIKVKLTVSGFLGELTSSE
VATEVPFRLMHPQPEDPAKESYQDANLVFEEFARHNLKDAGEAEEGKRDK
NDVDE.

IRBP, including an exogenously obtained form useful in the compositions of the disclosure, has the following sequence (UNIPROT P10745):

(SEQ ID NO: 53)
MMREWVLLMSVLLCGLAGPTHLFQPSLVLDMAKVLLDNYCFPENLLGMQE
AIQQAIKSHEILSISDPQTLASVLTAGVQSSLNDPRLVISYEPSTPEPPP
QVPALTSLSEEELLAWLQRGLRHEVLEGNVGYLRVDSVPGQEVLSMMGEF
LVAHVWGNLMGTSALVLDLRHCTGGQVSGIPYIISYLHPGNTILHVDTIY
NRPSNTTTEIWTLPQVLGERYGADKDVVVLTSSQTRGVAEDIAHILKQMR
RAIVVGERTGGGALDLRKLRIGESDFFFTVPVSRSLGPLGGGSQTWEGSG
VLPCVGTPAEQALEKALAILTLRSALPGVVHCLQEVLKDYYTLVDRVPTL
LQHLASMDFSTVVSEEDLVTKLNAGLQAASEDPRLLVRAIGPTETPSWPA
PDAAAEDSPGVAPELPEDEAIRQALVDSVFQVSVLPGNVGYLRFDSFADA
SVLGVLAPYVLRQVWEPLQDTEHLIMDLRHNPGGPSSAVPLLLSYFQGPE
AGPVHLFTTYDRRTNITQEHFSHMELPGPRYSTQRGVYLLTSHRTATAAE
EFAFLMQSLGWATLVGEITAGNLLHTRTVPLLDTPEGSLALTVPVLTFID
NHGEAWLGGGVVPDAIVLAEEALDKAQEVLEFHQSLGALVEGTGHLLEAH
YARPEVVGQTSALLRAKLAQGAYRTAVDLESLASQLTADLQEVSGDHRLL
VFHSPGELVVEEAPPPPPAVPSPEELTYLIEALFKTEVLPGQLGYLRFDA
MAELETVKAVGPQLVRLVWQQLVDTAALVIDLRYNPGSYSTAIPLLCSYF
FEAEPRQHLYSVFDRATSKVTEVWTLPQVAGQRYGSHKDLYILMSHTSGS
AAEAFAHTMQDLQRATVIGEPTAGGALSVGIYQVGSSPLYASMPTQMAMS
ATTGKAWDLAGVEPDITVPMSEALSIAQDIVALRAKVPTVLQTAGKLVAD
NYASAELGAKMATKLSGLQSRYSRVTSEVALAEILGADLQMLSGDPHLKA
AHIPENAKDRIPGIVPMQIPSPEVFEELIKFSFHTNVLEDNIGYLRFDMF
GDGELLTQVSRLLVEHIWKKIMHTDAMIIDMRFNIGGPTSSIPILCSYFF
DEGPPVLLDKIYSRPDDSVSELWTHAQVVGERYGSKKSMVILTSSVTAGT
AEEFTYIMKRLGRALVIGEVTSGGCQPPQTYHVDDTNLYLTIPTARSVGA
SDGSSWEGVGVTPHVVVPAEEALARAKEMLQHNQLRVKRSPGLQDHL.

In the embodiments where the tolerogenic antigen is a foreign antigen against which an unwanted immune response can be developed, such as food antigens, specific antigens include, but are not limited to: from peanut: conarachin (Ara h 1), allergen II (Ara h 2), arachis agglutinin, conglutin (Ara h 6); conarachin, for example has the sequence identified as UNIPROT Q6PSU6; from apple: 31 kda major allergen/disease resistance protein homolog (Mal d 2), lipid transfer protein precursor (Mal d 3), major allergen Mal d 1.03D (Mal d 1); from milk: a-lactalbumin (ALA), lactotransferrin; from kiwi: actinidin (Act c 1, Act d 1), phytocystatin, thaumatin-like protein (Act d 2), kiwellin (Act d 5); from egg whites: ovomucoid, ovalbumin, ovotransferrin, and lysozyme; from egg yolks: livetin, apovitillin, and vosvetin; from mustard: 2S albumin (Sin a 1), 11S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cereals: high molecular weight glutenin, low molecular weight glutenin, alpha-, gamma- and omega-gliadin, hordein, secalin and/or avenin; peptides/epitopes useful in the compositions of the disclosure for treating Celiac Disease include some or all of the following sequences, individually in a composition of Formula 1 or together in a cocktail of compositions of Formula 1:

DQ-2 relevant, Alpha-gliadin “33-mer” native:
(SEQ ID NO: 54)
LQLQPFPQPQLPYPQPQLPYPQPQLPYPQPQPF;
DQ-2 relevant, Alpha-gliadin “33-mer” deamidated:
(SEQ ID NO: 55)
LQLQPFPQPELPYPQPELPYPQPELPYPQPQPF;
DQ-8 relevant, Alpha-gliadin:
(SEQ ID NO: 56)
QQYPSGQGSFQPSQQNPQ;
DQ-8 relevant, Omega-gliadin (wheat, U5UA46):
(SEQ ID NO: 57)
QPFPQPEQPFPW;
Alpha-gliadin “15-mer” fragment:
(SEQ ID NO: 58)
ELQPFPQPELPYPQP;
Gliadin linker:
(SEQ ID NO: 59)
GCRGGGPQPQPFPSQQPY;
Gliadin extended:
(SEQ ID NO: 60)
GCRGGGPQPQPFPSQQPYLQLQPFPQPQLPYPQPQLPYPQPQLPYPQPQ
PF;
Gliadin deamidated extended:
(SEQ ID NO: 61)
GCRGGGPQPQPFPSQQPYLQLQPFPQPELPYPQPELPYPQPELPYPQPQ
PF;
from strawberry: major strawberry allergy Fra a
1-E (Fra a 1); and from banana: profilin
(Mus xp 1).

In the embodiments where the antigen is a foreign antigen against which an unwanted immune response is developed, such as to animal, plant and environmental antigens, specific antigens can, for example, be: cat, mouse, dog, horse, bee, dust, tree and goldenrod, including the following proteins or peptides derived from: weeds, (including ragweed allergens amb a 1, 2, 3, 5, and 6, and Amb t 5; pigweed Che a 2 and 5; and other weed allergens Par j 1, 2, and 3, and Par o 1); grass (including major allergens Cyn d 1, 7, and 12; Dac g 1, 2, and 5; Hol I 1.01203; Lol p 1, 2, 3, 5, and 11; Mer a 1; Pha a 1; Poa p 1 and 5); pollen from ragweed and other weeds (including curly dock, lambs quarters, pigweed, plantain, sheep sorrel, and sagebrush), grass (including Bermuda, Johnson, Kentucky, Orchard, Sweet vernal, and Timothy grass), and trees (including catalpa, elm, hickory, olive, pecan, sycamore, and walnut); dust (including major allergens from species Dermatophagoides pteronyssinus, such as Der p 1, 2, 3, 4, 5, 6, 7, 8, 9, 10, 11, 14, 15, 18, 20, 21, and 23; from species Dermatophagoides farina, such as Der f 1, 2, 3, 6, 7, 10, 11, 13, 14, 15, 16, 18, 22, and 24; from species Blomia tropicalis such as Blo t 1, 2, 3, 4, 5, 6, 10, 11, 12, 13, 19, and 21; also allergens Eur m 2 from Euroglyphus maynei, Tyr p 13 from Tyrophagus putrescentiae, and allergens Bla g 1, 2, and 4; Per a 1, 3, and 7 from cockroach); pets (including cats, dogs, rodents, and farm animals; major cat allergens include Fel d 1 through 8, cat IgA, BLa g 2, and cat albumin; major dog allergens include Can f 1 through 6, and dog albumin); bee stings, including major allergens Api m 1 through 12; and fungus, including allergens derived from, species of Aspergillus and Penicillium, as well as the species Alternaria alternata, Davidiella tassiana, and Trichophyton rubrum.

In Parkinson's disease, the main antigen is alpha synuclein. Alpha synuclein, including an exogenously obtained form useful in the tolerogenic compositions of the disclosure, has the following sequence (UNIPROT P37840):

(SEQ ID NO: 62)
MDVFMKGLSKAKEGVVAAAEKTKQGVAEAAGKTKEGVLYVGSKTKEGVVH
GVATVAEKTKEQVTNVGGAVVTGVTAVAQKTVEGAGSIAAATGFVKKDQL
GKNEEGAPQEGILEDMPVDPDNEAYEMPSEEGYQDYEPEA.

The antigen can be a complete protein, a portion of a complete protein, a peptide, or the like, and can be derivatized (as discussed above) for attachment to a linker and/or mannosylating moiety, can be a variant and/or can contain conservative substitutions, particularly maintaining sequence identity, and/or can be desialylated.

### F. Examples

The following examples are included to demonstrate non-limiting embodiments of the invention disclosed herein. It should be appreciated by those of skill in the art that the techniques disclosed in the examples which follow represent techniques discovered by the inventor to function well in the practice of the embodiments of the invention. However, those of skill in the art should, in light of the present disclosure, appreciate that many changes can be made in the specific embodiments which are disclosed and still obtain a like or similar result without departing from the spirit and scope of the embodiments of the invention disclosed herein.

### Example 1

### Polymer and Conjugate Synthesis

All reactions were carried out at room temperature unless specified. Unless otherwise stated, chemicals were reagent grade and purchased from Sigma-Aldrich (Saint Louis, MO, USA). Size exclusion chromatography was carried out on an ÄKTA protein purification system (General Electric Healthcare Lifesciences), using a Superdex 200 10/300 column (General Electric Healthcare Lifesciences). All NMR spectra were collected on a Bruker Avance-II 400 MHz NMR, unless otherwise noted, and NMR spectra were analyzed with MnovaNMR (Mestrelab). High pressure size exclusion chromatography was performed on a Dionex Ultimate 3000 UHPLC (Thermo Fisher Scientific). Gels were imaged using a Biorad Universal Hood Gel Doc 2000 System (Biorad). Antigens conjugated to polymers as disclosed in the Examples are non-limiting examples of tolerogenic antigens according to embodiments disclosed herein, including embodiments using immunogenic fragments of antigens.

### Compound 1: 1-(2-chloroethyl)-α-D-mannose

Acetyl chloride (4.35 mL, 61.05 mmol) was added dropwise to the ice-cold solution of D-mannose (10.0 g, 55.51 mmol) in chloroethanol (40 mL, 413.68 mmol). The mixture was stirred for 15 minutes at 4° C. and then was transferred to the oil bath at 70° C. The reaction was then stirred for 4 h. After cooling to room temperature, a dark brown solution was poured into a 400 mL solution of ethyl acetate and DCM (3:1, v/v) in order to remove excess chloroethanol. The mixture was placed at −20° C. for 30 minutes and then a dark brown sticky precipitate was collected from the supernatant. The precipitate was dissolved in anhydrous ethanol and 3 spoons of activated charcoal was added. The suspension was mixed for 1.5 h and then filtered through Celite and washed with ethanol. In the last step, ethanol was evaporated via rotary evaporation to provide 12.8 g (95.24%) of product: C₈H₁₅ClO₆, ESI-MS [M+Na]⁺ₜₕₑₒᵣ=m/z 265.0455, [M+Na]⁺_{found}=m/z 265.0458; ¹H NMR (400 MHz, D₂O) δ 4.86 (s, C1, 1H), 3.92 (dd, C2, 1H), 3.62-3.85 (multiple signals from 7H: C3, C4, C5, C6, C6′, ethoxy group); ¹³C NMR (100 MHz, D₂O) δ 99.84, 76.32, 72.94, 70.52, 69.94, 69.65, 67.76, 66.75, 60.96, 43.39.

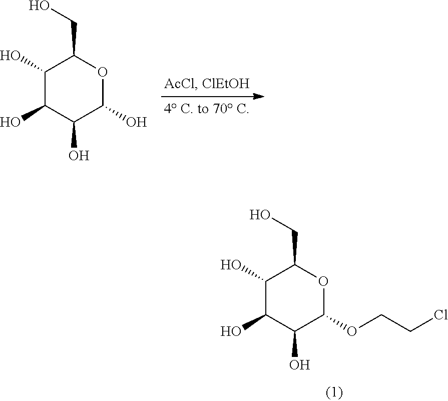 [SMILES: OC[C@H]1O[C@H](O)[C@@H](O)[C@@H](O)[C@@H]1O ; OC[C@H]1O[C@H](OCCCl)[C@@H](O)[C@@H](O)[C@@H]1O]

### Compound 2: 1-(2-azidoethyl)-α-D-mannose

Compound 1 (12.7 g, 52.48 mmol) was dissolved in 15 mL of N,N-dimethylformamide. To that solution, sodium azide was added (5.0 g, 76.92 mmol) and the suspension was placed in an oil bath and stirred over night at 90° C. After 16 h, the reaction mixture was filtered through Celite and the solvent was then removed via rotary evaporation to provide a oily, brown substance. The residual was adsorbed on silica gel and purified using flash chromatography (DCM:MeOH 92:8, v/v) to yield 5.6 g (42.86%) of pure product: C₈H₁₅N₃O₆, ESI-MS [M+Na]⁺ₜₕₑₒᵣ=m/z 272.2578, [M+Na]⁺_{found}=m/z 272.0850; ¹H NMR (400 MHz, D₂O) δ 4.84 (s, C1, 1H), 3.91 (dd, C2, 1H), 3.39-3.87 (multiple signals from 7H: C3, C4, C5, C6, C6′, ethoxy group); ¹³C NMR (100 MHz, D₂O) δ 99.85, 72.94, 70.44, 69.98, 66.73, 66.34, 50.24.

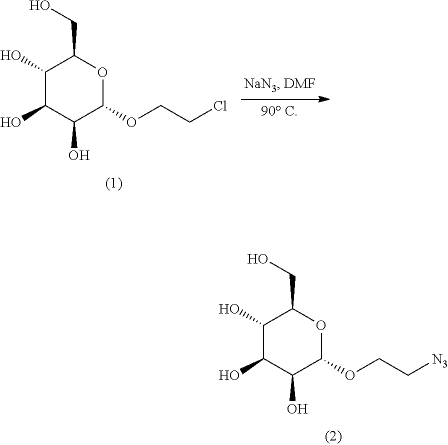 [SMILES: OC[C@H]1O[C@H](OCCCl)[C@@H](O)[C@@H](O)[C@@H]1O ; [N-]=[N+]=NCCO[C@H]1O[C@H](CO)[C@@H](O)[C@H](O)[C@@H]1O]

### Compound 3: 1-(2-aminoethyl)-α-D-mannose

A suspension of 2 (5.5 g, 22.09 mmol) and 10% palladium on carbon (one spoon, ca. 500 mg) in 10 mL of ethanol was hydrogenated in a Shlenk flask with an initial pressure of 2 bars of hydrogen gas. The reduction process was monitored by TLC. After 3 h reaction was completed and the suspension was filtered through Celite. The solvent was evaporated in vacuo to give 4.9 g (99.48%) of product: C8H17NO6, ESI-MS [M+Na]+theor=m/z 246.0954, [M+Na]+found=m/z 246.0955. 1H NMR (400 MHz, D2O) δ 4.80 (s, C1, 1H), 3.89 (dd, C2, 1H), 3.83-3.44 (multiple signals from 7H: C3, C4, C5, C6, C6′, ethoxy group); 13C NMR (100 MHz, D2O) δ 99.87, 72.74, 70.55, 70.03, 68.82, 66.81, 60.97, 39.94.

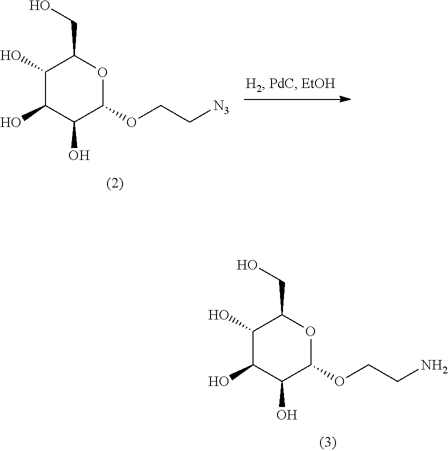 [SMILES: [N-]=[N+]=NCCO[C@H]1O[C@H](CO)[C@@H](O)[C@H](O)[C@@H]1O ; NCCO[C@H]1O[C@H](CO)[C@@H](O)[C@H](O)[C@@H]1O]

### Compound 4: N-[2-(α-D-mannose)ethyl]methacrylamide

Compound 3 (4.5 g, 20.17 mmol) was dissolved in 10 mL of N,N-dimethylformamide. To that solution, triethylamine (3 mL, 22.28 mmol) was added and the mixture was cooled down to 4° C. Subsequently, pentafluorophenyl methacrylate (4.38 mL, 24.21 mmol) was added drop-wise with constant stirring. After 30 minutes, ice-bath was removed and the reaction was allowed to stir at room temperature for the next 4 h. Next, the solvent was evaporated and the residual was adsorbed on silica gel. The purification of crude material using flash chromatography (DCM:MeOH 95:5, v/v) provided 3.8 g (64.73%) of mannose monomer: C12H21NO7, ESI-MS [M+Na]+theor=m/z 314.1216, [M+Na]+found=m/z 314.1208; 1H NMR (400 MHz, D2O) δ 5.6 (s, 1H), 5.38 (s, 1H), 4.78 (s, 1H), 3.84 (s, C2, 1H), 3.77-3.34 (multiple signals from 7H: C3, C4, C5, C6, C6′, ethoxy group), 1.85 (s, 3H); 13C NMR (100 MHz, D2O) δ 172.06, 139.06, 121.00, 99.63, 72.78, 70.47, 69.99, 66.58, 65.73, 60.78, 39.04, 17.68.

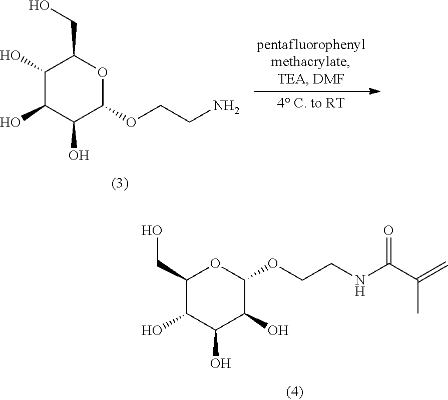 [SMILES: NCCO[C@H]1O[C@H](CO)[C@@H](O)[C@H](O)[C@@H]1O ; C=C(C)C(=O)NCCO[C@H]1O[C@H](CO)[C@@H](O)[C@H](O)[C@@H]1O]

### Compound 17: 2-(2-(2-(2-hydroxyethoxy)ethoxy)ethoxy)ethyl 4-methylbenzenesulfonate

Tetraethylene glycol (2.5 g, 12.9 mmol) and pyridine (1.0 g, 12.6 mmol) were added to 50 mL of DCM and stirred for 20 minutes at 0° C. To that solution, p-toluenesulfonyl chloride (2.37 g, 10 mmol) in 15 mL of DCM was added slowly. The reaction mixture was then stirred for 2 h at 0° C. followed by 4h at room temperature. After that time, the solvent was evaporated and crude product was purified via flash chromatography (ethyl acetate: hexane 6:4, v/v). The final yield was 1.95 g (72.22%): C15H2407S, ESI-MS [M+H]+theor=m/z 349.1321, [M+H]+found=m/z 349.1325; 1H NMR (400 MHz, CDCl3) δ 7.68 (d, 2H), 7.24 (d, 2H), 4.00-4.12 (m, 2H), 3.42-3.70 (m, 14H), 2.89 (t, 1H), 2.33 (s, 3H); 13C NMR (100 MHz, DMSO-d6) δ 144.68, 132.76, 129.68, 127.73, 72.34, 70.46, 70.41, 70.22, 70.10, 69.17, 68.44, 61.40, 21.41.

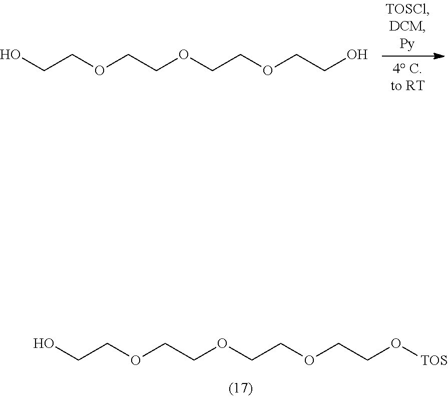

### Compound 18: 2-(2-(2-(2-azidoethoxy)ethoxy)ethoxy)ethan-1-ol

Sodium azide (1.5 g, 23.1 mmol) was added to a solution of 17 (1.5 g, 4.3 mmol) in N,N-dimethylformamide (75 mL) at room temperature. The reaction mixture was stirred overnight at 90° C. The reaction was then filtered and the solvent was removed in vacuo. The resulting viscous liquid was then purified by flash column chromatography (ethyl acetate: hexane 6:4, v/v) to yield a pure product (1.25 g, 83%): C8H17N304, ESI-MS [M+Na]+theor=m/z 242.1117, [M+Na]+found=m/z 242.1171; 1H NMR (400 MHz, DMSO-d6) δ 3.49-3.65 (m, 14H), 3.30 (t, 2H), 2.91 (t, 1H); 13C NMR (100 MHz, DMSO-d6) δ 72.43, 70.56, 70.52, 70.46, 70.21, 69.91, 61.51, 50.54.

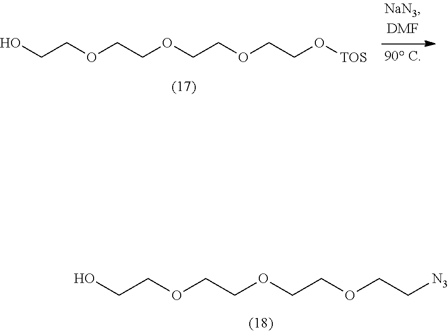

### Compound 19: 2-(2-(2-(2-azidoethoxy)ethoxy)ethoxy)ethyl 4-cyano-4(((dodecylthio) carbonothioyl) thio)pentanoate

4-Cyano-4-[(dodecylsulfanylthiocarbonyl)sulfanyl]pentanoic acid (500 mg, 1.24 mmol), compound 18 (226 mg, 1.03 mmol) and DCC (255 mg, 1.24 mmol) were dissolved in 4 mL of DCM. The solution was placed in an ice bath and let stir for 30 minutes. After that time, 4-dimethylaminopyridine (12.6 mg, 0.10 mmol) dissolved in 1 mL of DCM was added drop-wise. The solution was stirred at 0° C. for 2 h and then at room temperature for 1 hour. Once reaction was completed, DCM was evaporated in vacuum. The crude product was purified via column chromatography (DCM:ethyl acetate 97:3, v/v) to yield 300 mg (88.57%) of the pure product: C27H48N4O5S3, ESI-MS [M+H]+theor=m/z 604.2865 [M+H]+found=m/z 604.2862; 1H NMR (400 MHz, CDCl3) δ 4.26 (t, 2H), 3.76-3.65 (m, 12H), 3.39 (t, 2H), 3.33 (t, 2H), 2.66 (dd, 2H), 2.53 (m, 1H), 2.38 (m, 1H), 1.88 (s, 3H), 1.69 (dt, 2H), 1.38 (m, 2H), 1.26 (s, 16H), 0.88 (t, 3H); 13C NMR (100 MHz, CDCl3) δ 216.92, 171.46, 119.00, 70.72, 70.68, 70.61, 70.07, 68.96, 64.16, 50.69, 46.35, 37.06, 33.81, 31.91, 29.71, 29.62, 29.54, 29.42, 29.34, 29.07, 28.93, 27.68, 24.84, 22.69, 14.13.

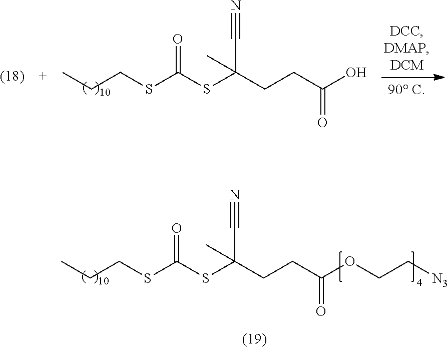

### Compound 21: p (Man)

The mannose monomer (4) (150.0 mg, 0.51 mmol) and (20) (130.0 mg, 1.03 mmol) were dissolved in 600 μl of anhydrous DMF and added to a schlenk tube. Then, RAFT agent (19) (18.5 mg, 0.03 mmol) in 100 μl of DMF and AIBN (1 mg, 0.006 mmol) in 10p of DMF were added to the schlenk tube. The tube was degassed via four freeze-pump-thaw cycles and then immersed in an oil bath preheated at 70° C. to initiate polymerization. The reaction was left stirring for 14 hours. After that time, the polymer was precipitated by transferring its viscous solution into 20 mL of cold acetone. The light-yellow suspension was placed in the freezer for 30 minutes. The precipitate was then centrifuged and re-suspended in fresh acetone. The processed was repeated 3 times. In the final step, the resultant glycopolymer (150 mg, 47.6%) was dried in a vacuum oven at reduced pressure and characterized by means of 1H NMR and GPC. The p(HPMA-TLR7) used in the biological studies had a number average molecular weight of 15,425 Da, as determined by size exclusion chromatography, using a dextran standard, a degree of polymerization of 82.1, and were composed of a 1:2.25 molar ratio of Mannose:HPMA, as determined by 1H NMR.

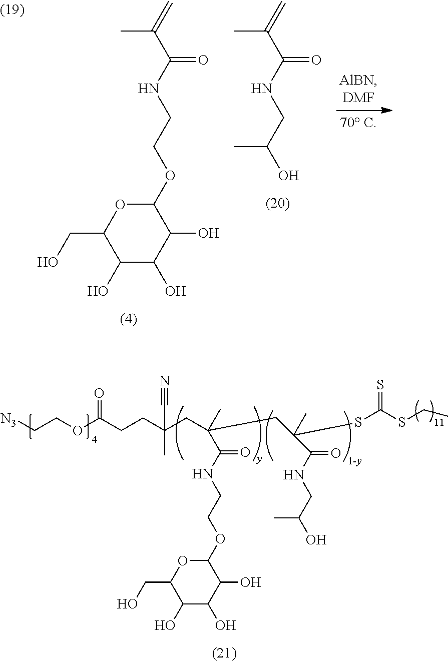

### Compound 25: Ethanol disulfanyl polyethylene glycol amine

A solution of thiol polyethylene glycol amine (JenKem Technology, USA) (1.0 g, 0.5 mmol) in DCM (5 ml) was added dropwise to a stirred solution of 2-(2-pyridinyldithio)ethanol (24) (467.5 mg, 2.5 mmol) in MeOH (3 ml). The solution was stirred at room temperature for 10 h then approximately half the solvent was removed via rotary evaporation. The remaining crude product was then decanted into ice cold hexanes (40 ml) and placed at −20° C. for 4 h. The precipitate and solvent mixture was centrifuged at 2000 g for 3 min. The solvent was then decanted and excess solvent was removed from the pelleted precipitate under reduced pressure. The crude product was then used in the next step without further purification (65% crude yield). The final structure was characterized by 1H NMR and reverse phase chromatography.

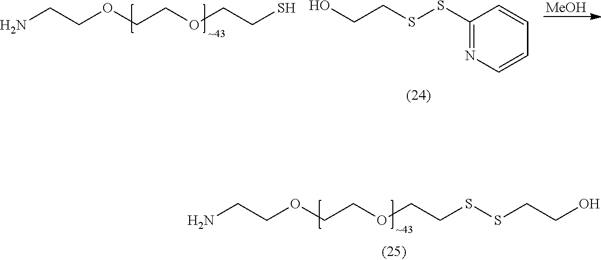

### Compound 26: Ethanol Disulfanyl Polyethylene Glycol (1R,8S,9s)-Bicyclo[6.1.0]non-4-yn-9-ylmethyl Carbamate

A solution of (1R,8S,9s)-Bicyclo[6.1.0]non-4-yn-9-ylmethyl N-succinimidyl carbonate (90 mg, 0.30 mmol) in DCM (0.5 ml) was added dropwise to an ice-cooled stirred solution of ethanol disulfanyl polyethylene glycol amine 25 (0.5 g, 0.24 mmol) and trimethylamine (48 mg, 0.48 mmol) in DCM (5 ml). After the addition of (1R,8S,9s)-Bicyclo[6.1.0]non-4-yn-9-ylmethyl N-succinimidyl carbonate, the reaction was allowed to come to room temperature and stirred for another 6 h. The reaction mixture was then poured into ice-cold hexanes (40 ml) and placed at −20° C. for 4 h. The precipitate and solvent mixture was centrifuged at 2000 g for 3 min. The solvent was then decanted and excess solvent was removed from the pelleted precipitate under reduced pressure. The crude product was then used in the next step without further purification (75% crude yield). The final structure was characterized by 1H NMR and reverse phase chromatography.

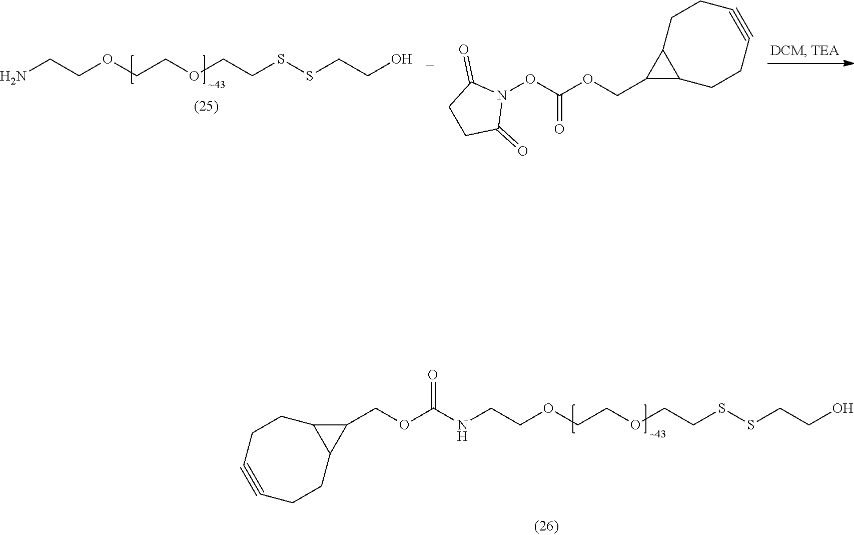

### Compound 27: N-Succinimidyl Carbamate Ethanol Disulfanyl Polyethylene Glycol (1R,8S,9s)-Bicyclo[6.1.0]non-4-yn-9-ylmethyl Carbamate (Self-Immolative Linker)

A solution of Ethanol disulfanyl polyethylene glycol (1R,8S,9s)-Bicyclo[6.1.0]non-4-yn-9-ylmethyl carbamate (300 mg, 0.13 mmol) in anhydrous acetonitrile (ACN)(1.5 ml) was added dropwise to a stirred solution of N,N′-Disuccinimidyl carbonate 26 (0.5 g, 0.24 mmol) and trimethylamine (48 mg, 0.48 mmol) in anhydrous ACN (5 ml). The reaction mixture was stirred overnight and was then poured into ice-cold hexanes (40 ml) and placed at −20° C. for 4 h. The precipitate and solvent mixture was centrifuged at 2000 g for 3 min. The solvent was then decanted and excess solvent was removed from the pelleted precipitate under reduced pressure. The crude product was purified via silica gel flash chromatography trough a thin pad of silica DCM:MeOH (85:15) (yield: 43%, 129 mg). The final structure was characterized by 1H NMR and reverse phase chromatography.

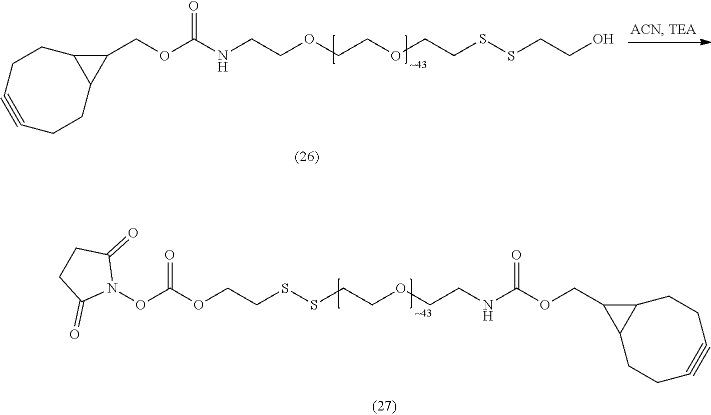

### Compound 28: General Procedure for OVA-, Asparaginase-Self-Immolative Linker Conjugates

EndoGrade® Ovalbumin (OVA) (Hyglos) (10 mg, 222.2 nmol), recombinant asparaginase (7.2 mg, 222.2 nmol) and self-immolative Linker (27) (5 mg) were added to an endotoxin free tube. Phosphate buffer at pH 7.7 (200 μL) was added to the tube and the tube was stirred at 1 h at room temperature. The reaction mixture was then filtered (0.22 μM) and the conjugates were purified via Zeba Spin Desalting Columns with a 30 kDa cutoff limit (Thermo Fisher). Chemical conjugation was verified via gel electrophoresis and high pressure size exclusion chromatography.

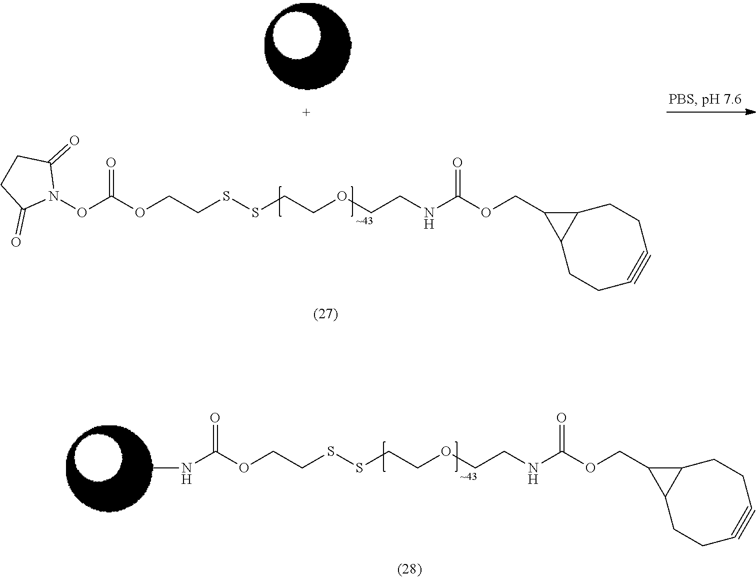

### Compound 29

Compound 28 in PBS, prepared as described above, was added to an endotoxin free tube and p(Man) (21) (30 mg) was added and the reaction was stirred for 30 min at room temperature. The reaction mixture was then filtered (0.22 μM) and the final product was purified via size exclusion chromatography. Chemical conjugation was verified via gel electrophoresis and high pressure size exclusion chromatography.

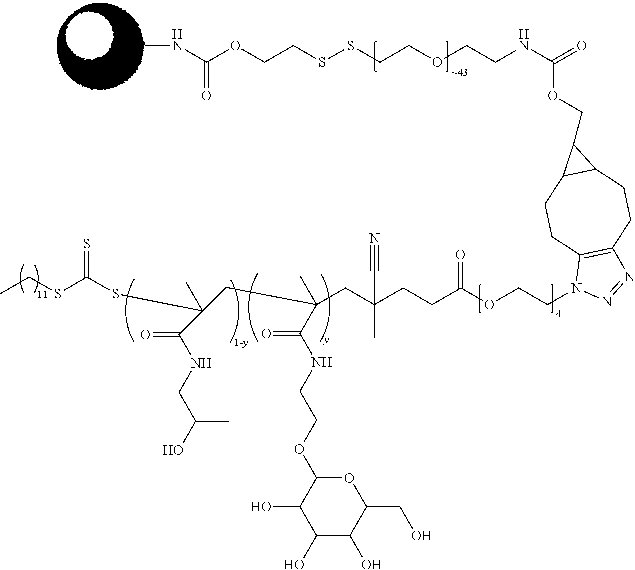

### Example 2

### OTI/OTII Challenge to Tolerance Model

BLK6 mice were treated with saline, or 10 μg of OVA (as a non-limiting example of an immunogenic antigen) in the form of free OVA or OVA conjugated to p(Man) (OVA-p(Man)) one day and 7 days after an adoptive transfer of 7.0×10⁵ OTI and OTII T cells. These mice were challenged with an intradermal injection of LPS and OVA 14 days after the initial OTI and OTII T cell transfer and then the immune response in the draining lymph nodes (dLNs) was assessed on day 19 and compared to mice that were treated with saline, but did not receive the challenge of LPS and OVA (No-challenge).

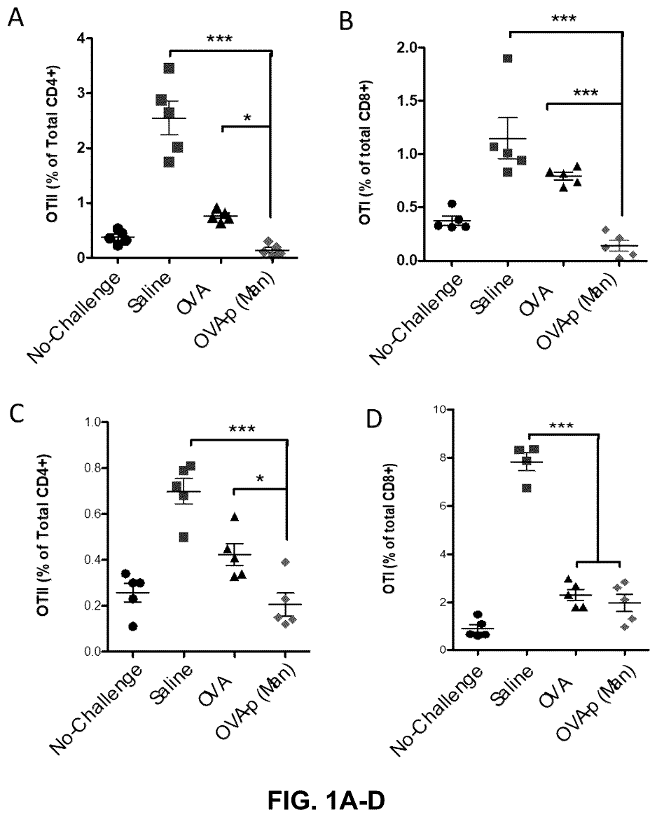

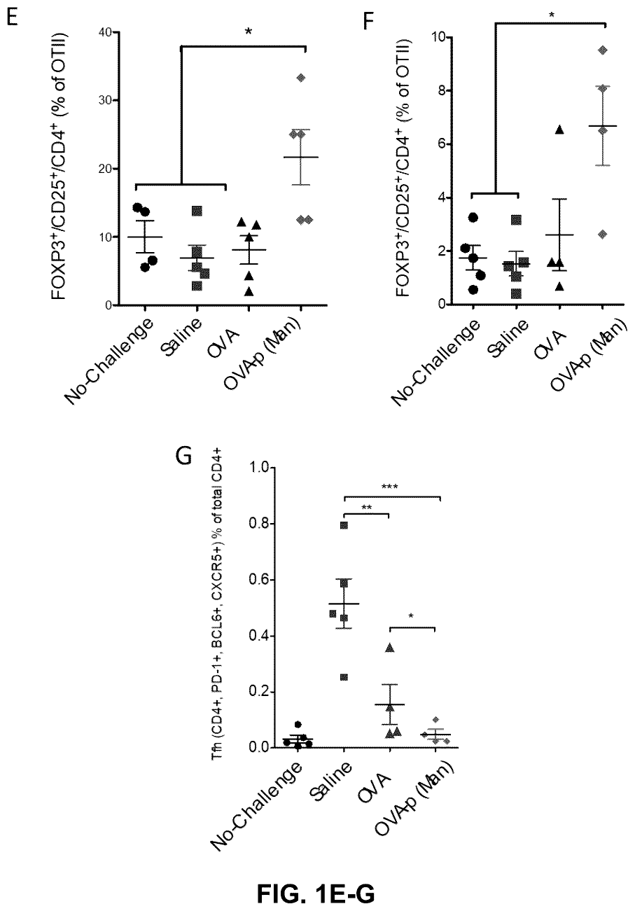

Profound tolerance was induced in the CD4+ T cell compartment, as shown in FIGS. 1-2. In terms of total cell frequencies, both dosing regimens of both OVA-p(Man) resulted in equivalent low levels of OTII cells after challenge, statistically lower than by treatment of OVA (*indicates p<0.05, ** indicates p<0.01), as shown in FIG. 1. When the cells that remained were analyzed by flow cytometry for the presence of the transcription factor FoxP3 and the receptor CD25, the numbers of FoxP3+CD25+ cells (markers of T regulatory cells) was statistically significantly elevated compared to treatment with OVA alone, as shown in FIG. 1. Additionally, the spleens of animals treated with OVA-p(Man) contained a lower percentage of T follicular helper cells (Tfh) as compared to other groups that were challenged with LPS and OVA. When the cells that remained were analyzed by flow cytometry for the expression of IFN-γ after exposure to OVA antigen, the frequency of CD4+ T cells expressing this inflammatory cytokine was decreased in the groups receiving OVA-p(Man), as shown in FIG. 2.

Profound tolerance was also induced in the CD8+ T cell compartment, as shown in FIG. 1. In terms of total cell frequencies, OVA-p(Man) resulted in equivalent low levels of OTI cells after challenge, statistically lower than by treatment of OVA (*indicates p<0.05, ** and indicates p<0.01), as shown in FIG. 1. When the cells that remained were analyzed by flow cytometry for the expression of IFN-γ after exposure to SIINFEKLE antigen, the frequency of CD8+ T cells expressing this inflammatory cytokine was decreased in the groups receiving OVA-p(Man), as shown in FIG. 2.

### Example 3

### Tolerance Induction to Intravenously Administered Asparaginase

Five BALB/c mice per group were injected with 2.5 μg of asparaginase (as a non-limiting example of an immunogenic antigen) formulated as free asparaginase (ASNase) or conjugated to p(Man) (ASNase-p(Man)) once a week for 3 weeks and then, at week 4, were switched to 15sig of ASNase i.v. once a week for 8 weeks. During the initial 3 weeks ASNase-p(Man) was administered via either i.v. or subcutaneous injection. Sera was taken from the mice and monitored weekly for the presence of αASNase.

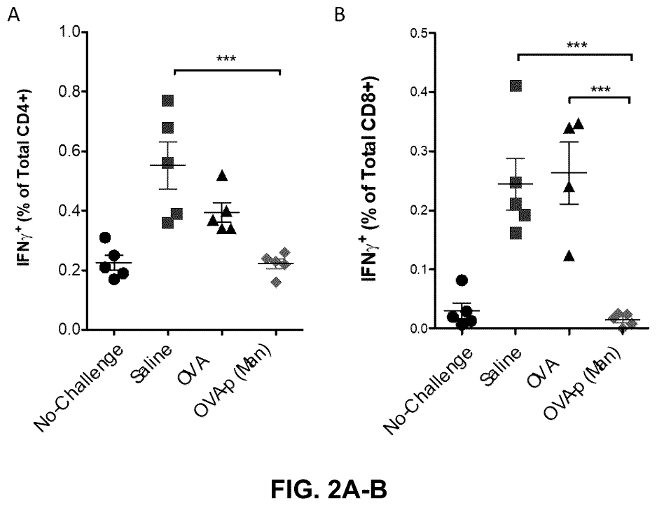

Upon intravenous injection of ASNase at week 4, animals treated with saline and subcutaneously administered ASNase-p(Man) experienced a rapid increase in serum αASNase IgG (FIG. 3), animals treated with i.v. administered ASNase-p(Man) did not incur an increase in serum αASNase IgG for the duration of the experiment. Furthermore, after 38 days of treatment, animals treated with ASNase-p(Man) via intravenous infusion, experienced significantly lower αASNase IgG subclass titers (FIG. 3).

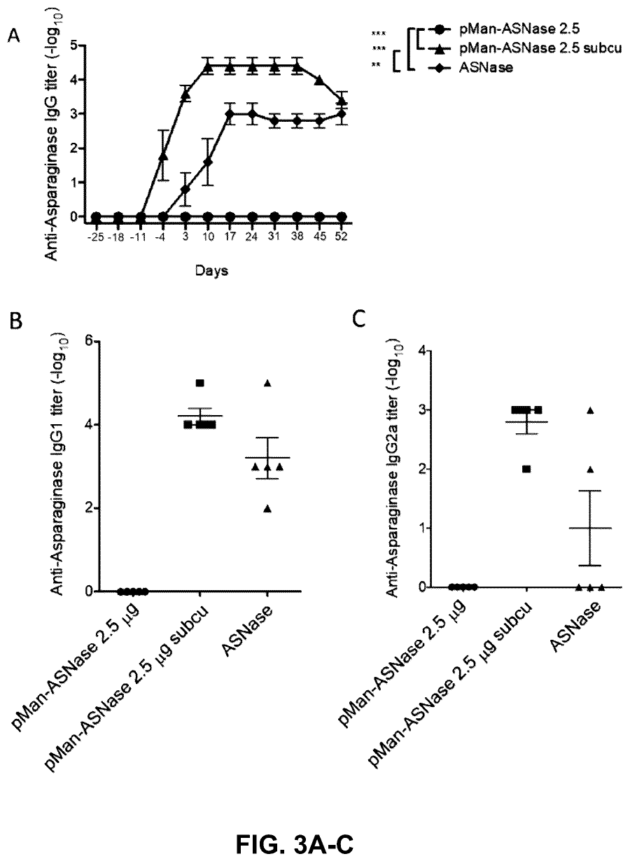

At week 22 (3 weeks of tolerization, 8 weeks of ASNase treatment, and 11 weeks after last dose of Asnase), animals treated with saline and ASNase-p(Man) via intravenous infusion were sacrificed and the spleens, livers, and bone marrow of these animals was collected and processed into single cell suspensions. The cells from these organs were stimulated in vitro with recombinant ASNase lacking activity for 3 days. After three days the cells were analyzed via flow cytometry for the existence of αASNase producing cells (plasma cells) and IL-10 producing B regulatory cells. The results show that the bone marrow of animals treated with ASNase-p(Man) had a fewer αASNase plasma cells than animals that were treated with ASNase (FIG. 4). On the contrary, the spleens of animals treated with ASNase-p(Man) had a greater percentage of IL-10 producing B regulatory cells.

### Example 4

### Effect on Anti-Asparaginase (Anti-ASNase) Humoral Immune Response

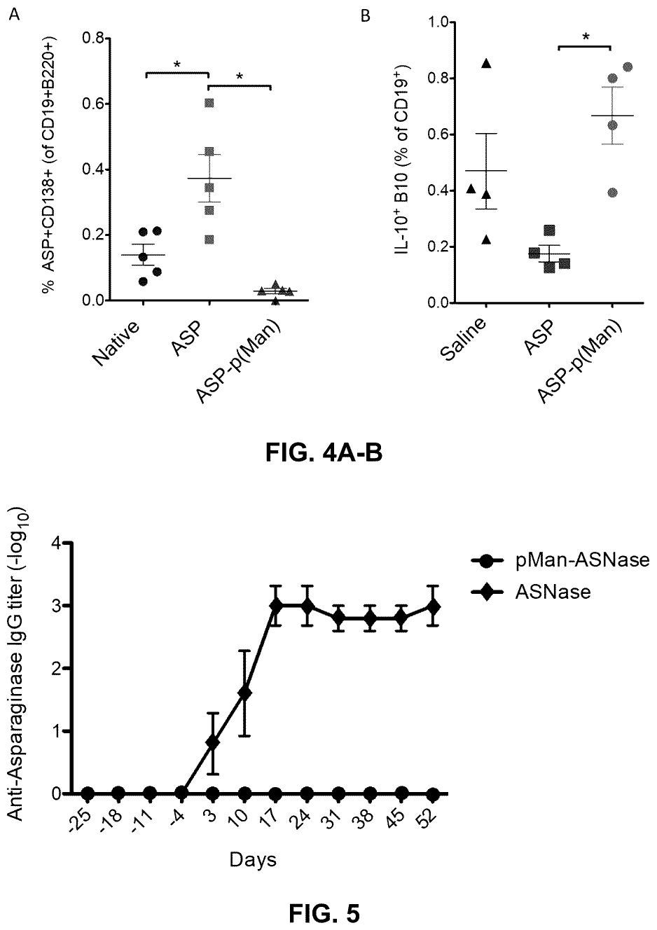

To assess the ability of p(Man)-protein conjugates to prevent an anti-asparaginase (anti-ASNase) humoral immune response and thus avoid the loss of efficacy that is the result of rapid antibody-mediated clearance of biological therapeutics form the serum, mice were treated on days −21, −14, and −7 with saline or 2.5 μg of ASNase conjugated to p(Man)(p(Man)-ASNase) via iv infusion (n=5 animals per group). After 7 days, the mice were then treated with weekly iv infusions of 15 μg of wt ASNase for 4 weeks. The mice were then treated with 15 μg of wt ASNase on day 49. On day 70, mice that had been treated with saline on days −21, −14, −7, 0, 14, 21, and 49 were treated with 15 μg of wt ASNase to assess the efficacy of ASNase in naive mice (FIG. 6). On day 70, the mice that had received saline or p(Man)-ASNase then treated with wt ASNase were also administered 15 μg of wt ASNase. The serum asparagine concentration of each animal was assessed on days 71, 73, and 76.

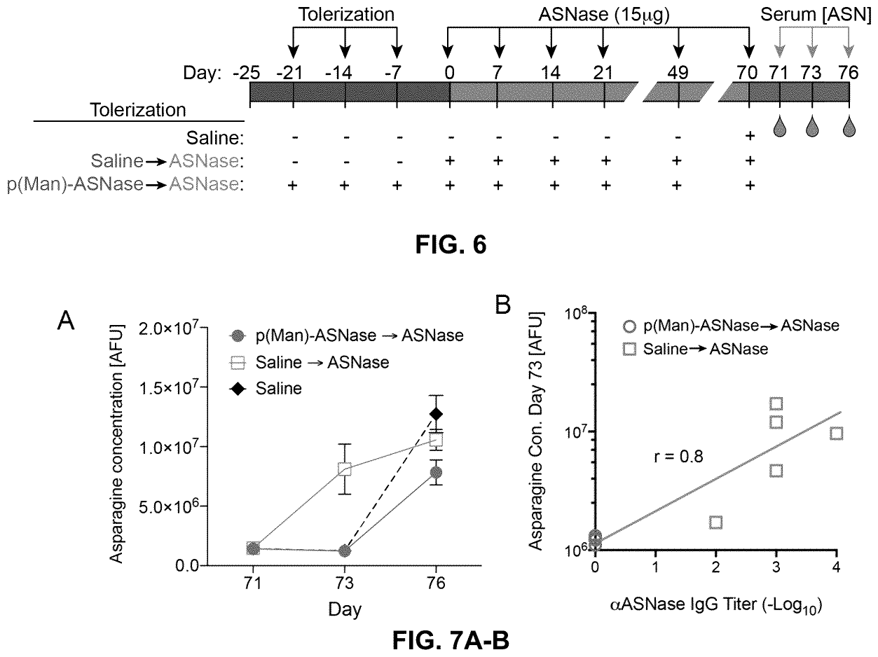

The results show that one day after being treated with ASNase, animals in each group have a similar serum concentration of asparagine. However, three days after being administered asparagine on day 7, animals treated with only saline or p(Man)-ASNase have a significantly lower serum asparagine concentration as compared to animals that had been treated with saline and then administered wt ASNase on days 0, 7, 14, 21, and 49 (FIG. 7A). In addition, animals treated with p(Man)-ASNase had significantly lower asparagine serum concentrations on day 76 than the animals in other groups. When serum asparagine concentration is plotted against the anti-ASNase titer for each animal in the study, a strong correlation (r=0.8) between serum asparagine concentration and anti-ASNase titer becomes evident (FIG. 7B). These results demonstrate that p(Man)-ASNase inhibits the loss of efficacy associated with an anti-ASNase immune response.

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A compound of Formula 1: 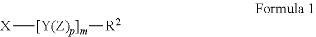
where:
X comprises an antigen, a tolerogenic portion thereof, or a mimetic thereof;
Y comprises a linker moiety;
Z comprises a moiety that specifically targets a mannose receptor;
p is an integer from 2 to 250;
m is an integer from 1 to 100;
R² is any of functional groups I-III: 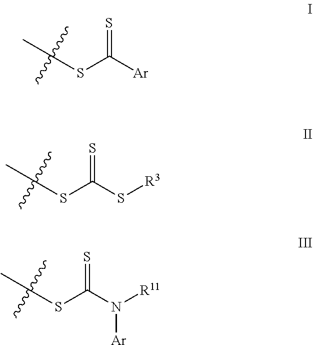
where Ar is a substituted or unsubstituted aromatic group,
R³ is any carbon-containing linear or heterocyclic moiety, and R¹¹ is hydrogen or an alkyl group.

2. The compound of claim 1, wherein the moiety that specifically targets a mannose receptor is selected from the group consisting of α-linked mannose, β-linked mannose, substituted mannose, mannose-6-phosphate, N-acetyl mannosamine, and mannan having β(1-4), α(1-6), α(1-2), and/or α(1-3) linkages.

3. The compound of claim 1, wherein Y is a linker resulting from reaction of at least one of a N-hydroxysuccinamidyl linker, maleimide linker, PEG linker, vinylsulfone linker, pyridyl di-thiol-poly(ethylene glycol) linker, pyridyl di-thiol linker, n-nitrophenyl carbonate linker, NHS-ester linker, and nitrophenoxy poly(ethylene glycol)ester linker.

4. The compound of claim 3, wherein Y is covalently bound to X.

5. The compound of claim 1, wherein —[Y(Z)ₚ]— is represented by one of Formula Ya to Yr: 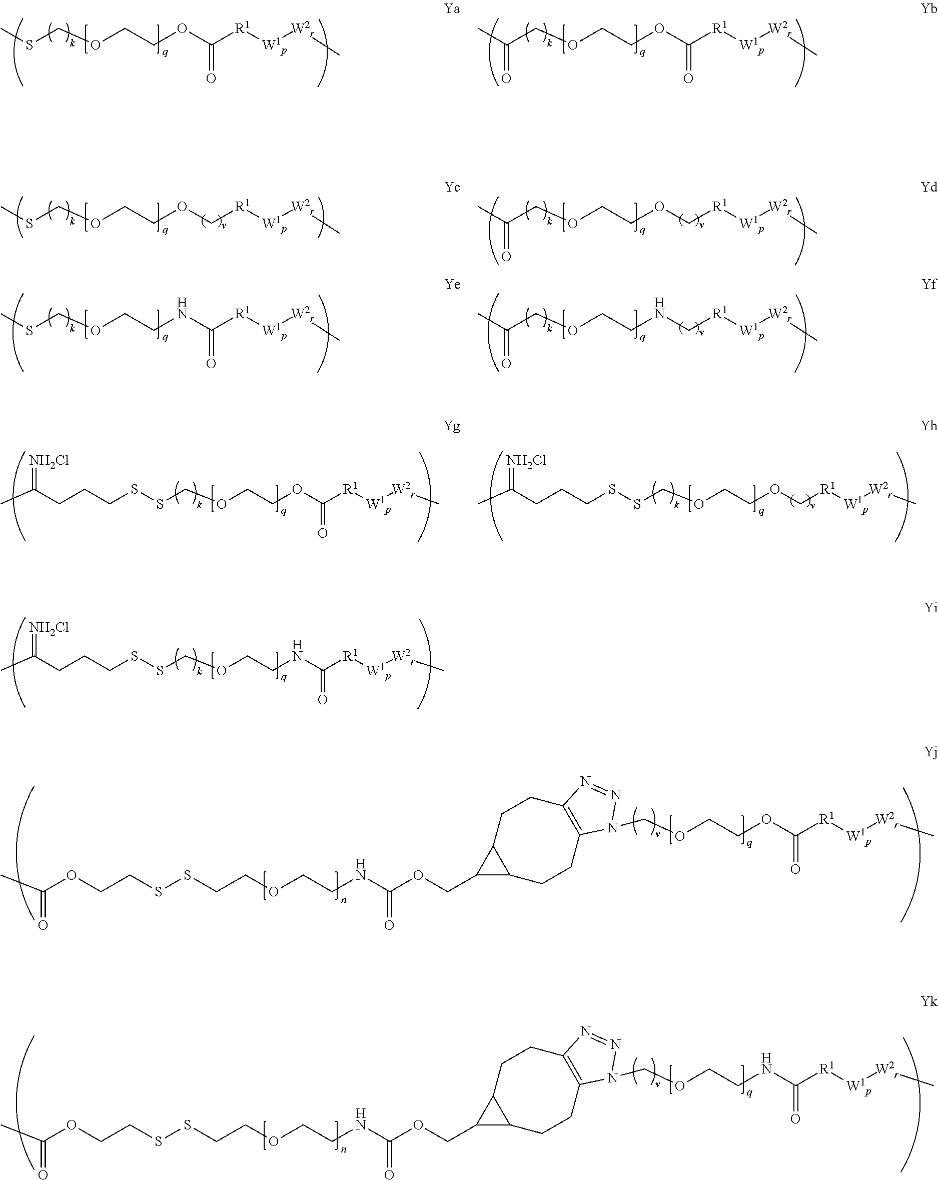 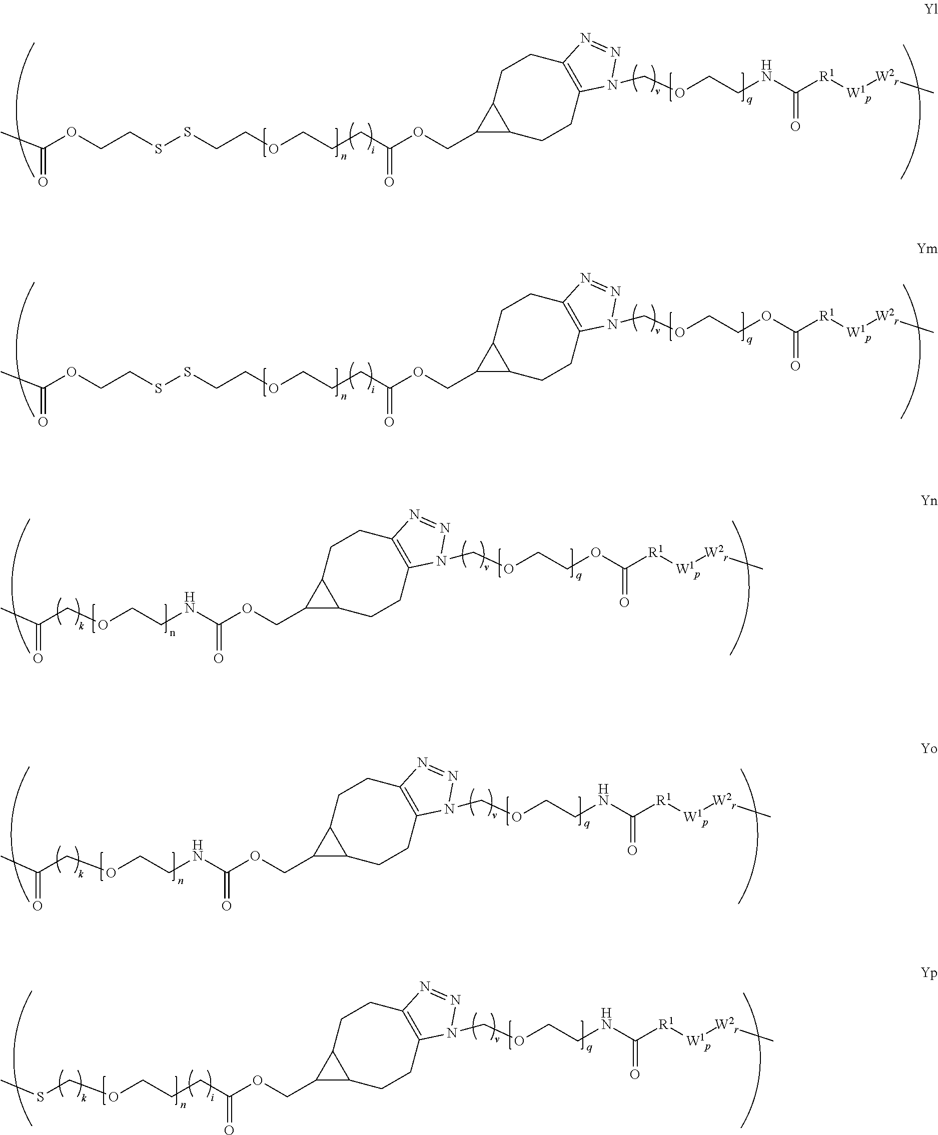 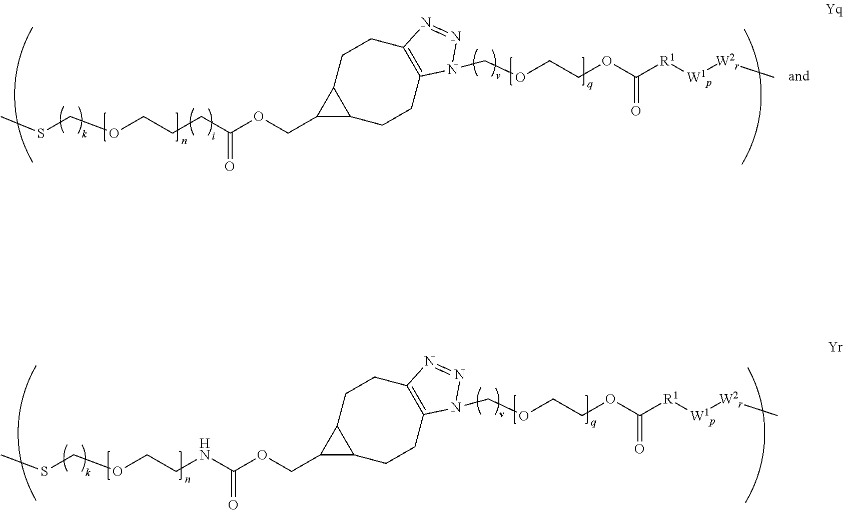
where
n is an integer from 1 to 100;
q is an integer from 1 to 44;
k is an integer from 1 to 12;
i is an integer from 0 to 20;
v is an integer from 1 to 4;
p is an integer from 2 to 250;
r is an integer from 0 to 250;
R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—;
W¹ and W² are as defined below: 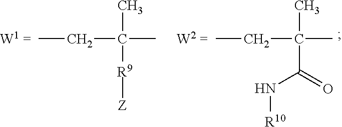
R⁹ is a direct bond, —(CH2)2-NH—C(O)— (an ethylacetamido group or “EtAcN”) or —(CH2)2-(O—CH2-CH2)t-NH—C(O)— (a pegylated ethylacetamido group or “Et-PEGt-AcN”)
t is an integer from 1 to 5;
Z is mannose or a mannose receptor-targeting moiety; and
R¹⁰ is an aliphatic group, an alcohol, an aliphatic amine-containing group, or an aliphatic alcohol.

6. The compound of claim 1, wherein Y is an antibody, antibody fragment, peptide or other ligand that binds to X.

7. The compound of claim 1, wherein X is an antigen against which a patient may develop or has developed an unwanted immune response.

8. The compound of claim 7, wherein the antigen is a foreign transplant antigen, an alloantigen, an autoimmune antigen, a food antigen, an animal antigen, a plant antigen, an environmental antigen, a therapeutic antigen, a synthetic self-antigen, or a tolerogenic portion thereof.

9. The compound of claim 8, wherein X is an asparaginase antigen or an ovalbumin antigen.

10. The compound of claim 1, wherein the mannose receptor is mannose-6-phosphate receptor.

11. The compound of claim 1, wherein Y and X are connected through a bond configured to cleave when the compound reaches a target area.

12. The compound of claim 1, wherein Ar is selected from: 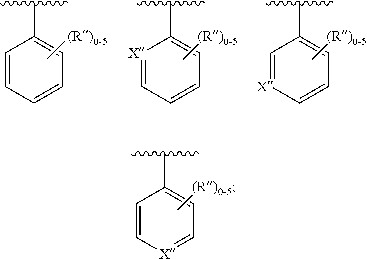
where each instance of R″, where present, is independently selected from an optionally substituted C₁₋₆-alkyl, optionally substituted C₁₋₆ alkoxy, optionally substituted amino, OH, or halogen and wherein, X″ is a heteroatom.

13. The compound of claim 12 wherein X″ is N.

14. The compound of claim 1, wherein R¹¹ is C₁₋₆-alkyl.

15. The compound of claim 1, wherein R¹¹ is —CH₃.

16. The compound of claim 1, wherein R³ is C₁₋₆-alkyl.

17. A composition comprising the compound of claim 1.

18. (canceled)

19. A method of inducing immunological tolerance to an antigen target comprising administering to a subject a composition Formula 1: 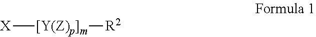
where:
X comprises an antigen, a tolerogenic portion thereof, or a mimetic thereof;
Y comprises a linker moiety;
Z comprises a moiety that specifically targets a mannose receptor;
p is an integer from 2 to 250;
m is an integer from 1 to 100;
R² is any of functional groups I-III: 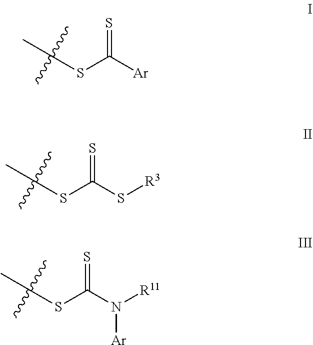
where Ar is a substituted or unsubstituted aromatic group, R³ is any carbon-containing linear or heterocyclic moiety, and R¹¹ is hydrogen or an alkyl group.

20. The method of claim 19, wherein the moiety that specifically targets a mannose receptor is selected from the group consisting of α-linked mannose, β-linked mannose, substituted mannose, mannose-6-phosphate, N-acetyl mannosamine, and mannan having β(1-4), α(1-6), α(1-2), and/or α(1-3) linkages.

21-48. (canceled)

49. A method for treating or preventing multiple sclerosis comprising, administering to a subject having, or predicted to have, multiple sclerosis, a composition comprising a compound of Formula 1: 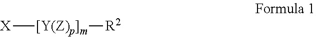
where:
X comprises an autoimmune antigen, a tolerogenic portion thereof, or a mimetic thereof;
Y comprises a linker moiety;
Z comprises a moiety that specifically targets a mannose receptor;
p is an integer from 2 to 250;
m is an integer from 1 to 100;
R² is any of functional groups I-III: 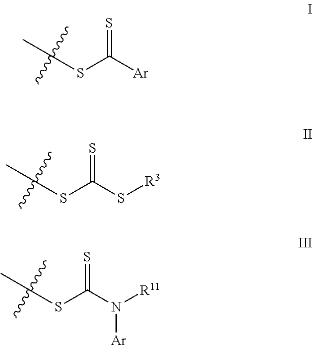
where Ar is a substituted or unsubstituted aromatic group,
R³ is any carbon-containing linear or heterocyclic moiety, and R¹¹ is hydrogen or an alkyl group.

50-60. (canceled)
